(19) United States
(12) Patent Application Publication
(10) Pub. No.: US 2024/0271163 A1
(43) Pub. Date: Aug. 15, 2024

(54) ENGINEERED GUIDE RNA SCAFFOLDS AND METHODS THEROF FOR ENHANCED GENOME EDITING

(71) Applicants: Versitech Limited, Hong Kong (CN); Centre for Oncology and Immunology Limited, Hong Kong (CN)

(72) Inventors: Siu Lun Wong, Hong Kong (CN); Hoi Yee Chu, Hong Kong (CN); Peng Zhou, Hong Kong (CN); Hoi Chun Fong, Hong Kong (CN)

(21) Appl. No.: 18/441,806

(22) Filed: Feb. 14, 2024

Related U.S. Application Data
(60) Provisional application No. 63/484,902, filed on Feb. 14, 2023.

(51) Int. Cl. C12N 15/90; C12N 9/22; C12N 15/11

(52) U.S. Cl. CPC C12N 15/907; C12N 9/22; C12N 15/11; C12N 15/111; C12N 2310/20; C12N 2310/531; C12N 2800/80

(57) ABSTRACT

Engineered guide RNAs having enhanced stability of interaction with Cas enzymes are disclosed. The variant sgRNAs include engineered nucleic acids in or around the stem-loop 2 region which enhance interaction with the Cas9 enzyme and impart enhanced specificity and on-target editing activity. Compositions and methods of engineered guide RNAs are provided for enhanced genomic engineering with increased on-off target specificity and on-target editing efficacy.

### CROSS-REFERENCE TO RELATED APPLICATIONS

This application claims benefit of and priority to U.S. Patent Application No. 63/484,902, filed on Feb. 14, 2023, the contents of which is hereby incorporated by reference herein in its entirety.

### REFERENCE TO SEQUENCE LISTING

The Sequence Listing XML submitted as a file named “UHK_01282_PCT_ST26.xml”, and having a size of 323,020 bytes is hereby incorporated by reference pursuant to 37 C.F.R. § 1.834(c)(1).

### FIELD OF THE INVENTION

The invention is generally in the field of genetic engineering and specifically in the area of CRISPR/Cas based genome editing using guide RNAs designed for enhanced stability and specificity.

### BACKGROUND OF THE INVENTION

CRISPR-Cas9 systems hold great promise for applying genome editing to biomedicine. CRISPR-Cas9 is a programmable gene-editing system that can be used to knock out genes and correct genetic mutations in human cells (Anzalone, et al., Nat Biotechnol. 2020, 38, (7), 824-844). This system utilizes a single guide RNA (sgRNA) that directs the Cas9 protein to the target genomic site for editing. Existing CRISPR/Cas9 toolkits exhibit varying efficiencies across loci, limiting their applicability for therapeutic genome editing. Optimization of such systems is in great need.

Applying genome editing technologies for applications in humans requires tools that are robust, reliable and specific, and a great deal of work has focused on enhancing the specificity of CRISPR/Cas9. Two main approaches have been taken to optimize CRISPR/Cas9 system activity: 1) by modification of the Cas9 protein and 2) by optimization of the sgRNA. Approaches involving Cas9 protein engineering have primarily focused on improving its specificity and targeting scope via directed evolution and targeted mutagenesis (Kleinstiver, et al., Nature 2016, 529, (7587), 490-5; Slaymaker, et al., Science 2016, 351, (6268), 84-8; Hu, et al., Nature 2018, 556, (7699), 57-63; Nishimasu, et al., Science 2018, 361, (6408), 1259-1262; Kleinstiver, et al., Nature 2015, 523, (7561), 481-5; Casini, et al., Nat Biotechnol 2018; Chen, et al., Nature 2017, 550, (7676), 407-410; Choi, et al., Nat Methods 2019, 16, (8), 722-730; Lee, et al., Nat Commun 2018, 9, (1), 3048; and Vakulskas, et al., Nat Med 2018, 24, (8), 1216-1224).

The other approach focuses on optimizing the sgRNAs used. The protospacer sequence of sgRNA is responsible for target site recognition, whereas its scaffold sequence binds to Cas9, which results in the conformational change of Cas9 for its activation. Many studies have been done on elucidating the determinants in the protospacer sequence for sgRNAs to exhibit high on-target and low off-target activities (Hanna, et al., Nat Biotechnol 2020, 38, (7), 813-823). However, specific loci, including therapeutically relevant ones, may have limited choices of protospacer sequences for targeting, and many protospacer sequences result in only a moderate or even low percentage of editing.

The scaffold sequence of sgRNA can be engineered to alter the overall editing activity by increasing its stability and assembly with the Cas9 protein. The “E+F” scaffold variant was engineered with a 5-nucleotide-extended tetraloop that could strengthen the scaffold's interaction with SpCas9 and an A-U base-pair flip in the lower stem that removes a putative polymerase-III terminator sequence (Chen, et al., Cell 2013, 155, (7), 1479-91). The E+F scaffold sequence was further mutated with different substitutions, and specific regions were identified to be more tolerant of mutations without compromising the sgRNA's activity (Jost, et al., Nat Biotechnol 2020, 38, (3), 355-364). Six scaffold variants, three of them containing additional U61C+A66G mutations besides those in the E+F scaffold, were reported to generate more edits. Apart from these efforts, there has been limited success in enhancing SpCas9's activity. Existing engineered guide RNA scaffolds that increase on-target editing of the widely used Streptococcus pyogenes Cas9 (SpCas9) nuclease greatly compromise its on-to-off targeting specificity. No guide RNA scaffold variant with both enhanced efficiency and high genome-wide accuracy has been described for SpCas9. No SpCas9 variant reported to date has exhibited enhanced activity. Also, whether these engineered scaffolds increase off-target edits, which is an important concern for applications of genome editing, has not been evaluated.

Therefore, it is an object of the invention to provide enhanced reagents and methods for CRISPR-Cas9 genomic engineering with enhanced on-site activity and greater specificity than existing reagents.

It is also an object of the invention to provide compositions and methods for genome editing with enhanced on-site activity and minimal off-targeting.

It is a further object of the invention to provide CRISPR-Cas9 editors that generate more edits to attain functional outcomes at loci associated with modest editing using wild type editors.

### SUMMARY OF INVENTION

Variant guide RNA scaffolds that impart enhanced editing activity and high genome-wide targeting specificity in human cells have been developed. The engineered variant guide RNA scaffolds implement activity-enhancing mutations that enhance their editing activities as compared with wild-type guide RNA scaffolds and pre-existing variants.

Variant single guide RNA (sgRNA) including substitution and/or addition of one or more nucleic acid residues that strengthens the interaction of the sgRNA with a Cas enzyme are provided. Typically, the strengthened interaction imparts increased on-target editing and/or increased on-off target specificity relative to a wild type sgRNA that lacks the substitution and/or addition of one or more nucleic acid residues. In some forms, the substitution and/or addition of one or more nucleic acid residues that strengthens the interaction of the sgRNA with a Cas enzyme includes substitution and/or addition of one or more nucleic acid residues within the stem-loop 2 region of the sgRNA. Typically, the Cas enzyme is a Cas9 enzyme, such as the Cas9 enzyme derived from Streptococcus pyogenes (SpCas9). In some forms, the substitution and/or addition of one or more nucleic acid residues strengthens the sgRNAs interaction with residue His721 and/or the PI domain of SpCas9.

In some forms, the variant sgRNA includes a framework region of a wild-type sgRNA having the nucleic acid sequence:

(SEQ ID NO: 354)
GUUUUAGAGCUAGAAAUAGCAAGUUAAAAUAAGGCUAGUCCGUUAUCA-
X-GGCACCGAGUCGGUGCU,

whereby “—X—” represents a hairpin region of stem-loop 2 including between 12 and 24 nucleic acid residues, inclusive. In some forms, the stem-loop 2 region includes the nucleic acid sequence of any one of SEQ ID NOS: 1-312. In particular forms, the stem-loop 2 region includes the nucleic acid sequence GCGGGGUGCCGC (SEQ ID NO:48), or a nucleic acid sequence having at least 75%, up to 99% identity to SEQ ID NO:48. In some forms, the variant sgRNA includes a hairpin region of stem-loop 2 having a nucleic acid sequence having at least 75% sequence identity to GCGGGGUGCCGC (SEQ ID NO:48). For example, in some forms, the variant sgRNA includes a hairpin region of stem-loop 2 having a nucleic acid sequence at least 80%, at least 85%, at least 90%, or at least 95% identical to GCGGGGUGCCGC (SEQ ID NO:48). In other forms, the stem-loop 2 region includes the nucleic acid sequence GGGCCGGGGUGCCGGCCC (SEQ ID NO:240), or a nucleic acid sequence having at least 75%, up to 99% identity to SEQ ID NO:240. In some forms, the variant sgRNA includes a hairpin region of stem-loop 2 having a nucleic acid sequence having at least 75% sequence identity to GGGCCGGGGUGCCGGCCC (SEQ ID NO:240). For example, in some forms, the variant sgRNA includes a hairpin region of stem-loop 2 having a nucleic acid sequence at least 80%, at least 85%, at least 90%, or at least 95% identical to GGGCCGGGGUGCCGGCCC (SEQ ID NO:240). In one form, a variant sgRNAs that imparts increased on-target editing and/or increased on-off target specificity relative to a wild type sgRNA includes the nucleic acid sequence:

(SEQ ID NO: 352)
GUUUUAGAGCUAGAAAUAGCAAGUUAAAAUAAGGCUAGUCCGUUAUCAG
CGGGGTGCCGCGGCACCGAGUCGGUGCU.

In some forms, the variant sgRNA includes a nucleic acid sequence having at least 75% sequence identity to SEQ ID NO:352. For example, in some forms, the variant sgRNA includes a nucleic acid sequence at least 80%, at least 85%, at least 90%, or at least 95% identical to SEQ ID NO:352. In another form, a variant sgRNAs that imparts increased on-target editing and/or increased on-off target specificity relative to a wild type sgRNA includes the nucleic acid sequence:

(SEQ ID NO: 353)
GUUUUAGAGCUAGAAAUAGCAAGUUAAAAUAAGGCUAGUCCGUUAUCAG
GGCCGGGGUGCCGGCCCGGCACCGAGUCGGUGCU.

In some forms, the variant sgRNA includes a nucleic acid sequence having at least 75% sequence identity to SEQ ID NO:353. For example, in some forms, the variant sgRNA includes a nucleic acid sequence at least 80%, at least 85%, at least 90%, or at least 95% identical to SEQ ID NO:353.

Ribonucleoprotein complexes including the variant sgRNAs are also described. Typically, the ribonucleoprotein complexes include: (a) a Cas9 enzyme; and (b) a variant sgRNA, whereby the variant sgRNA includes a stem-loop 2 region including the nucleic acid sequence of any one of SEQ ID NOs:1-312, and whereby the ribonucleoprotein complex has increased on-target editing and/or increased on-off target specificity relative to the corresponding complex between a Cas9 enzyme and wild type sgRNA. In some forms, the Cas9 enzyme is derived from Streptococcus pyogenes (SpCas9). Generally, the variant sgRNA includes a framework having the nucleic acid sequence:

(SEQ ID NO: 355)
GUUUUAGAGCUAGAAAUAGCAAGUUAAAAUAAGGCUAGUCCGUUAUCA-
X-GGCACCGAGUCGGUGCU,

whereby “—X—” represents the stem-loop 2 region including the nucleic acid sequence of any one of SEQ ID NOs: 1-312. In some forms, the ribonucleoprotein complex includes a variant sgRNA having the nucleic acid sequence:

(SEQ ID NO: 352)
GUUUUAGAGCUAGAAAUAGCAAGUUAAAAUAAGGCUAGUCCGUUAUCAGC
CACCGAGUCGGUGCU.

In some forms, the variant sgRNA includes a nucleic acid sequence having at least 75% sequence identity to SEQ ID NO:352. For example, in some forms, the variant sgRNA includes a nucleic acid sequence at least 80%, at least 85%, at least 90%, or at least 95% identical to SEQ ID NO:352. In some forms, the ribonucleoprotein complex includes a variant sgRNA having the nucleic acid sequence:

(SEQ ID NO: 353)
GUUUUAGAGCUAGAAAUAGCAAGUUAAAAUAAGGCUAGUCCGUUAUCAGG
GCCGGGGUGCCGGCCCGGCACCGAGUCGGUGCU.

In some forms, the variant sgRNA includes a nucleic acid sequence having at least 75% sequence identity to SEQ ID NO:353. For example, in some forms, the variant sgRNA includes a nucleic acid sequence at least 80%, at least 85%, at least 90%, or at least 95% identical to SEQ ID NO:353. Vectors encoding of expressing the variant sgRNA and/or the ribonucleoprotein complex thereof, and cells including these compositions are also provided.

Methods for CRISPR-based editing of one or more target genes in a cell are also provided. Generally, the methods include administering into and/or expressing within the cell the variant sgRNA and/or the ribonucleoprotein complex thereof, wherein the variant sgRNA is configured to target the one or more target genes. The administering can be in vitro or in vivo.

Kits including the variant sgRNAs are also disclosed. In some forms, the kits include instructions for performing a method of CRISPR-based editing of one or more target genes, and/or a Cas9 enzyme, or vector encoding or expressing the Cas9 enzyme.

### BRIEF DESCRIPTION OF DRAWINGS

FIGS. 1A-1B show a sequence alignment showing the Wild type (Wt) sgRNA scaffold (SEQ ID NO:345), as well as variants E+F (SEQ ID NO:346), E+F U61C/A66G (i.e., cr772) (SEQ ID NO:347), U61C/A66G (SEQ ID NO:348), E+F G62A/A64G (SEQ ID NO:349), G62A/A64G (SEQ ID NO:350), and 5E (SEQ ID NO:351) (FIG. 1A). The relative orientations of the sequences corresponding to structural regions tetraloop, nexus, stem-loop 2 and stem-loop 3 are indicated. FIG. 1B is a schematic representation of the structure of the wild-type sgRNA, indicating the spacer sequence, and other structural components (spacer, nexus, stem-loop 2 and stem-loop 3).

FIGS. 2A-2C are graphs depicting the comparative analysis of previously described and stem-loop 2-modified sgRNA scaffold variants. FIG. 2A is a histogram of On-target activity of sgRNA scaffold variants, showing RFP disruption rate (%) for each of wild type, E+F, E+F U61C/A66G (i.e., cr772), U61C/A66G, E+F G62A/A64G, G62A/A64G, and 5E. Values and error bars represent mean±S.D. (n=3). Statistical significance is analyzed by Tukey's HSD test against wild type (*P<0.05 and **** P<0.0001). FIGS. 2B-2C are graphs of On-target activity of sgRNA scaffold variants on endogenous loci analyzed by T7 endonuclease assay, showing normalized editing efficiency for each of cr772, E+F G62A/A64G and 5E, respectively (FIG. 2B), and in triplicate for each of the five loci, with the editing activity of the sgRNA scaffold variants was normalized against wild type, and their mean is indicated by a red line (n=5, one-sample t-test), respectively)(FIG. 2C). * indicates P<0.05; n.s. indicates not significant. On-target activity of sgRNA scaffold variants analyzed by RFP disruption assay. Values and error bars represent mean±S.D. (n=3). Statistical significance is analyzed by Tukey's HSD test against wild type (*P<0.05 and **** P<0.0001).

FIGS. 3A-3C are sequence alignments showing the On-to-off targeting activity of sgRNA scaffold variants on endogenous loci together with corresponding Read counts detected by GUIDE-seq for each of FANCFsg site 6 (FIG. 3A), EMX1sg site 3 (FIG. 3B) and PD-1 (FIG. 3C), respectively.

FIGS. 4A-4D are diagrams showing the molecular interface between SpCas9 and variant sgRNAs, showing: 5E strengthened existing interactions with H721 of SpCas9 and created new interactions with two regions (E1175-N1177 and K1192-D1193) of the PI domain in SpCas9. Wild-type sgRNA is overlaid for comparison (FIG. 4A); H721 is in closer proximity with the 5E) sgRNA backbone at the 5th nucleotide at the 3′ extension (A ext5) compared to the wild-type sgRNA closest nucleotide A65. Both sgRNAs maintain the potential backbone interaction at U55 with H721 (FIG. 4B); the stem-loop 2 extension of 5E creates new interactions with E1175, K1176, and N1177 with the 3^{rd} nucleotide at 3′ extension (G ext3), 1st nucleotide at 5′ extension (G ext1), G62 via sgRNA backbone and A64 via nucleotide base (FIG. 4C); and the stem-loop 2 extension of 5E creates new interactions with K1192 and D1193 with the 1st nucleotide at 3′ extension (C ext1) and A65 via the sgRNA backbone (FIG. 4D), respectively.

FIGS. 5A-5G depict activity profiling of stem-loop 2-engineered sgRNA scaffold variants identifies SV48 and SV240 that increase the editing efficiency of SpCas9 editors. FIG. 5A shows design of the sgRNA scaffold variant library: Focusing on stem-loop 2, combinations of beneficial mutations including 1) lengthening of 1-6 bp of the stem region, 2) base-pair mutations at 58-69, 60-67, and 61-66 bp, and 3) tetraloops that maximize sgRNA-protein interactions, were introduced. The relative orientations of the sequences corresponding to structural regions tetraloop, nexus, stem-loop 2 and stem-loop 3 are indicated on a diagram depicting the sgRNA. FIG. 5B is a schematic depicting the screening work-flow; library of 312 sgRNA scaffold variants was delivered into human cells expressing SpCas9 or base editor; genomic DNA was collected, and the region containing the sgRNA scaffold variant and its targeted reporter loci was subjected to deep sequencing. FIG. 5C is a dot plot graph of pooled screening results of sgRNA scaffold variants, showing base editing efficiency of sgRNA scaffold variants over Cas9 editing efficiency. Base editing efficiency when sgRNA scaffold variants were used with a SpCas9 nuclease or a base editor was computed from alleles identified by CRISPresso2. The top 5%-most active variants identified in SpCas9 nuclease-based screen are labelled. FIG. 5D is a diagram showing the sequence of sgRNA scaffold for each of wild type (WT), and variants SV48 and SV240, respectively. The stem-loop 2 region of WT (AACUUGAAAAAGUG; SEQ ID NO: 362); SV48 (AGCGGGGUGCCGCG; SEQ ID NO:363) and SV240 (AGGGCCGGGGUGCCGGCCCG; SEQ ID NO: 364) are shown. FIGS. 5E-5F are histograms showing cytosine base editing activity (FIG. 5E) and SpCas9 nuclease editing activity (FIG. 5F), respectively of sgRNA scaffold variants on endogenous target analyzed by deep sequencing. Values and error bars reflect mean±S.D. (n=3). Statistical significance was analyzed by Tukey's HSD test against wild type (WT) (*P<0.05, ** P<0.01, *** P<0.001 and **** P<0.0001). FIG. 5G is a sequence alignment showing the On-to-off targeting activity of sgRNA scaffold variants on endogenous loci together with corresponding Read counts detected by GUIDE-seq for each of CXCR4sg site 6, EMX1sg site 2 and HBG-sg4, respectively.

FIGS. 6A-6F are diagrams depicting the molecular interface between SpCas9 and variant sgRNAs, showing that: H721 is the solo amino acid at SpCas9 interacting with the stem-loop 2 of wild-type sgRNA (tan) and SV48 (sky blue) (FIG. 6A); SV48 containing a GGUG tetraloop and other substitutions in the stem-loop 2 regions has led to a slightly different loop conformation. The backbone of G65 and C66 is closer to H721 (3 Å) and forms two points of contacts for stronger interactions (FIG. 6B); A64 and A65 at the tetraloop of stem-loop 2 of the wild-type sgRNA are likely to interact with H721 due to close contacts (4-5 Å) (FIG. 6C); SV240 has strengthened existing interactions with H721 of SpCas9 and created new interactions with K1176 of the PI domain in SpCas9—wild-type sgRNA is overlaid for comparison. (FIG. 6D); H721 makes contacts with the backbone of the 3rd nucleotide at the 3′ extension (G ext3) of SV240 as it is 3-4 Å away from the RNA backbone (FIG. 6E); and K1176 is within 4 Å away from the backbone of G63 and U64 of the tetraloop of stem-loop 2 and makes contacts with the RNA backbone (FIG. 6F), respectively.

FIG. 7 is a graph showing RFP disruption rate (%) for each of wild type (WT), E+F U61C/A66G (i.e., cr772), and 5E, respectively. The off-target activity of sgRNA scaffold variants was analyzed by RFP disruption assay. The sgRNA spacer sequence and its target site (i.e., RFPsg5-OFF5-2) used contains a 1-bp mismatch (see Methods). Values and error bars represent mean±S.D. (n=3). Statistical significance is analyzed by Tukey's HSD test against wild type (*P<0.05).

FIG. 8 is a graph of Mean relative to wild type (WT) sgRNA expression (SD represented by grey error bar) of sgRNA scaffold variants known in the art were plotted against the described panel of sgRNA variants, ordered by increasing relative expression level. The WT expression is plotted. Variants with higher than WT expression (depicted in the box) are summarized in the schematic diagram about their beneficial mutations where positions enriched with beneficial mutations are shaded, and the bases of beneficial mutations are in boldface and unshaded.

FIGS. 9A-9C are diagrams of molecular models showing the effects of beneficial mutations by structural modelling using PDB 600Y as a template. FIG. 9A depicts the structural changes of swapping the wild-type “GAAA” tetraloop to “GAGA” tetraloop, resulting in changing the conformation from U-turn to Z-turn, leading to A63 exposing and potentially facing H721 for increased interaction. FIG. 9B depicts swapping the WT “GAAA” tetraloop to “GGUG”, which led to the “flipping” of G63, G64, and G65, facilitating base-residue interaction between 65G and H721. FIG. 9C depicts lengthening stem regions of stem-loop 2, (i.e., 3 bp extension), which brings the tetraloop closer to H721 and E722, thus promoting protein-sgRNA interactions.

FIGS. 10A-10C are schematics of molecular models showing the beneficial mutations depicted in each of FIGS. 9A, 9B and 9C, respectively; FIG. 10A depicts the structural changes of swapping the wild-type “GAAA” tetraloop to “GAGA” tetraloop, resulting in changing the conformation from U-turn to Z-turn; FIG. 10B depicts the effects of swapping the WT “GAAA” tetraloop to “GGUG”, which led to the “flipping” of G63, G64, and G65; and FIG. 10C depicts lengthening stem regions of stem-loop 2, (i.e., 3 bp extension), which brings the tetraloop closer to H721 and E722.

### DETAILED DESCRIPTION OF THE INVENTION

### I. Definitions

The terms “nucleic acid,” “nucleic acid sequence,” “nucleic acid fragment,” “oligonucleotide,” and “polynucleotide” are used interchangeably and are intended to include, but not limited to, a polymeric form of nucleotides that may have various lengths, either deoxyribonucleotides (DNA) or ribonucleotides (RNA), or analogs or modified nucleotides thereof, including, but not limited to locked nucleic acids (LNA), peptide nucleic acids (PNA), and morpholinos. An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non standard nucleotide(s), nucleotide analog(s) and/or modified nucleotides. In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some forms the set of characters is (A, C, G, T, U) for adenosine, cytidine, guanosine, thymidine, and uridine respectively. In some forms the set of characters is (A, C, G, T, U, I, X) for adenosine, cytidine, guanosine, thymidine, uridine, inosine, xanthosine, respectively. The modified sequences, non-natural sequences, or sequences with modified binding, may be in the genomic, the guide or the tracr sequences.

As used herein, the terms “percent (%) sequence identity,” or “% identical to (sequence)” are used interchangeably and are defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The terms “protein” “polypeptide” or “peptide” refer to a natural or synthetic molecule including two or more amino acids linked by the carboxyl group of one amino acid to the alpha amino group of another.

The term “polynucleotide” or “nucleic acid” or “nucleic acid sequence” refers to a natural or synthetic molecule including two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

A cell can be in vitro. Alternatively, a cell can be in vivo and can be found in a subject. A “cell” can be a cell from any organism including, but not limited to, a bacterium.

The terms “editing fidelity” or “editing efficiency” or “targeting accuracy” or “on-target editing” or “on-off target specificity” or “on-target editing efficiency” are understood to mean the percentage of desired mutation achieved and are measured by the precision of the sgRNA variant in altering the DNA construct of the targeted gene with minimal off-target editing. A DNA editing efficiency of 1 (or 100%) indicates that the number of edited cells and/or edited alleles obtained when the sgRNA variant is used is approximately equal or equal to the number of edited cells and/or edited alleles obtained when the wild type or parent sgRNA variant is used. Conversely, a DNA editing efficiency greater than 1 indicates that the number of edited cells obtained when the sgRNA variant used is greater than the number of edited cells obtained when the parent sgRNA variant is used. In this case, the sgRNA variant has improved properties, for example improved editing efficiency when compared to the parent sgRNA.

The terms “single guide RNA” or “sgRNA” refer to the polynucleotide sequence comprising the guide sequence, tracr sequence and the tracr mate sequence. “Guide sequence” refers to the around 20 base pair (bp) sequence within the guide RNA that specifies the target site and may be used interchangeably with the terms “guide” or “spacer.”

The term “stem-loop 2 region” refers to the polynucleotide sequence of the second hairpin structure of the sgRNA and the flanking sequence.”

The terms “genome editing,” “genome engineering” or “genome mutagenesis” refer to selective and specific changes to one or more targeted genes or DNA sequences within a recipient cell through programming of the CRISPR-Cas system within the cell. The editing or changing of a targeted gene or genome can include one or more of a deletion, knock-in, point mutation, substitution mutation or any combination thereof in one or more genes of the recipient cell.

The terms “vector” or “expression vector” refer to a system suitable for delivering and expressing a desired nucleotide or protein sequence. Some vectors may be expression vectors, cloning vectors, transfer vectors etc.

The term “variant” or “mutant,” as used herein refer to an artificial outcome that has a pattern that deviates from what occurs in nature. In the context of the disclosed sgRNA variants, “variant” refers to a sgRNA that has one or more nucleic acid changes in the scaffold region relative to wildtype sgRNA scaffold region (e.g., SEQ ID NO:345), or relative to a starting, base, or reference sgRNA, such as “E+F” (SEQ ID NO:346); “U61C/A66G” (SEQ ID NO:347); “U61C/A66G” (SEQ ID NO:348); “E+F G62A/A64G” (SEQ ID NO:349); “G62A/A64G” (SEQ ID NO:350); and “5E” (SEQ ID NO:351). Note that the disclosed sgRNA variants have one or nucleic amino acid changes relative to a reference, base, or starting sgRNA (such as, e.g., wildtype sgRNA or “E+F”; “U61C/A66G”; “U61C/A66G”; “E+F G62A/A64G”; “G62A/A64G”; and “5E”. While some such reference, base, or starting sgRNAs (such as, e.g., G62A/A64G) are themselves a “variant” of another or other sgRNA, these reference, base, or starting sgRNAs are not a disclosed variant as described herein, and reference herein to such reference, base, or starting sgRNAs as a “variant” sgRNA is not intended to, and does not, indicate that such reference, base, or starting sgRNAs are a disclosed variant that impart enhanced editing, as described herein.

The terms “Protospacer adjacent motif” or “PAM sequence” or “PAM interaction region” refer to short pieces of genetic code that flag editable sections of DNA and serve as a binding signal for specific CRISPR-Cas nucleases. The PAM interaction region in the wild-type SaCas9 or its variants contains amino acid residues 910-1053 (Nishimasu, et al. Cell, 162, 1113-1126, doi: 10.1016/j.cell.2015.08.007 (2016)) and includes a conserved 13-amino acid region spanning positions 982 to 994 which plays a role in binding to the 4th and 5th bases of the PAM (Ma, et al. Nature Communications, 10, 560, doi: 10.1038/s41467-019-08395-8 (2019)).

The terms “Cas9,” “Cas9 protein,” or “Cas9 nuclease” refer to a RNA-guided endonuclease that is a Cas9 protein that catalyzes the site-specific cleavage of double stranded DNA. Also, referred to as “Cas nuclease” or “CRISPR-associated nuclease.”

The term “mutation” refers to a substitution of a residue within a sequence, e.g., a nucleic acid or amino acid sequence, with another residue, or a deletion or insertion of one or more residues within a sequence. Mutations are described by identifying the original residue followed by the position of the residue within the sequence and by the identity of the change in residue. For the purposes of this disclosure, amino acid positions are identified using the amino acid positions shown in SpCas9 sequence UniProtKB/Swiss-Prot No. Q99ZW2 (PDB ID NO:600Y), with the numbering beginning at the initial methionine residue. Various methods for making the mutations in the amino acids provided herein are well known in the art, and are provided by, for example, Green and Sambrook, Molecular Cloning: A Laboratory Manual 4th Edition, Cold Spring Harbor Laboratory Press, (2012).

The use of the terms “a,” “an,” “the,” and similar referents in the context of describing the present disclosure (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the disclosure.

### II. CRISPR/Cas Systems with Enhanced Specificity

Variant guide RNA scaffolds that impart enhanced editing activity and high genome-wide targeting specificity in human cells have been developed. The engineered variant guide RNA scaffolds implement activity-enhancing mutations that enhance their editing activities as compared with wild-type guide RNA scaffolds and pre-existing variants. An advantage of the CRISPR-Cas system is that a single Cas protein can be programmed by guide molecules to recognize a specific nucleic acid target. In other words the CRISPR-Cas protein can be recruited to a specific nucleic acid target locus of interest using said guide molecule.

The term “CRISPR” (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). Methods of preparing compositions for use in genome editing using the CRISPR/Cas systems are described in detail in WO 2013/176772 and WO 2014/018423, which are specifically incorporated by reference herein in their entireties.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease. Typically, a CRISPR-Cas9 system includes a guide RNA (gRNA) and Cas9 nuclease, which together form a ribonucleoprotein (RNP) complex. The presence of a specific protospacer adjacent motif (PAM) in the genomic DNA is required for the gRNA to bind to the target sequence. The Cas9 nuclease then makes a double-strand break in the DNA. Endogenous repair mechanisms triggered by the double-strand break may result in gene knockout via a frameshift mutation or knock-in of a desired sequence if a DNA template is present.

In some forms, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

CRSIPR systems having enhanced editing activity and high genome-wide targeting specificity typically include two components: (1) a single guide RNA configured for enhanced editing activity; and (2) a Cas enzyme.

It has been established that engineering the activity of an enzyme and its working component (in this case the sgRNA scaffold for Cas9 enzyme) by introducing modifications to the component typically increases or decreases both the on-target and the off-target activities simultaneously. However, it has been established that the described sgRNA scaffold variants decrease undesired off-target activity while also increasing on-target activity at targeted genomic loci (e.g., HBG loci, as indicated in the Examples). Therefore, the described variants achieve accurate and efficient genome editing at any user-defined target.

In some forms, a variant single guide RNA (sgRNA) includes substitution and/or addition of one or more nucleic acid residues that strengthens the interaction of the sgRNA with a Cas enzyme,
whereby the strengthened interaction imparts increased on-target editing and/or increased on-off target specificity relative to a wild type sgRNA that lacks the substitution and/or addition of one or more nucleic acid residues.

In some forms, a variant single guide RNA (sgRNA) includes substitution and/or addition of one or more nucleic acid residues that strengthens the interaction of the sgRNA with a Cas enzyme, whereby the strengthened interaction imparts decreased off-target activity while also increasing on-target activity at a targeted genomic locus relative to a wild type sgRNA that lacks the substitution and/or addition of one or more nucleic acid residues.

A. Single Guide RNA (sgRNA)

The single guide RNA is a specific RNA sequence that recognizes the target DNA region of interest and directs the Cas nuclease there for editing. The gRNA is made up of two parts: CRISPR RNA (crRNA), a 17-20 nucleotide spacer sequence complementary to the target DNA and a conserved repeat fragment (“handle” or “tag”) region that pairs with the tracr RNA, and a tracr RNA, which serves as a binding scaffold for the Cas nuclease. The crRNA component imparts specificity of CRISPR-directed nuclease activity and is the customizable component that directs specific editing.

sgRNA is an abbreviation for “single guide RNA.” sgRNA is a single RNA molecule that contains both the custom-designed short crRNA sequence fused to the scaffold tracrRNA sequence. sgRNA is synthetically generated or made in vitro or in vivo from a DNA template.

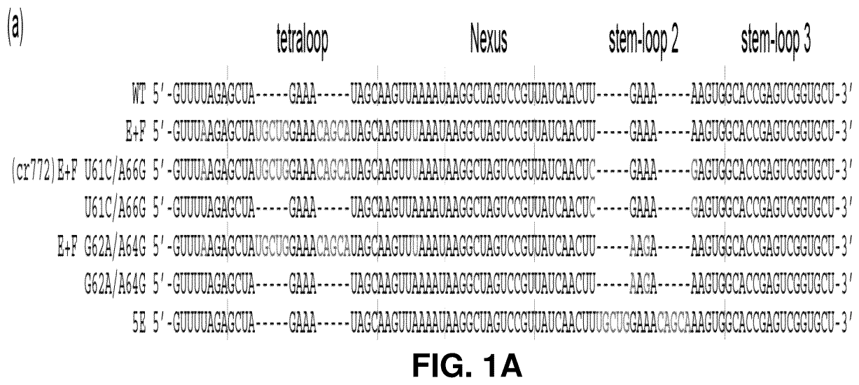

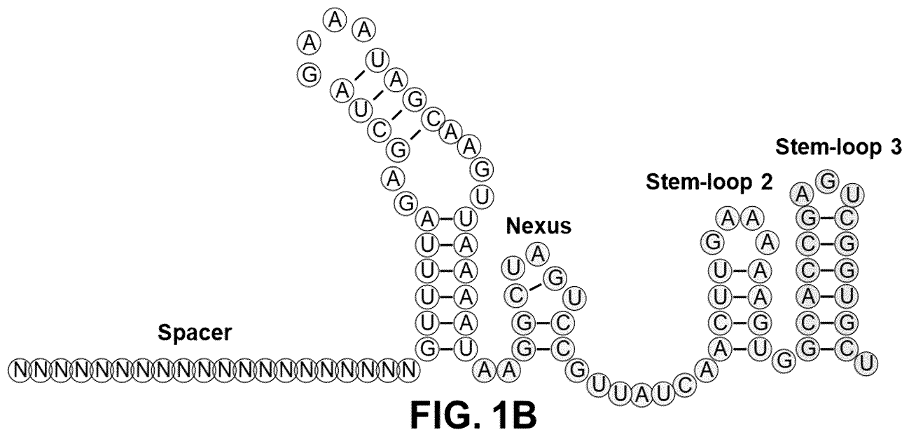

While crRNAs and tracrRNAs exist as two separate RNA molecules in nature, sgRNAs include both a crRNA component and a scaffold component fused as a single molecule. The nucleic acid sequence of the scaffold of a wildtype sgRNA appended with corresponding structural features is presented in FIG. 1.

In some forms, the nucleic acid sequence of a wild-type sgRNA scaffold sequence is:

(SEQ ID NO: 345)
GUUUUAGAGCUAGAAAUAGCAAGUUAAAAUAAGGCUAGUCCGUUAUCAAC
UUGAAAAAGUGGCACCGAGUCGGUGCU.

In the complete sgRNA, the guide sequence immediately precedes the first nucleotide of the tracr sequence. In some forms, the different regions of an sgRNA scaffold sequence are defined by the secondary structural elements formed within the sequence of scaffold RNA. For example, in some forms, the sgRNA scaffold sequence includes the structural elements set forth in FIG. 1A, indicated as the “tetraloop” region, the “nexus” region, the stem-loop 2 region and the stem-loop 3 region. These structural features are indicated on the schematic representation of an sgRNA set forth in FIG. 1B. In an exemplary form, when the sgRNA scaffold sequence has the structure of a wild-type sgRNA having a sequence of SEQ ID NO:345, the sgRNA scaffold sequence includes 77 nucleic acid residues, whereby nucleotides in positions 13-16 represent the “tetraloop” region; nucleotides in positions 31-43 represent the “nexus” region; 18 nucleotides in positions 44-61 represent the “stem-loop 2” region; and nucleotides in positions 62-77 represent the “stem-loop 3” region.

As described herein, the sgRNA scaffold stem-loop 2 region includes a hairpin region, as well as flanking regions. The flanking regions includes 6 nucleotides (i.e., at positions 44-48 of SEQ ID NO:345 and at position 61 of SEQ ID NO:345) and all other residues within the stem-loop 2 region form the “hairpin region”. For example, in the wild-type sgRNA scaffold having a sequence of SEQ ID NO:345, the flanking region includes nucleotides in positions 44-48 and 61, and the “hairpin region of stem-loop 2” includes 12 nucleotides in positions 49-60.

In some forms, the sgRNA scaffold sequence includes all components of a wild-type sgRNA directly preceding the stem-loop 2 region, and having the nucleic acid sequence:

(SEQ ID NO: 356)
GUUUUAGAGCUAGAAAUAGCAAGUUAAAAUAAGGCUAGUCCGU.

In some forms, the sgRNA scaffold sequence includes all components of a wild-type sgRNA directly preceding the hairpin region of stem-loop 2, and having the nucleic acid sequence:

(SEQ ID NO: 357)
GUUUUAGAGCUAGAAAUAGCAAGUUAAAAUAAGGCUAGUCCGUUAUCA.

In some forms, the sgRNA scaffold sequence includes all components of a wild-type sgRNA directly following the stem-loop 2 region, having the nucleic acid sequence: GCACCGAGUCGGUGCU (SEQ ID NO:358).

In some forms, the sgRNA scaffold sequence includes all components of the wild type sgRNA, but with the hairpin region of stem-loop 2 substituted. For example, in some forms, a sgRNA scaffold includes the sequence:

(SEQ ID NO: 354)
GUUUUAGAGCUAGAAAUAGCAAGUUAAAAUAAGGCUAGUCCGUUAUCA-
X-GGCACCGAGUCGGUGCU,

whereby “—X—” represents between 12 and 24 nucleic acid residues corresponding to a hairpin region of stem-loop 2.

An exemplary stem-loop 2 region of wild-type sgRNA scaffold is:

(SEQ ID NO: 359)
UAUCAACUUGAAAAAGUG,

whereby the hairpin region of stem-loop 2 includes the 12-nucleotide sequence:

(SEQ ID NO: 360)
ACUUGAAAAAGU.

1. Variant sgRNAs (sgRNA)

Multiple variant sgRNAs are known in the art to alter or otherwise mediate the editing activity of CRISPR/Cas relative to the Wt sgRNA. Exemplary variant sgRNAs that are known in the art include:

“E+F”, having a nucleic acid sequence of:

(SEQ ID NO: 346)
GUUUAAGAGCUAUGCUGGAAACAGCAUAGCAAGUUUAAAUAAGGCUAGUC
CGUUAUCAACUUGAAAAAGUGGCACCGAGUCGGUGCU;

“(CR772) E+F U61C/A66G”, having a nucleic acid sequence of:

(SEQ ID NO: 347)
GUUUAAGAGCUAUGCUGGAAACAGCAUAGCAAGUUUAAAUAAGGCUAGUC
CGUUAUCAACUCGAAAGAGUGGCACCGAGUCGGUGCU;

“U61C/A66G”, having a nucleic acid sequence of:

(SEQ ID NO: 348)
GUUUUAGAGCUAGAAAUAGCAAGUUAAAAUAAGGCUAGUCCGUUAUCAAC
UCGAAAGAGUGGCACCGAGUCGGUGCU;

“E+F G62A/A64G”, having a nucleic acid sequence of:

(SEQ ID NO: 349)
GUUUAAGAGCUAUGCUGGAAACAGCAUAGCAAGUUUAAAUAAGGCUAGUC
CGUUAUCAACUUAAGAAAGUGGCACCGAGUCGGUGCU;

“G62A/A64G”, having a nucleic acid sequence of:

SEQ ID NO: 350)
GUUUUAGAGCUAGAAAUAGCAAGUUAAAAUAAGGCUAGUCCGUUAUCAAC
UUAAGAAAGUGGCACCGAGUCGGUGCU;

and

“5E”, having a nucleic acid sequence of:

(SEQ ID NO: 351)
GUUUUAGAGCUAGAAAUAGCAAGUUAAAAUAAGGCUAGUCCGUUAUCAAC
UUUGCUGGAAACAGCAAAGUGGCACCGAGUCGGUGCU.

2. Variant sgRNAs Enhancing Editing

Variant sgRNAs that enhance the specificity and activity of the editing activity of CRISPR/Cas relative to the Wt sgRNA have been developed. In some forms, the variant sgRNAs enhance the specificity and activity of the editing activity of CRISPR/Cas relative to the Wt sgRNA by increasing the stability of the interaction with the Cas enzyme. Therefore, compositions of variant sgRNAs that have increased stability of the interaction with the Cas enzyme relative to the Wt sgRNA and which have enhanced specificity and activity of on-target editing activity of CRISPR/Cas relative to the Wt are described. Exemplary variant sgRNAs which have enhanced specificity and activity of on-target editing activity of CRISPR/Cas relative to the Wt include variants of the stem-loop 2 region.

Exemplary variant sgRNA scaffolds with enhanced specificity and activity of on-target editing activity of CRISPR/Cas relative to the Wt having variants of the hairpin region of stem-loop 2 are set forth in Table 4. Therefore, in some forms, the variant sgRNA scaffold with enhanced specificity and activity of on-target editing activity of CRISPR/Cas relative to the Wt includes a hairpin region of stem-loop 2 having a sequence of nucleic acids of any one of the sequences in Table 4. For example, in some forms, the variant sgRNA scaffold with enhanced specificity and activity of on-target editing activity of CRISPR/Cas relative to the Wt includes a hairpin region of stem-loop 2 having a sequence of nucleic acids of any one of SEQ ID NOs:1-312.

In some forms, the variant strengthens the scaffold's interaction with SpCas9 via His721 and the PI domain of SpCas9. For example, in some forms, the variant has a hairpin region of stem-loop 2 including the nucleic acid sequence:

(“SV48”; SEQ ID NO: 48)
GCGGGGUGCCGC.

In some forms, the variant has a hairpin region of stem-loop 2 including the nucleic acid sequence:

(“SV240”; SEQ ID NO: 240)
GGGCCGGGGUGCCGGCCC.

In some forms, the variant includes all or part of a “framework” sgRNA, such as that of the wild type sgRNA scaffold (residues corresponding to the stem-loop 2 region are in boldface):

(SEQ ID NO: 345)
GUUUUAGAGCUAGAAAUAGCAAGUUAAAAUAAGGCUAGUCCGU
UAUCAACUUGAAAAAGUGGCACCGAGUCGGUGCU.

In some forms, the variant of the sgRNA includes the entire wild type sgRNA scaffold, but with the hairpin region of stem-loop 2 substituted. Therefore, an exemplary sgRNA includes the sequence:

(SEQ ID NO: 354)
GUUUUAGAGCUAGAAAUAGCAAGUUAAAAUAAGGCUAGUCCGUUAUCA-
X-GGCACCGAGUCGGUGCU,

whereby “—X—” represents between 12 and 24 nucleic acid residues corresponding to a hairpin region of stem-loop 2. For example, in some forms, the variant of the sgRNA including the entire wild type sgRNA scaffold with the hairpin region of stem-loop 2 substituted includes the sequence:

(SEQ ID NO: 355)
GUUUUAGAGCUAGAAAUAGCAAGUUAAAAUAAGGCUAGUCCGUUAUCA
NNNNNNNNNNNNGGCACCGAGUCGGUGCU,

whereby each “—N—” independently represents either “A”, “U”, “C” or “G”, respectively.

In some forms, the sgRNA includes SEQ ID NO:354, whereby “—X—” represents any one of SEQ ID NOs: 1-312.

In some forms, the variant sgRNA includes a hairpin region of stem-loop 2 corresponding to SEQ ID NO:48. Therefore, in some forms, the variant sgRNA has a nucleic acid sequence of:

GUUUUAGAGCUAGAAAUAGCAAGUUAAAAUAAGGCUAGUCCGUUAUCAGC
GGGGUGCCGCGGCACCGAGUCGGUGCU (“sgRNA-48”; SEQ ID
NO: 352).

In some forms, the variant sgRNA includes a hairpin region of stem-loop 2 corresponding to SEQ ID NO:240. Therefore, in some forms, the variant sgRNA has a nucleic acid sequence of:

GUUUUAGAGCUAGAAAUAGCAAGUUAAAAUAAGGCUAGUCCGUUAUCAGG
GCCGGGGUGCCGGCCCGGCACCGAGUCGGUGCU (“sgRNA-240”;
SEQ ID NO: 353).

In other forms, the variant sgRNA includes a hairpin region of stem-loop 2 corresponding to a variant having at least 75%, up to 99% identity to SEQ ID NO:48 or SEQ ID NO:240.

The term “identity,” as used herein, can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). In some forms, the default parameters can be used to determine the identity for the polynucleotides of the present disclosure. In some forms, the % sequence identity of a given nucleic acid sequence “C” to, with, or against a given nucleic acid or amino acid sequence “D” (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides in D. It will be appreciated that where the length of sequence C is not equal to the length of sequence D, the % sequence identity of C to D will not equal the % sequence identity of D to C.

In other forms, the variant sgRNA includes a hairpin region of stem-loop 2 corresponding to a variant having at least 75%, up to 99% identity to GCGGGGUGCCGC (“SV48”; SEQ ID NO:48). For example in some forms, the variant has a hairpin region of stem-loop 2 corresponding to a variant having at least about 75%, 80%, 85%, 90%, or 95% identity to SEQ ID NO:48. Therefore, in some forms, the variant sgRNA has a hairpin region of stem-loop 2 with a nucleic acid sequence that has one or more nucleotides different to SEQ ID NO:48, such as one or more substitutions, deletions or additions at any one of the nucleotide positions of SEQ ID NO:48.

In some forms, the variant sgRNA includes a hairpin region of stem-loop 2 having one, two, three, four, or five residues that are substituted, deleted, or added relative to the 12 nucleotide sequence “GCGGGGUGCCGC” (“SV48”; SEQ ID NO:48). Therefore, a variant sequence having a substitution, deletion, or addition at any one of positions 1-12 will result in a variant having approximately 92% sequence identity to SEQ ID NO:48; a variant sequence having two mutations will result in a variant having approximately 83% sequence identity; a variant sequence having three mutations will result in a variant having approximately 75% sequence identity; a variant sequence having four mutations will result in a variant having approximately 66% sequence identity; and a variant sequence having five mutations will result in a variant having approximately 57% sequence identity to SEQ ID NO:48, respectively. Therefore, in some forms, the variant sgRNA includes a hairpin region of stem-loop 2 having at least about 56%, at least about 65%, at least about 74%, at least about 82%, or at least about 91% sequence identity to SEQ ID NO:48.

In other forms, the variant sgRNA includes a hairpin region of stem-loop 2 corresponding to a variant having at least 75%, up to 99% identity to

GGGCCGGGGUGCCGGCCC (“SV240”; SEQ ID NO: 240).

For example in some forms, the variant has a hairpin region of stem-loop 2 corresponding to a variant having at least 75%, 80%, 85%, 90%, 95% or 99% identity to SEQ ID NO:240.

Therefore, in some forms, the variant sgRNA has a hairpin region of stem-loop 2 nucleic acid sequence that has one or more nucleotides different to SEQ ID NO:240, such as one or more substitutions, deletions, or additions at any one of the nucleotide positions of SEQ ID NO:240. In some forms, the variant sgRNA includes a hairpin region of stem-loop 2 having one, two, three, four, five, or six residues that are substituted, deleted, or added relative to the 18 nucleotide sequence GGGCCGGGGUGCCGGCCC (“SV240”; SEQ ID NO:240). A variant sequence having a mutation (i.e., substitution, deletion, addition) of a single nucleotide at any one position (1-18) will result in a variant having approximately 94% sequence identity to SEQ ID NO:240; a variant sequence having two mutations will result in a variant having approximately 89% sequence identity; a variant sequence having three mutations will result in a variant having approximately 83% sequence identity; a variant sequence having four mutations will result in a variant having approximately 78% sequence identity; a variant sequence having five mutations will result in a variant having approximately 72% sequence identity; and a variant sequence having six mutations will result in a variant having approximately 66% sequence identity to SEQ ID NO:240, respectively. Therefore, in some forms, the variant sgRNA includes a hairpin region of stem-loop 2 having at least about 65%, at least about 71%, at least about 77%, at least about 82%, at least about 88%, or at least 94% sequence identity to SEQ ID NO:240.

In other forms, the framework region of the sgRNA scaffold is not that of the Wt sgRNA scaffold. For example, in some forms, the framework region of the sgRNA scaffold is derived from a variant sgRNA. Exemplary variant sgRNAs are known in the art, for example, including “E+F” (SEQ ID NO:346); “(CR772) E+F U61C/A66G” (SEQ ID NO:347); “U61C/A66G” (SEQ ID NO:348); “E+F G62A/A64G” (SEQ ID NO:349); “G62A/A64G” (SEQ ID NO:350); and “5E” (SEQ ID NO:351).

In some forms, the editing activity and specificity of the described variant sgRNAs including one or more mutations of the stem-loop 2 region is enhanced compared to that of the “Wild Type” (WT) sgRNA that does not include the mutations of the stem-loop 2 region. For example, in some forms, the described variant sgRNAs have increased on-target specificity compared to that of the “Wild Type” (WT) sgRNA that does not include the mutations of the stem-loop 2 region. Typically, when the described variant sgRNAs including one or more mutations of the stem-loop 2 region have increased specificity and editing activity of CRISPR/Cas as compared to that of the “Wild Type” (WT) sgRNA that does not include the mutations of the stem-loop 2 region, the described variant sgRNAs do not have increased off-target activity. In some forms, the described variant sgRNAs have decreased off-target activity compared to that of the “Wild Type” (WT) sgRNA that does not include the mutations of the stem-loop 2 region. In some forms, the described variant sgRNAs have increased on-target specificity and decreased off-target activity compared to that of the “Wild Type” (WT) sgRNA that does not include the mutations of the stem-loop 2 region.

In some forms, the described variant sgRNAs have increased on-target specificity of between about 1% and about 100%, inclusive, compared to that of the “Wild Type” (WT) sgRNA that does not include the mutations of the stem-loop 2 region. For example, in some forms, the described variant sgRNAs have increased on-target specificity of about 1%, 2%, 3%, 4%, 5%, 6%, 7%, 8%, 9%, 10%, 15%, 20%, 25%, 30%, 40%, 50%, 60%, 70%, 80%, 90%, 99% or about 100%, or more, as compared to that of the “Wild Type” (WT) sgRNA that does not include the mutations of the stem-loop 2 region. In some forms, the described variant sgRNAs have decreased off-target activity that is between about 1% and about 99% inclusive of that of the “Wild Type” (WT) sgRNA that does not include the mutations of the stem-loop 2 region. For example, in some forms, the described variant sgRNAs have decreased off-target activity that is only about 1%, 2%, 3%, 4%, 5%, 6%, 7%, 8%, 9%, 10%, 15%, 20%, 25%, 30%, 40%, 50%, 60%, 70%, 80%, 90%, up to about 99%, of that of the “Wild Type” (WT) sgRNA that does not include the mutations of the stem-loop 2 region.

In some forms, the described variant sgRNAs have increased on-target specificity of between about 1% and about 100% inclusive compared to that of the “Wild Type” (WT) sgRNA that does not include the mutations of the stem-loop 2 region, and decreased off-target activity that is between about 1% and about 99% inclusive of that of the “Wild Type” (WT) sgRNA that does not include the mutations of the stem-loop 2 region. For example, in some forms, the described variant sgRNAs have increased on-target specificity of about 1%, 2%, 3%, 4%, 5%, 6%, 7%, 8%, 9%, 10%, 15%, 20%, 25%, 30%, 40%, 50%, 60%, 70%, 80%, 90%, 99% or about 100%, or more, as compared to that of the “Wild Type” (WT) sgRNA that does not include the mutations of the stem-loop 2 region, and have decreased off-target activity that is only about 1%, 2%, 3%, 4%, 5%, 6%, 7%, 8%, 9%, 10%, 15%, 20%, 25%, 30%, 40%, 50%, 60%, 70%, 80%, 90%, up to about 99%, of that of the “Wild Type” (WT) sgRNA that does not include the mutations of the stem-loop 2 region.

### B. Cas Enzymes

Systems including Cas enzymes are provided. The CRISPR-associated Cas nuclease protein is a non-specific endonuclease. It is directed to the specific DNA locus by a gRNA, where it makes a double-strand break. There are several versions of Cas nucleases isolated from different bacteria. The most commonly used one is the Cas9 nuclease from Streptococcus pyogenes (SpCas9).

As used herein, the term “Cas” generally refers to an effector protein of a CRISPR Cas system or complex. The term “Cas” may be used interchangeably with the terms “CRISPR” protein, “CRISPR Cas protein,” “CRISPR effector,” CRISPR Cas effector,” “CRISPR enzyme,” “CRISPR Cas enzyme” and the like, unless otherwise apparent. The Crispr-Cas effector protein may be without limitation a type II, type V, or type VI Cas effector protein. Non-limiting examples of Crispr-Cas effector proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologues thereof, or modified versions thereof. In some forms, the CRISPR enzyme has DNA cleavage activity.

### 1. Cas9

In some forms, the Type II CRISPR enzyme is a Cas9 enzyme. The signature Cas9 effector proteins are large multi-domain RNA-dependent endonucleases that locate, bind, and cleave the double-stranded DNA (dsDNA) targets which are complementary to their guide RNAs. For recognition and binding to target DNA, Cas9 requires the protospacer adjacent motif (PAM), as a short conserved sequence located just downstream of the non-complementary strand of the target dsDNA. Recognition of the PAM (5′NGG3′) triggers dsDNA melting, enabling crRNA strand invasion and base pairing. The dsDNA cleavage mediation happens via the activity of separate HNH and RuvC nuclease domains. Also, Cas9 is a member of a small subset of Cas effectors that need a second trans-acting crRNA (tracrRNA) for gRNA processing and DNA cleavage.

Exemplary Cas9 enzymes are disclosed in International Patent Application Publication No. WO/2014/093595. In some forms, the Cas9 enzyme is S. pneumoniae, S. pyogenes or S. thermophilus Cas9, and may include mutated Cas9 derived from these organisms. The enzyme may be a Cas9 homolog or ortholog. Additional orthologs include, for example, Cas9 enzymes from Corynebacter diptheriae, Eubacterium ventriosum, Streptococcus pasteurianus, Lactobacillus farciminis, Sphaerochaeta globus, Azospirillum B510, Gluconacetobacter diazotrophicus, Neisseria cinereal, Roseburia intestinalis, Parvibaculum lavamentivorans, Staphylococcus aureus, Nitratifractor salsuginis DSM 16511, Camplyobacter lari CF89 12, and Streptococcus thermophilus LMD 9.

In some forms, the Cas9 effector protein and orthologs thereof may be modified for enhanced function. For example, improved target specificity of a CRISPR Cas9 system may be accomplished by approaches that include, but are not limited to, designing and preparing guide RNAs having optimal activity, selecting Cas9 enzymes of a specific length, truncating the Cas9 enzyme making it smaller in length than the corresponding wild-type Cas9 enzyme by truncating the nucleic acid molecules coding therefor and generating chimeric Cas9 enzymes wherein different parts of the enzyme are swapped or exchanged between different orthologs to arrive at chimeric enzymes having tailored specificity.

A Cas9 enzyme may include one or more mutations and may be used as a generic DNA binding protein with or without fusion to or being operably linked to a functional domain. The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains. Preferred examples of suitable mutations are the catalytic residue(s) in the N term RuvC I domain of Cas9 or the catalytic residue(s) in the internal HNH domain.

Generally, the Cas9 is (or is derived from) the Streptococcus pyogenes Cas9 (SpCas9). In such forms, preferred mutations are at any or all of positions 10, 762, 840, 854, 863 and/or 986 of SpCas9 or corresponding positions in other Cas9 orthologs with reference to the position numbering of SpCas9 (which may be ascertained for instance by standard sequence comparison tools, e.g. ClustalW or MegAlign by Lasergene 10 suite). In particular, any or all of the following mutations are preferred in SpCas9: D10A, E762A, H840A, N854A, N863A and/or D986A; as well as conservative substitution for any of the replacement amino acids is also envisaged. The same mutations (or conservative substitutions of these mutations) at corresponding positions with reference to the position numbering of SpCas9 in other Cas9 orthologs are also preferred. Particularly preferred are D10 and H840 in SpCas9. However, in other Cas9s, residues corresponding to SpCas9 D10 and H840 are also preferred. These are advantageous as when singly mutated they provide nickase activity and when both mutations are present the Cas9 is converted into a catalytically null mutant which is useful for generic DNA binding.

In some forms, chimeric Cas9 proteins are used. Chimeric Cas9 proteins are proteins that include fragments that originate from different Cas9 orthologs. For instance, the N terminal of a first Cas9 ortholog may be fused with the C terminal of a second Cas9 ortholog to generate a resultant Cas9 chimeric protein. These chimeric Cas9 proteins may have a higher specificity or a higher efficiency than the original specificity or efficiency of either of the individual Cas9 enzymes from which the chimeric protein was generated. These chimeric proteins may also include one or more mutations or may be linked to one or more functional domains. Also suitable are Cas9 proteins that have different PAM specificities. Typically, Cas9 proteins, such as Cas9 from S. pyogenes (spCas9), require a canonical NGG PAM sequence to bind a particular nucleic acid region.

Cas9 nuclease sequences and structures are known to those of skill in the art (Ferretti, et al. Proc Natl Acad Sci U.S.A, 98, 4658-4863, doi: 10.1073/pnas.071559398 (2001); Deltcheva, et al. Nature, 471, 602-607, doi: 10.1038/nature09886 (2011)). Cas9 orthologs have been described in several species of bacteria, including but not limited to Streptococcus pyogenes and Streptococcus thermophilus, Campylobacter jejuni and Neisseria meningitidis. (Slaymaker, et al. Science, 351, 84-88 doi: 10.1126/science.aad5227 (2016); Kleinstiver, et al. Nature, 529, 490-495, doi: 10.1038/nature 16526 (2016); Chen, et al. Nature, 550, 407-410, doi: 10.1038/nature24268 (2017); Casini, et al. Nat Biotechnol, 6, 265-271, doi: 10.1038/nbt.4066 (2018); Lee, et al. Nat Commun, 9, 3048, doi: 10.1038/s41467-018-05477-x (2018); Vakulskas, et al. Nat Med, 24, 1216-1224, doi: 1.1038/s41591-018-0137-0 (2018); Choi, et al. Nat Methods, 16, 722-730, doi: 10.1038/s41592-019-0473-0 (2019); Kim, et al. Nat Commun, 8, 14500, doi: 10.1038/ncomms14500 (2017); (Edraki, et al. Mol Cell, 73, 714-726, doi: (2019)).

### C. Ribonucleoprotein Complexes

Enhanced ribonucleoprotein complexes including a Cas enzyme and one of the described variant sgRNAs are also provided. Typically, the enhanced ribonucleoprotein complexes have enhanced specificity and activity of on-target editing activity of CRISPR/Cas relative to the ribonucleoprotein complex formed by association of the same Cas enzyme with a Wt sgRNA. In some forms, an enhanced ribonucleoprotein complex includes:
(i) a Cas enzyme; and
(ii) a variant sgRNAs with enhanced specificity and activity of on-target editing activity of CRISPR/Cas relative to Wt sgRNA,
whereby the Cas enzyme and the variant sgRNA are bound together with greater affinity than relative to a the complex between a Wt sgRNA and the same Cas enzyme. Typically the Cas enzyme is a Cas9 enzyme. Typically, the Cas9 enzyme is derived from S. pyogenes (spCas9).

In some forms, the ribonucleoprotein complex includes a variant sgRNA including a stem-loop 2 region set forth in Table 4. Therefore, in some forms, the ribonucleoprotein complex with enhanced specificity and activity of on-target editing activity of CRISPR/Cas relative to the Wt includes a variant sgRNA a stem-loop 2 region having a sequence of nucleic acids of any one of the sequences in Table 4. For example, in some forms, the ribonucleoprotein complex with enhanced specificity and activity of on-target editing activity of CRISPR/Cas relative to the Wt includes a variant sgRNA having a stem-loop 2 region formed from a sequence of nucleic acids of any one of SEQ ID NOs: 1-312.

In some forms, the variant strengthens the scaffold's interaction with SpCas9 via His721 and the PI domain of SpCas9. For example, in some forms, the ribonucleoprotein complex with enhanced specificity and activity of on-target editing activity of CRISPR/Cas relative to the Wt includes a variant sgRNA stem-loop 2 region having a nucleic acid sequence: GCGGGGUGCCGC (“SV48”; SEQ ID NO:48). In other forms, the ribonucleoprotein complex with enhanced specificity and activity of on-target editing activity of CRISPR/Cas relative to the Wt includes a variant sgRNA stem-loop 2 region having a nucleic acid sequence: GGGCCGGGGUGCCGGCCC (“SV240”; SEQ ID NO:240).

In some forms, the ribonucleoprotein complex with enhanced specificity and activity of on-target editing activity of CRISPR/Cas relative to the Wt includes a Cas9 enzyme and a variant sgRNA having a nucleic acid sequence of:

GUUUUAGAGCUAGAAAUAGCAAGUUAAAAUAAGGCUAGUCCGUUAUCAGC
GGGGUGCCGCGGCACCGAGUCGGUGCU (“sgRNA-48”; SEQ ID
NO: 352).

In other forms, the ribonucleoprotein complex with enhanced specificity and activity of on-target editing activity of CRISPR/Cas relative to the Wt includes a Cas9 enzyme and a variant sgRNA having a nucleic acid sequence of:

GUUUUAGAGCUAGAAAUAGCAAGUUAAAAUAAGGCUAGUCCGUUAUCAGG
GCCGGGGUGCCGGCCCGGCACCGAGUCGGUGCU (“sgRNA-240”;
SEQ ID NO: 353).

### III. Methods of Use

Methods for using the described compositions for enhanced gene editing are described. The described variant sgRNAs and ribonucleoprotein complexes thereof can be used for any suitable purpose and in any suitable method for CRISPR-based editing of DNA.

Generally, the disclosed variants can be used to cleave target DNA of interest. Such cleavage is preferably used in a method of editing the target DNA of interest. For example, the disclosed variants can be used for and in any known methods of DNA editing, including in vitro and in vivo DNA editing. sgRNAs, of which the disclosed variants are new forms, can be and have been used for various DNA cleavage and editing methods and the disclosed variants can be used as the RNA-guided endonuclease in any of these methods uses. For example, the disclosed variants can be used for altering the genome of a cell. Various methods for selectively altering the genome of a cell using RNA-guided endonucleases are described in the following exemplary U.S. Patent documents: U.S. Pat. Nos. 8,993,233, 9,023,649, and 8,697,359 and U.S. Patent Application Publication Nos. 20140186958, 20160024529, 20160024524, 20160024523, 20160024510, 20160017366, 20160017301, 20150376652, 20150356239, 20150315576, 20150291965, 20150252358, 20150247150, 20150232883, 20150232882, 20150203872, 20150191744, 20150184139, 20150176064, 20150167000, 20150166969, 20150159175, 20150159174, 20150093473, 20150079681, 20150067922, 20150056629, 20150044772, 20150024500, 20150024499, 20150020223, 20140356867, 20140295557, 20140273235, 20140273226, 20140273037, 20140189896, 20140113376, 20140093941, 20130330778, 20130288251, 20120088676, 20110300538, 20110236530, 20110217739, 20110002889, 20100076057, 20110189776, 20110223638, 20130130248, 20150050699, 20150071899, 20150050699, 20150045546, 20150031134, 20150024500, 20140377868, 20140357530, 20140349400, 20140335620, 20140335063, 20140315985, 20140310830, 20140310828, 20140309487, 20140304853, 20140298547, 20140295556, 20140294773, 20140287938, 20140273234, 20140273232, 20140273231, 20140273230, 20140271987, 20140256046, 20140248702, 20140242702, 20140242700, 20140242699, 20140242664, 20140234972, 20140227787, 20140212869, 20140201857, 20140199767, 20140189896, 20140186958, 20140186919, 20140186843, 20140179770, 20140179006, 20140170753, and 20150071899, each of which is incorporated by reference herein, and in particular for their description of the uses of RNA-guided endonucleases.

Various methods for selectively altering the genome of a cell using RNA-guided endonucleases are described in the following exemplary publications: WO 2014/099744; WO 2014/089290; WO 2014/144592; WO 2014/004288; WO 2014/204578; WO 2014/152432; WO 2015/099850; WO 2008/108989; WO 2010/054108; WO 2012/164565; WO 2013/098244; WO 2013/176772; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

Disclosed are methods of editing a sequence of interest. In some forms, the method includes contacting a disclosed construct with the host of interest, where the host of interest harbors the sequence of interest and where the cell expresses a construct to produce the variant sgRNA and a Cas9 enzyme. In some forms, the method includes contacting a disclosed construct with the host of interest, where the host of interest harbors a sequence of interest and where the cell expresses the construct to produce the variant. In some forms, the method includes contacting the sequence of interest with a disclosed mixture, whereby the variant edits the sequence of interest targeted by the sgRNA.

In some forms, the method can further includes causing a variant sgRNA targeting the sequence of interest to be present in the host of interest with the produced variant, whereby the produced variant edits the sequence of interest targeted by the sgRNA.

The description can be further understood by reference to the following numbered paragraphs:

1. A variant single guide RNA (sgRNA) including substitution and/or addition of one or more nucleic acid residues that strengthens the interaction of the sgRNA with a Cas enzyme,
wherein the strengthened interaction imparts increased on-target editing and/or increased on-off target specificity relative to a wild type sgRNA that lacks the substitution and/or addition of one or more nucleic acid residues.

2. The variant sgRNA of paragraph 1, wherein the substitution and/or addition of one or more nucleic acid residues that strengthens the interaction of the sgRNA with a Cas enzyme includes substitution and/or addition of one or more nucleic acid residues within the hairpin region of the stem-loop 2 of the sgRNA.

3. The variant sgRNA of paragraph 1 or 2, wherein the Cas enzyme is a Cas9 enzyme.

4. The variant sgRNA of paragraph 3, wherein the Cas9 enzyme is derived from Streptococcus pyogenes (spCas9).

5. The variant sgRNA of paragraph 4, wherein the substitution and/or addition of one or more nucleic acid residues strengthens the sgRNAs interaction with residue His721 and/or the PI domain of SpCas9.

6. The variant sgRNA of any one of paragraphs 2-5, including the nucleic acid sequence:

(SEQ ID NO: 355)
GUUUUAGAGCUAGAAAUAGCAAGUUAAAAUAAGGCUAGUCCGUUAUCA-
X-GGCACCGAGUCGGUGCU,

wherein “—X—” represents a hairpin region of stem-loop 2 including between 12 and 24 nucleic acid residues, inclusive.

7. The variant sgRNA of any one of paragraphs 2-6, wherein the hairpin region of stem-loop 2 includes the nucleic acid sequence of any one of SEQ ID NOS: 1-312.

8. The variant sgRNA of any one of paragraphs 2-7, wherein the hairpin region of stem-loop 2 includes the nucleic acid sequence GCGGGGUGCCGC (SEQ ID NO:48), or a nucleic acid sequence having at least about 74% identity to SEQ ID NO:48.

9. The variant sgRNA of paragraph 8, wherein the hairpin region of stem-loop 2 includes a nucleic acid sequence having at least 82%, or at least 91% sequence identity to GCGGGGUGCCGC (SEQ ID NO:48).

10. The variant sgRNA of any one of paragraphs 2-7, wherein the hairpin region of stem-loop 2 includes the nucleic acid sequence GGGCCGGGGUGCCGGCCC (SEQ ID NO:240), or a nucleic acid sequence having at least about 75% identity to SEQ ID NO:240.

11. The variant sgRNA of paragraph 10, wherein the hairpin region of stem-loop 2 includes a nucleic acid sequence having at least 77%, at least 82%, at least 88%, or at least 94% sequence identity to GGGCCGGGGUGCCGGCCC (SEQ ID NO:240).

12. A variant sgRNA including a nucleic acid sequence of GUUUUAGAGCUAGAAAUAGCAAGUUAAAAUAAGGCUAGUCCGUUAUCAGCGG GGUGCCGCGGCACCGAGUCGGUGCU (SEQ ID NO:352), or a nucleic acid sequence having at least 75% identity to SEQ ID NO:352.

13. The variant sgRNA of paragraph 12, wherein the sgRNA includes a nucleic acid sequence having at least 80%, at least 85%, at least 90%, or at least 95% sequence identity to SEQ ID NO:352.

14. A variant sgRNA including a nucleic acid sequence of GUUUUAGAGCUAGAAAUAGCAAGUUAAAAUAAGGCUAGUCCGUUAUCAGGGC CGGGGUGCCGGCCCGGCACCGAGUCGGUGCU (SEQ ID NO:353), or a nucleic acid sequence having at least 75% identity to SEQ ID NO:353.

15. The variant sgRNA of paragraph 14, wherein the sgRNA includes a nucleic acid sequence having at least 80%, at least 85%, at least 90%, or at least 95% sequence identity to SEQ ID NO:353.

16. A ribonucleoprotein complex including:
(a) a Cas9 enzyme; and
(b) a variant sgRNA,
wherein the variant sgRNA includes a hairpin region of stem-loop 2 including the nucleic acid sequence of any one of SEQ ID NOs:1-312,
wherein the ribonucleoprotein complex has increased on-target editing and/or increased on-off target specificity relative to the corresponding complex between a Cas9 enzyme and wild type sgRNA.

17. The ribonucleoprotein complex of paragraph 16, wherein the Cas9 enzyme is derived from Streptococcus pyogenes (spCas9).

18. The ribonucleoprotein complex of paragraph 16 or 17, wherein the variant sgRNA includes the nucleic acid sequence:

(SEQ ID NO: 355)
GUUUUAGAGCUAGAAAUAGCAAGUUAAAAUAAGGCUAGUCCGUUAUCA-
X-GGCACCGAGUCGGUGCU,

wherein “—X—” represents a hairpin region of stem-loop 2 including the nucleic acid sequence of any one of SEQ ID NOs: 1-312.

19. The ribonucleoprotein complex of paragraph 18 including the sgRNA of any one of paragraphs 12 to 15.

20. A vector encoding of expressing the sgRNA of any one of paragraphs 1 to 15.

21. A cell including the sgRNA of any one of paragraphs 1 to 15, or the ribonucleoprotein complex of any one of paragraphs 16-19.

22. A method for CRISPR editing of one or more target genes in a cell, the method including administering into and/or expressing within the cell the ribonucleoprotein complex of any one of paragraphs 16-19,
wherein the ribonucleoprotein complex is configured to target the one or more target genes.

23. The method of paragraph 22, wherein the administering is in vivo.

24. A kit including
(i) the sgRNA of any one of paragraphs 1 to 15; and optionally
(ii) a Cas9 enzyme, or vector encoding or expressing the Cas9 enzyme; and/or
(iii) instructions for performing the method of paragraph 22. The present description is further illustrated by the following non-limiting examples. The entire contents of all of the references (including literature references, issued patents, published patent applications, and co-pending patent applications) cited throughout this application are hereby expressly incorporated by reference.

### EXAMPLES

### Example 1: Previously Described sgRNA Scaffold Variants with Improved On-Target Activity Exhibit Increased Off-Target Activity

The on- and off-target editing activities for SpCas9 nuclease using two published engineered sgRNA scaffold variants, E+F scaffold and cr772 (Chen, et al., Cell 2013, 155, (7), 1479-91; and Jost, et al., Nat Biotechnol 2020, 38, (3), 355-364) were evaluated.

### Methods

### Guide RNA Scaffold Library Design

PDB 600Y was used as the template for molecular modelling to simulate the likely consequences of stem-loop 2 lengthening. Variant sequences were submitted with 2-6 bp lengthening at the upper stem-loop 2 region to the ModeRNA server (available on the world wide web at “//iimcb.genesilico.pl/modernaserver/”) to generate threading models of the sgRNA scaffold and examined the sgRNA-SpCas9 protein interactions using UCSF Chimera v 1.14. ModeRNA was also used to generate sgRNA models containing the beneficial mutations previously reported (Jost, et al., Nat Biotechnol 2020, 38, (3), 355-364)) on nucleotides base-pair stack 58-69, 60-67, 61-66, to evaluate whether these mutations brought about fundamental structural changes in the protein models; no detrimental alterations generated by those mutations in the sgRNA scaffolds were identified. To generate a library of sgRNA scaffold variants focusing on the stem-loop 2 regions, RNA designer was used (available on the world wide web at “masoft.ca/cgi-bin/RNAsoft/RNAdesigner/rnadesign.pl”) with parameters: temperature: 37° ° C., target GC %: 50%, and allowing 10 designs. Only stem-loop 2 (position 54-70) was input to reduce computing time; the top design(s) with the minimum free energy was selected. Designs that fit with the U61G-A66C beneficial mutations were also filtered. In other words, the base pairing closest to the “AGAG” tetraloop was fixed to be G-C or C-G. Two versions of the stem-loop 2 lengthening scheme, the proximal (inserted at 61-66 base pair) and the distal (inserted at 58-69 base-pair) to the tetraloop were tested. Stem-length combinations for stem-loop2 (2-6 bp) extend between 61-66, and “GAAA” tetraloop, showing only the base pair 5′-3′ after 61 bp:

| | |
|---|---|
| 2 | AG |
| 3 | CUG |
| 4 | GCAG |
| 5 | UGCUG/CGUGC/GGCGG |
| 6 | GGUGCC/GACGCC |

Stem-length combinations for stem-loop2 (2-6 bps), extend between 58-69 and 59-68 base-pair, showing only the base pair sequence 5′-3′ after 58 bp:

| | |
|---|---|
| 2 | CC |
| 3 | CGG/CGC |
| 4 | CUGG |
| 5 | CCCCG |
| 6 | CCCGGU |

### Construction of DNA Vectors and Screen Library

The DNA vectors used (Table 1) were generated by standard molecular cloning strategies, including PCR, restriction enzyme digestion, oligo annealing and 5′ end phosphorylation, and ligation. Custom oligonucleotides were purchased from Genewiz. Vectors were transformed into E. coli strain DH5α competent cells and selected with ampicillin (100 mg/ml, USB) or carbenicillin (50 mg/ml, Teknova). Plasmid DNA was extracted and purified by Plasmid Mini (Takara) or Midi preparation (QIAGEN) kits. Sequences of the vectors were verified by Sanger sequencing.

sgRNA scaffold E+F in vector pJHp3 was generated by overlapping PCR of primers SY1, SY2, J15, and J16. The same strategy was used to obtain 5E in pJHp13, with the primers SY3, SY4, J17, and J18. The sequences of G62A/A64G in pJHp5 and E+F G62A/A64G in pJHp11 were PCR amplified by primers SA82 and J13 from pAWp28 and pJHp3, respectively. Similarly, the sequences of U61C/A66G in pJHp6 and cr772 in pJHp12 were PCR amplified by primers SA82 and J14 from pA Wp28 and pJHp3, respectively. All the PCR products were digested by XhoI and BamHI and inserted between the same sites in pAWp28 (Addgene, 73850) to generate the vectors. To construct the reporter vector pPZp257 for gene knockout and base editing, the mutant hU6 promoter was firstly PCR-amplified by primer pair Z350 and Z352 from pAWp28 and inserted between the SbfI and BamHI sites in pAWp9 (Addgene, 73851). Then an artificial reporter sequence (5′-3′):

(SEQ ID NO: 313)
ACCGGTCGTCTCCTTTTTTATCGTTTCCGCTTAACGGCGAAACGGTACGA
CAGCGTGTGCGGACAAGGCAAGGCTTGACCGACAATTGGAAGACTCCTAT
CCGTCAACGGAGACCAGATCTGGATGTTCCGGAGCTCCGGTACCAAATTG
CATGAAGCCAAGGCTCACGATCGGTGATGGGGATCC,

was synthesized and placed downstream of the hU6 promoter, leaving two Esp3I sites in between for sgRNA and scaffold insertion. A nicking sgRNA expression cassette, mutant mU6-dummysg2-v2 sgRNA scaffold, from pPZp138-3-4D (Guschin, et al., Methods Mol Biol 2010, 649, 247-56) was inserted downstream of the reporter. To generate the sgRNA scaffold variants library vector pPZp284a, a sgRNA targeting the reporter region and a truncated scaffold with two Esp3I sites for library insertion were annealed by two pairs of oligos Z518/Z519 and Z520/Z521 and ligated into the Esp3I sites in pPZp257. An array of oligo pairs containing 312 unique sgRNA scaffold sequences was synthesized. The oligo pairs were annealed and then cloned into the vector in pooled fashion. Sixty-fold representation of the library size was achieved in the cloning to ensure the coverage. pJF60b was generated by inserting a PCR amplified fragment from pCMV_AncBE4max_P2A_GFP (Addgene, 112100) into a lentiviral vector. Sequences of the primers and sgRNA spacer sequences used are listed in Table 2 and Table 3.

TABLE 1
Vectors

| Construct | |
|---|---|
| ID | Design |
| JHp40 | pFUGW-UBCp-RFPCMVp-GFP-U6p-RFPsg5-ON-E + F scaffold |
| JHp43 | pFUGW-UBCp-RFPCMVp-GFP-U6p-RFPsg5-ON-G62A/A64G |
| | scaffold |
| JHp44 | pFUGW-UBCp-RFPCMVp-GFP-U6p-RFPsg5-ON-U61C/A66G |
| | scaffold |
| JHp46 | pFUGW-UBCp-RFPCMVp-GFP-U6p-RFPsg5-ON-E + F |
| | G62A/A64G scaffold |
| JHp47 | pFUGW-UBCp-RFPCMVp-GFP-U6p-RFPsg5-ON-E + F |
| | U61C/A66G scaffold |
| JHp69 | pFUGW-UBCp-RFPCMVp-GFP-U6p-RFPsg5-ON-5E scaffold |
| KMp100 | pFUGW-CMVp-GFP-U6p-HPRTsg-E + F U61C/A66G scaffold |
| KMp101 | pFUGW-CMVp-GFP-U6p-HPRTsg-5E scaffold |
| KMp109 | pFUGW-CMVp-GFP-U6p-FANCFsg site 3-E + F G62A/A64G |
| | scaffold |
| KMp110 | pFUGW-CMVp-GFP-U6p-FANCFsg site 3-E + F U61C/A66G |
| | scaffold |
| KMp111 | pFUGW-CMVp-GFP-U6p-FANCFsg site 3-5E scaffold |
| KMp17 | pFUGW-hUbC-RFP-CMVp-GFP-U6p-RFPsg5-OFF5-2-WT |
| | scaffold |
| KMp19 | pFUGW-hUbC-RFP-CMVp-GFP-U6p-RFPsg5-OFF5-2-E + F |
| | U61C/A66G scaffold |
| KMp20 | pFUGW-hUbC-RFP-CMVp-GFP-U6p-RFPsg5-OFF5-2-5E scaffold |
| KMp73 | pFUGW-CMVp-GFP-U6p-FANCFsg site 6-E + F scaffold |
| KMp74 | pFUGW-CMVp-GFP-U6p-FANCFsg site 6-E + F G62A/A64G |
| | scaffold |
| KMp75 | pFUGW-CMVp-GFP-U6p-FANCFsg site 6-E + F U61C/A66G |
| | scaffold |
| KMp76 | pFUGW-CMVp-GFP-U6p-FANCFsg site 6-5E scaffold |
| KMp83 | pFUGW-CMVp-GFP-U6p-EMX1sg site 3-E + F scaffold |
| KMp84 | pFUGW-CMVp-GFP-U6p-EMX1sg site 3-E + F G62A/A64G |
| | scaffold |
| KMp85 | pFUGW-CMVp-GFP-U6p-EMX1sg site 3-E + F U61C/A66G |
| | scaffold |
| KMp86 | pFUGW-CMVp-GFP-U6p-EMX1sg site 3-5E scaffold |
| KMp88 | pFUGW-CMVp-GFP-U6p-PD1sg-E + F scaffold |
| KMp89 | pFUGW-CMVp-GFP-U6p-PD1sg-E + F G62A/A64G scaffold |
| KMp90 | pFUGW-CMVp-GFP-U6p-PD1sg-E + F U61C/A66G scaffold |
| KMp91 | pFUGW-CMVp-GFP-U6p-PD1sg-5E scaffold |
| KMp94 | pFUGW-CMVp-GFP-U6p-DNMT1sg site 4-E + F G62A/A64G |
| | scaffold |
| KMp95 | pFUGW-CMVp-GFP-U6p-DNMT1sg site 4-E + F U61C/A66G |
| | scaffold |
| KMp96 | pFUGW-CMVp-GFP-U6p-DNMT1sg site 4-5E scaffold |
| KMp99 | pFUGW-CMVp-GFP-U6p-HPRTsg-E + F G62A/A64G scaffold |
| pAWp28 | pBT264-U6p-{2xBbsI}-sgRNA scaffold-{MfeI} |
| pAWp30 | pFUGW-EFSp-Cas9-P2A-Zeo |
| pAWp63- | pFUGW-EFS-SpCas9(R661A + K1003H)-Zeo |
| clone32 | |
| pAWp9 | pFUGW-UBCp-RFP-CMVp-GFP-{BamHI + EcoRI} |
| pAWp9- | pFUGW-UBCp-RFP-CMVp-GFP-U6p-RFPsg5-ON-WT scaffold |
| R5 | |
| pJF60b | pFUGW-CMVp-AncBE4max-P2A-EGFP |
| pJHp11 | pBT264-U6p-{2xBbsI}-E + F G62A/A64G scaffold |
| pJHp12 | pBT264-U6p-{2xBbsI}-cr772 scaffold |
| pJHp13 | pBT264-U6p-{2xBbsI}-5E scaffold |
| pJHp3 | pBT264-U6p-{2xBbsI}-E + F scaffold |
| pJHp5 | pBT264-U6p-{2xBbsI}-G62A/A64G scaffold |
| pJHp6 | pBT264-U6p-{2xBbsI}-U61C/A66G scaffold |
| pKMp17 | pFUGW-hUbCp-RFP-CMVp-GFP-U6p-RFPsg5-OFF5-2-WT |
| | scaffold |
| pKMp19 | pFUGW-hUbCp-RFP-CMVp-GFP-U6p-RFPsg5-OFF5-2-cr772 |
| | scaffold |
| pKMp20 | pFUGW-hUbCp-RFP-CMVp-GFP-U6p-RFPsg5-OFF5-2-5E |
| | scaffold |
| pPZp132 | pFUGW-CMVp-GFP-U6p-FANCFsg site 6-WT scaffold |
| pPZp133 | pFUGW-CMVp-GFP-U6p-EMX1sg site 3-WT scaffold |
| pPZp138- | pFUGW-CMVp-GFP-mutH1p-dummysg3-sgRNA scaffold-U6p- |
| 3-4D | dummysg1-v1 sgRNA scaffold-mutmU6p-dummysg2-v2 sgRNA |
| | scaffold |
| pPZp156- | pFUGW-CMVp-GFP-U6p-PD1sg-WT scaffold |
| 2 | |
| pPZp257 | pFUGW-hUbCp-turboRFP-U6p-{2xEsp3I}-PE reporter b- |
| | mutmU6p-dummysg2 |
| pPZp284a | pFUGW-hUbCp-turboRFP-U6p-pegSacI-partial scaffold- |
| | {2xEsp3I}-1GTAiR13P13-PE reporter b-mutmU6p-dummysg2 |
| pPZp415 | pFUGW-EFSp-mTagBFP-U6p-EMX1sg site 3-WT scaffold |
| pPZp416 | pFUGW-EFSp-mTagBFP-U6p-CXCR4sg-WT scaffold |
| pPZp417 | pFUGW-EFSp-mTagBFP-U6p-EMX1sg site 2-WT scaffold |
| pPZp418 | pFUGW-EFSp-mTagBFP-U6p-FANCFsg site 3-WT scaffold |
| pPZp419 | pFUGW-EFSp-mTagBFP-U6p-FANCFsg site 1-WT scaffold |
| pPZp420 | pFUGW-EFSp-mTagBFP-U6p-HBGsg4-WT scaffold |
| pPZp421 | pFUGW-EFSp-mTagBFP-U6p-EMX1sg site 3-5E scaffold |
| pPZp422 | pFUGW-EFSp-mTagBFP-U6p-CXCR4sg-5E scaffold |
| pPZp423 | pFUGW-EFSp-mTagBFP-U6p-EMX1sg site 2-5E scaffold |
| pPZp424 | pFUGW-EFSp-mTagBFP-U6p-FANCFsg site 3-5E scaffold |
| pPZp425 | pFUGW-EFSp-mTagBFP-U6p-FANCFsg site 1-5E scaffold |
| pPZp426 | pFUGW-EFSp-mTagBFP-U6p-HBGsg4-5E scaffold |
| pPZp427 | pFUGW-EFSp-mTagBFP-U6p-EMX1sg site 3-SV48 scaffold |
| pPZp428 | pFUGW-EFSp-mTagBFP-U6p-CXCR4sg-SV48 scaffold |
| pPZp429 | pFUGW-EFSp-mTagBFP-U6p-EMX1sg site 2-SV48 scaffold |
| pPZp430 | pFUGW-EFSp-mTagBFP-U6p-FANCFsg site 3-SV48 scaffold |
| pPZp431 | pFUGW-EFSp-mTagBFP-U6p-FANCFsg site 1-SV48 scaffold |
| pPZp432 | pFUGW-EFSp-mTagBFP-U6p-HBGsg4-SV48 scaffold |
| pPZp433 | pFUGW-EFSp-mTagBFP-U6p-EMX1sg site 3-SV240 scaffold |
| pPZp434 | pFUGW-EFSp-mTagBFP-U6p-CXCR4sg-SV240 scaffold |
| pPZp435 | pFUGW-EFSp-mTagBFP-U6p-EMX1sg site 2-SV240 scaffold |
| pPZp436 | pFUGW-EFSp-mTagBFP-U6p-FANCFsg site 3-SV240 scaffold |
| pPZp437 | pFUGW-EFSp-mTagBFP-U6p-FANCFsg site 1-SV240 scaffold |
| pPZp438 | pFUGW-EFSp-mTagBFP-U6p-HBGsg4-SV240 scaffold |

TABLE 2
Primer sequences

| SEQ | | |
|---|---|---|
| ID | Primer | |
| NO | ID | Sequence |
| 314 | J13 | GTTGCGGATCCAAAAAAGCACCGACTCGGTGCCACTTTCT |
| | | TAAGTTGATAA |
| 315 | J14 | CGTTGCGGATCCAAAAAAGCACCGACTCGGTGCCACTCTT |
| | | TCGAGTTGATAAC |
| 316 | J15 | CTGCACTCGAGTGCAGCGAAGACCTGTTTAAGAGCTATG |
| 361 | J16 | GTTGCGGATCCAAAAAAGCACCGACTCG |
| 317 | J17 | CTGCACTCGAGTGCAGCGAAGACCTGTTTTAGAGCTAGAA |
| 318 | J18 | GTTGCGGATCCAAAAAAGCACCGACTCGGTGCCACTTTGC |
| 319 | SA82 | CGATTTCTTGGCTTTATATATCTTGTGGAA |
| 320 | SY1 | AGACCTGTTTAAGAGCTATGCTGGAAACAGCATAGCAAGT |
| | | TTAAATAAGGCTAGTCCGT |
| 321 | SY2 | AAAAAAGCACCGACTCGGTGCCACTTTTTCAAGTTGATAA |
| | | CGGACTAGCCTTATTTAAA |
| 322 | SY3 | GACCTGTTTTAGAGCTAGAAATAGCAAGTTAAAATAAGGC |
| | | TAGTCCGTTATCAA |
| 323 | SY4 | CCGACTCGGTGCCACTTTGCTGTTTCCAGCAAAGTTGATA |
| | | ACGGACTAGCCTTA |
| 324 | Z350 | GGGCACAGATAATAACCTGCAGGAGATCTAGAGGGCCTAT |
| | | TTCCC |
| 325 | Z352 | CGCGGATCCAAAAAAGGAGACGACCGGTCGTCTC-CGGTG |
| | | TTTCGTCCTTTCCACAAG |
| 326 | Z518 | CACCGAGCTCCGGAACATCCAGATCGTTTTAGAGCTAGAA |
| | | ATAGCAAGTTAAAATAAGGCTAGTCC |
| 327 | Z519 | TAACGGACTAGCCTTATTTTAACTTGCTATTTCTAGCTCT |
| | | AAAACGATCTGGATGTTCCGGAGCTC |
| 328 | Z520 | GTTATCAAGAGACGAGCGTCTCTGGCACCGAGTCGGTGCG |
| | | AGACCAGATTACCTGGATGTTCCGG |
| 329 | Z521 | AAAACCGGAACATCCAGGTAATCTGGTCTCGCACCGACTC |
| | | GGTGCCAGAGACGCTCGTCTCTTGA |

TABLE 3
Template sgRNA sequences

| SEQ ID | | |
|---|---|---|
| NO: | sgRNA name | Sequence |
| 330 | CXCR4sg | GAAGCGTGATGACAAAGAGG |
| 331 | DNMT1sg site 4 | GGAGTGAGGGAAACGGCCCC |
| 332 | dummysg1 | ATCGTTTCCGCTTAACGGCG |
| 333 | dummysg2 | AAACGGTACGACAGCGTGTG |
| 334 | EMX1sg site 2 | GTCACCTCCAATGACTAGGG |
| 335 | EMX1sg site 3 | GAGTCCGAGCAGAAGAAGAA |
| 336 | FANCFsg site 1 | GGAATCCCTTCTGCAGCACC |
| 337 | FANCFsg site 3 | GGCGGCTGCACAACCAGTGG |
| 338 | FANCFsg site 6 | GCTTGAGACCGCCAGAAGCT |
| 339 | HBGsg4 | CCTGGCTAAACTCCACCCAT |
| 340 | HPRTsg | TCGAGATGTGATGAAGGAGA |
| 341 | PD1sg | GGCCAGGATGGTTCTTAGGT |
| 342 | pegSacI | AGCTCCGGAACATCCAGATC |
| 343 | RFPsg5-ON | CACCCAGACCATGAAGATCA |
| 344 | RFPsg5-OFF5-2 | CACCCAAACCATGAAGATCA |

### Human Cell Culture

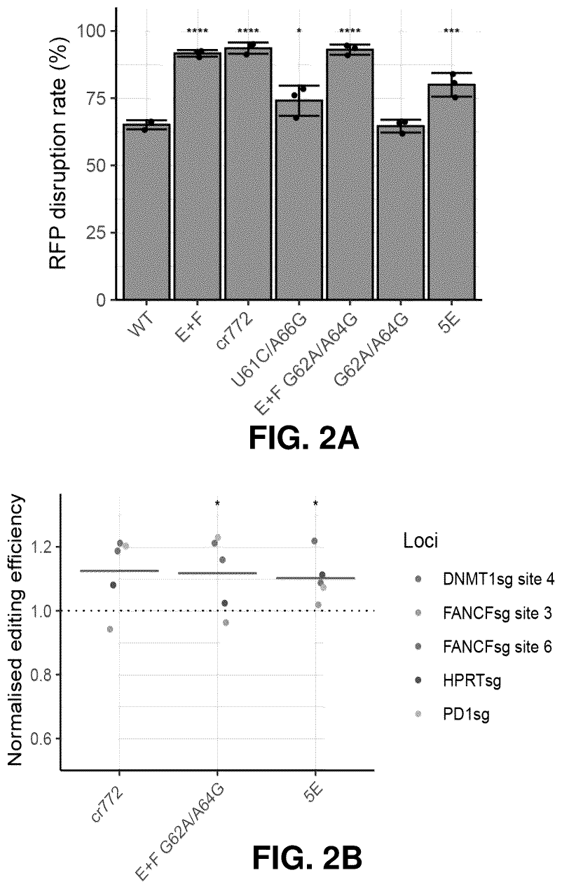

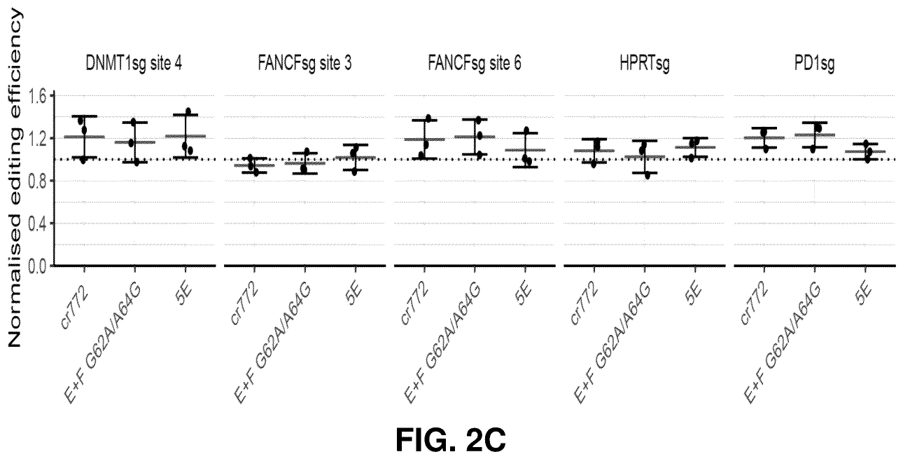

HEK293T cells were obtained from American Type Culture Collection (ATCC), and OVCAR8-ADR cells were a gift from T. Ochiya (Japanese National Cancer Center Rescarch Institute, Japan). A cell line authentication test (Genetica DNA Laboratories) was performed to confirm the identity of the OVCAR8-ADR cells. OVCAR8-ADR cells that stably express SpCas9 and AncBE4max were generated by transducing pAWp30 (Addgene, 73857) and pJF60b, followed by zeocin selection (Life Technologies) and cell sorting, respectively. Opti-SpCas9 from pAWp63-clone32 (Addgene, 131736), a high-fidelity SpCas9 that has comparable activity to wild-type (Choi, et al., Nat Methods 2019, 16, (8), 722-730), was used in the experiments shown in FIGS. 2A-2C and 3A-3C, and FIG. 7. HEK293T cells were cultured in DMEM supplemented with 10% FBS and 1× antibiotic-antimycotic (Life Technologies) at 37° ° C. with 5% CO₂. OVCAR8-ADR cells were cultured in RPMI 1640 supplemented with 10% FBS and 1× antibiotic-antimycotic at 37° C. with 5% CO₂.

### Lentiviral Transduction

For each lentivirus preparation, HEK293T cells were transfected by FuGene HD transfection reagent (Promega) according to the manufacturer's instructions in a 6-well plate, with 0.5 μg of pCMV-VSV-G, 1 μg of pCMV-dR8.2-dvpr, and 0.5 μg of the respective lentiviral vector per well. The virus-containing supernatants collected from 48 and 72 hr post-transfection were combined and filtered by 0.45 mm polyethersulfone membrane (Pall). For routine transduction, 300 μL of the filtered supernatant was applied to one well of a 12-well plate in the presence of 8 mg/ml polybrene (Sigma), with cell confluence at about 30%. For library transduction, cells were transduced by the lentiviruses at a multiplicity of infection (MOI) of <0.3 to ensure most cells were infected with just one virion. Enough cells were transduced to achieve 500-fold representation of the library size.

### Flow Cytometry and Cell Sorting

Cells for flow cytometry analysis were trypsinized and resuspended in FACS buffer (PBS with 2% FBS). BD LSR Fortessa analyser (Becton Dickinson) was used to detect the signal of TurboRFP by 561 nm yellow-green laser (610/20 nm). Data were analysed by FlowJo software (v10.5.3, Becton Dickinson). For cell sorting, samples were prepared similarly as for FACS analysis with the sorting buffer (PBS with 2% FBS and 2× antibiotic-antimycotic). BD Influx cell sorter (Becton Dickinson) equipped with a 100-mm nozzle (24 psi with a frequency of 39.2 kHz) was used. To isolate lentivirus-infected cells, fluorescent protein-positive cells were sorted using 1.0 Drop Pure mode. For cells being infected with the screening libraries, the 1%-2% cells that had the strongest fluorescent protein-positive signals were not collected to minimize the chance of acquiring cells that were infected with more than a single virion. At least 100-fold more cells than the library size were collected.

### Fluorescent Protein Disruption Assay

The on-target activity of scaffold variants was measured using a reporter system as described, in which a sgRNA spacer sequence (i.e., RFPsg5-ON) completely matched with the RFP target site. In contrast, off-target activity was measured using a reporter system in which the RFP target site contained a synonymous mutation (i.e., RFPsg5-OFF5-2). SpCas9-expressing OVCAR8-ADR cells containing the reporter system were transduced with the RFP-targeting sgRNAs containing the different scaffold variants. The fluorescent intensity was measured by flow cytometry.

### T7 Endonuclease I Assay

SpCas9-expressing OVCAR8-ADR cells were transduced with sgRNAs containing different scaffold variants, targeting endogenous loci. Genomic DNA from cells after genome editing was prepared using QuickExtract DNA extraction solution (Epicentre) or the DNeasy Blood and Tissue kit (Qiagen). The targeted loci with flanking regions were amplified by PCR and purified using PCRCleanDX (Aline Biosciences). About 300 ng of the amplicons were denatured, self-annealed, and incubated with 4 U of T7 endonuclease I (New England Biolabs) at 37° C. for 30 min. The reaction products were resolved by 2% agarose gel electrophoresis. Quantification was based on relative band intensities measured using ImageJ. Indel percentage was estimated by the formula

$$100 \times ( 1 - ( 1 - ( b + c ) / ( a + b + c ) ) 1 / 2 )$$

as previously described (Guschin, et al., Methods Mol Biol 2010, 649, 247-56), where a is the integrated intensity of the uncleaved PCR product, and b and c are the integrated intensities of each cleavage product.

### GUIDE-Seq

GUIDE-seq was performed and analysed as described. Briefly, 1 million SpCas9-expressing OVCAR8-ADR cells were transduced with sgRNA lentiviral vectors containing different scaffold variants at an MOI of ˜3 in a 6-well plate and then electroporated with 1,000 pmol dsODN at the parameters of 1,300V, 10 ms, and 3 pulses using 100-μL NEON tips (Thermo Fisher Scientific). Genomic DNAs were harvested by the DNeasy Blood and Tissue kit (Qiagen) 72 h post-electroporation and subjected to library preparation and sequencing.

### Deep Sequencing

Deep sequencing was carried out as previously described (Wong, et al., Proc Natl Acad Sci USA 2016, 113, (9), 2544-9). For validations in gene knockout and cytosine base editing settings, OVCAR8-ADR cells stably expressing SpCas9 or AncBE4max were transduced with lentiviruses of sgRNAs containing different scaffold sequences and collected on day 7 post-transduction with biological triplicates. The targeted loci were amplified from the genomic DNAs and indexed with unique barcodes by PCR. More than 0.8 million reads were obtained through NovaSeq 6000 (Illumina), evaluating editing outcomes from more than 10,000 cells for each sample. For sgRNA scaffold library screening, HEK293T cells containing the scaffold library were sorted out for pCMV_AncBE4max_P2A_GFP (Addgene, 112100) transfection and collected on day 3 post-transfection for deep sequencing. The sgRNA scaffold library-transduced OVCAR8-ADR-SpCas9 cells were collected on day 7 post-transduction. The region containing both the sgRNA scaffold variant and its targeted loci were amplified, indexed and sent for deep sequencing. CRISPresso2 (Clement, et al., Nat Biotechnol 2019, 37, (3), 224-226) was used to analyze all the deep sequencing data in NHEJ and CBE mode with default parameters. To evaluate the editing efficiency of each scaffold in the pooled library, Crispresso2 was run and surveyed the edited alleles around sgRNA from the Crispresso2 results. Alleles possessing at least 0.05% of reads and that were within the top 20 most frequently observed alleles in a sample We focused on to rule out potential defects from PCR and/or sequencing errors. Read 2s that matched with the selected alleles were then extracted and examined the sgRNA scaffold stem-loop 2 sequences at read 1s. The editing frequency of each of the sgRNA scaffold variants were counted using read Is that matched perfectly with the design sequences in the library. In the validation experiments of individual scaffolds, CRISPresso2 was run to survey their editing efficiency based on the percentage of modified reads.

### Molecular Modelling

PDB 600Y was used as the template for molecular modelling. To generate the model for sgRNA 5E, SV48, and SV240, the stem-loop 2 regions of the variants was first reconstructed using RNA composer (available on the world wide web at “//rnacomposer.cs.put.poznan.pl/”) with a pre-defined secondary structure of the intended design. Then we grafted the reconstructed stem-loop 2 to the sgRNA scaffold in the template (600Y chain B) using Rosetta (v 2019.35) RNA tools. The sgRNA variants in the reconstructed model were examined using UCSF Chimera v 1.14.

### Results

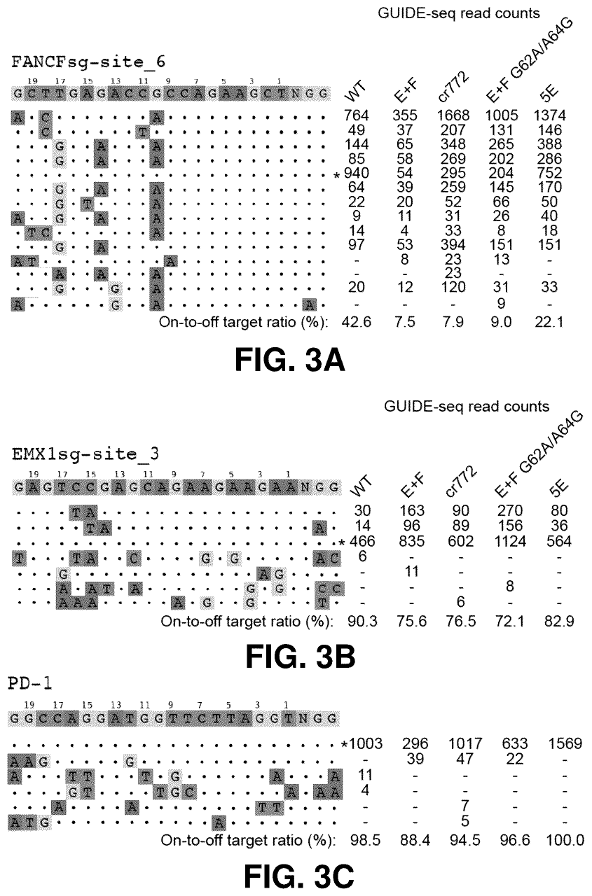

Using red fluorescent protein (RFP) disruption assay, it was confirmed that using either E+F scaffold or cr772 increased SpCas9-mediated editing to 91.7% and 93.6%, respectively, compared to 65.1% when wild-type scaffold was used (FIGS. 1, 2A). cr772 shares the same framework of E+F scaffold containing a 5-nucleotide-extended tetraloop and a A-U base-pair flip in the lower stem, but with U61C+A66G mutations. A scaffold variant with U61C+A66G mutations alone, however, did not significantly increase editing efficiency, suggesting that the E+F scaffold framework primarily contributes to the activity increase (FIG. 2A). This was further confirmed by comparing the editing efficiencies of an independent scaffold variant pair containing a replacement of the tetraloop sequence with or without the E+F framework (FIG. 2A). The tetraloop sequence AAGA (i.e., with G62A+A64G) was chosen to replace GAAA here because A64G was identified as a potential beneficial mutation in sgRNA scaffold variants (Jost, et al. Nat Biotechnol 2020, 38, (3), 355-364). At the same time, a previous study showed similar activity of an RNA/protein-interacting tetraloop containing either a AAGA or GAAA sequence (Robertson, et al., RNA 1999, 5, (9), 1167-79). an increase (˜12-13%; averaged from five loci) brought by the scaffold variants containing the E+F framework was also detected by evaluating the editing efficiency against endogenous loci using the T7 endonuclease I mismatch detection assay (FIGS. 2B-2C). Genome-wide Unbiased Identification of Double-strand breaks Enabled by sequencing (GUIDE-seq; Tsai, et al., Nat Biotechnol 2015, 33, (2), 187-97) was then applied to measure off-target activities. By assaying two endogenous loci (i.e., EMXI and FANCF) that are commonly used to benchmark off-target activities and the therapeutically relevant PD-1 locus useful for tumor eradication (Lu, et al., Nat Med 2020, 26, (5), 732-740; Rupp, et al., Sci Rep 2017, 7, (1), 737; and Su, et al., Sci Rep 2016, 6, 20070), it was found that using E+F scaffold and cr772 created new off-target sites and resulted in lower on-to-off target editing ratios than using wild-type scaffold (FIGS. 3A-3C). These results reveal that using the E+F scaffold and cr772 may come with more off-target edits.

### Example 2: Stem-Loop 2-Extended sgRNA Scaffold Variant 5E Shows Increased On-Target Activity and High Specificity

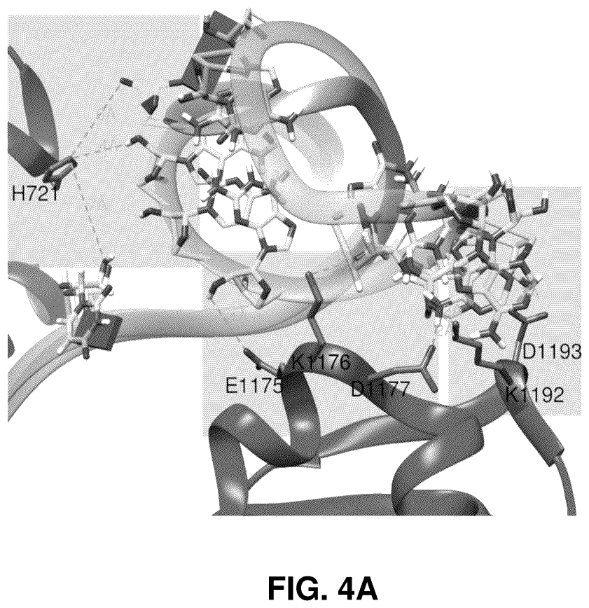

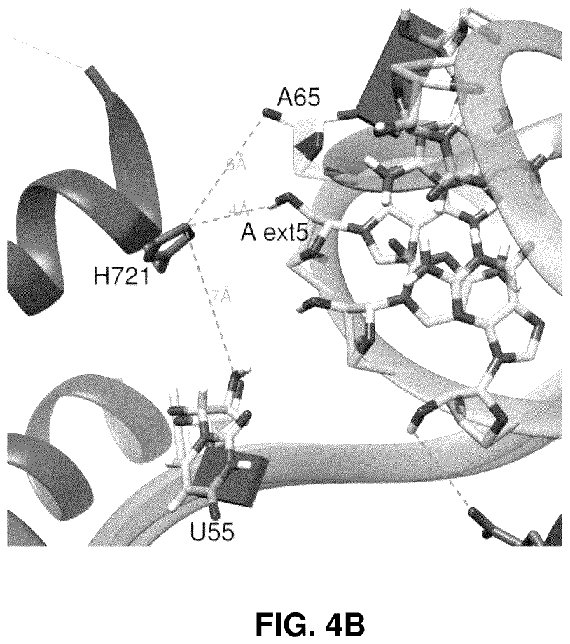

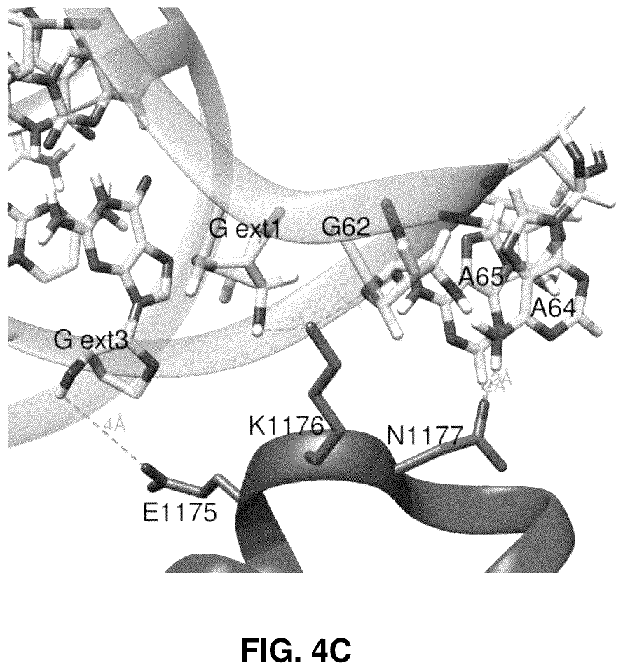

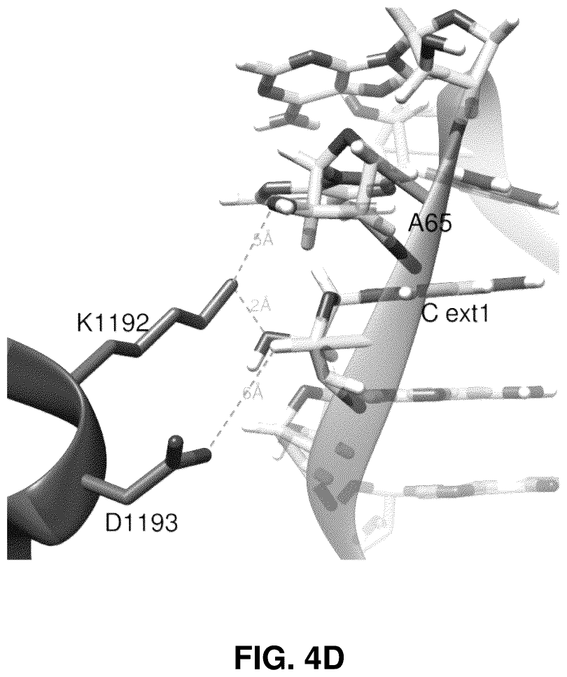

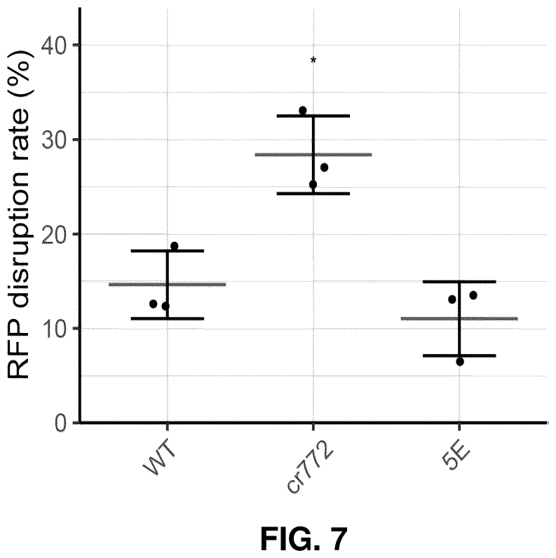

To improve SpCas9's editing activity while maintaining specificity, various regions of the sgRNA scaffold were modified. Previous studies have shown that the upper stem-loop 2 of the scaffold is positioned close to the SpCas9 (Nishimasu, et al., Cell 2014, 156, (5), 935-49) and is highly tolerant to mutations. Whether extending the upper stem-loop 2 of the scaffold could increase editing activity was investigated. A 5-nucleotide-extension was previously added to the upper stem-loop 2 in the E+F scaffold and this scaffold was shown to increase SpCas9's on-target activity (Grevet, et al., Science 2018, 361, (6399), 285-290). The scaffold variant 5E that carries only the 5-nucleotide-extension at the upper stem-loop 2 but not the other modifications present in the E+F scaffold (FIG. 2A) was therefore created. Intriguingly, it was found that while the 5E scaffold augmented the editing activity of SpCas9 from 65.1% to 80.0% according to RFP disruption (FIG. 2A) and increased 10.4% editing efficiency at five endogenous loci on average (FIGS. 2C, 3A-3C) compared to using wild-type scaffold. Using 5E scaffold resulted in a much higher on-to-off targeting ratio than when using the scaffolds with the E+F framework and did not generate new off-target sites other than those being detected for wild-type scaffold using these three sgRNAs (FIGS. 3A-3C). No off-target edits were detected when the 5E scaffold was used with a protospacer sequence targeting the PD-1 locus (FIGS. 3A-3C). 5E and wild-type scaffolds also showed greater ability to discriminate target sequences with a single-base mismatch than cr772 (FIG. 7), while the 5E scaffold generated more edits than the wild-type scaffold when used with the same protospacer sequence that targets the corresponding site without mismatch (FIG. 2A). Structurally, molecular modelling revealed that the 5-nucleotide-extension at the upper stem-loop 2 could strengthen the scaffold's interaction with SpCas9 via the His721 residue and create new interactions with two regions (E1175-N1177 and K1192 and D1193) of the PI domain of SpCas9 (FIGS. 4A-4D). These interactions may stabilize the SpCas9-sgRNA complex formation to improve editing activity. Collectively, the results show that using scaffold variant 5E could improve on-target editing while minimizing off-target editing.

### Example 3: Activity Profiling of Stem-Loop 2-Engineered sgRNA Scaffolds Identifies Variants that Increase the Editing Activity of SpCas9 Genome Editors

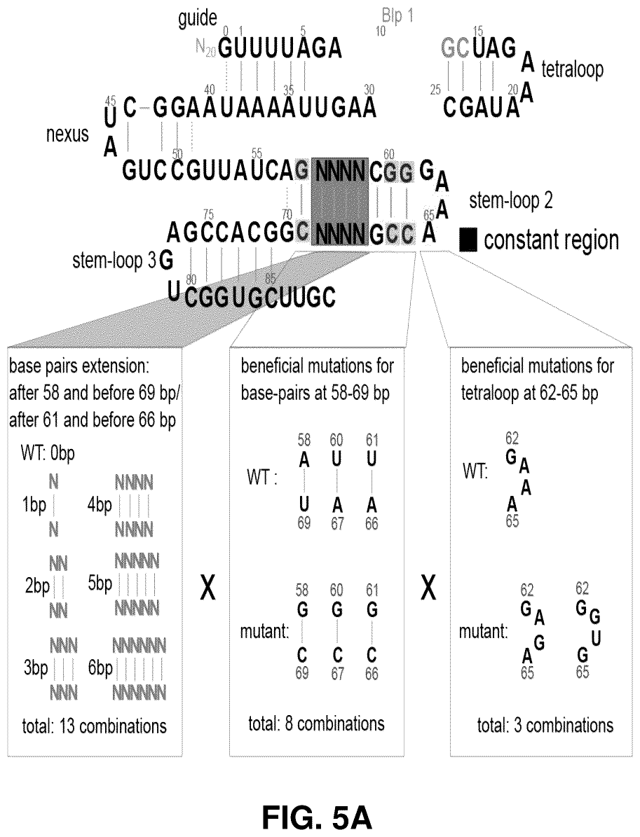

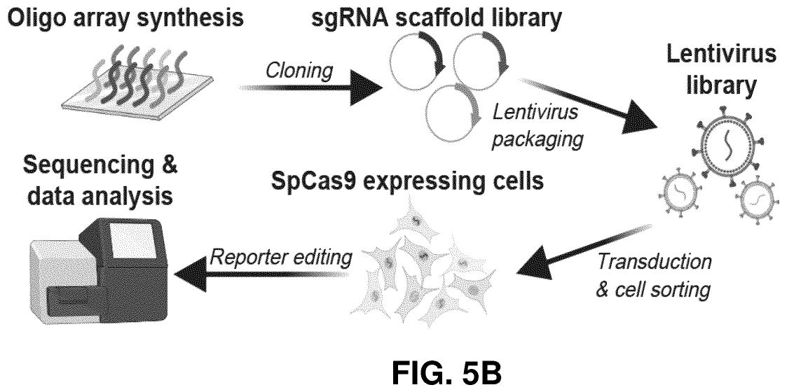

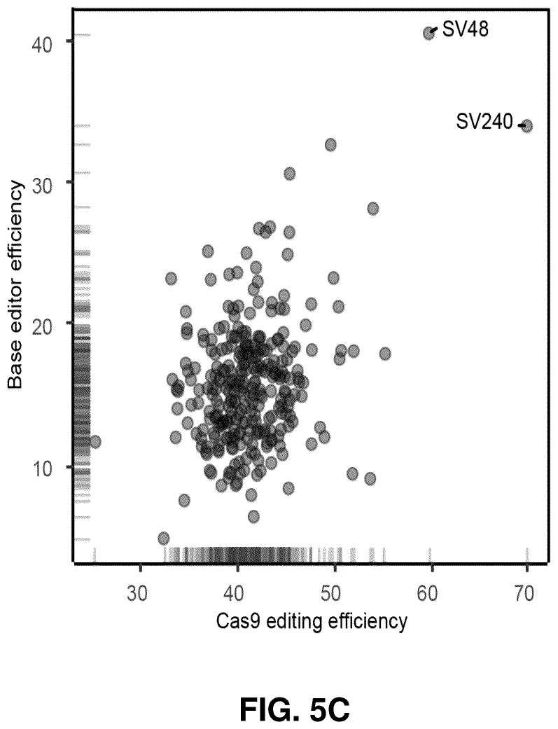

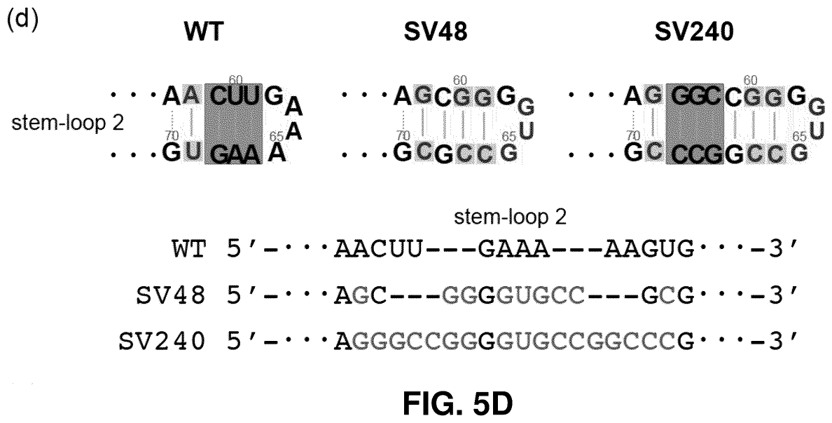

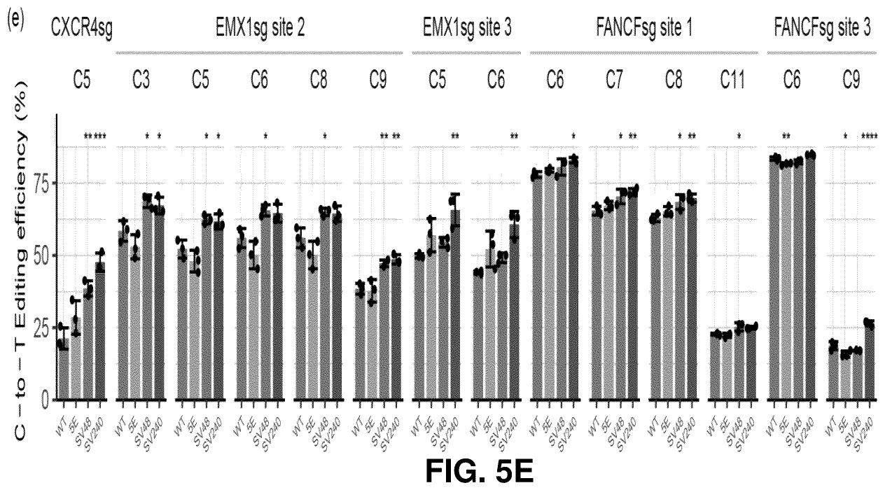

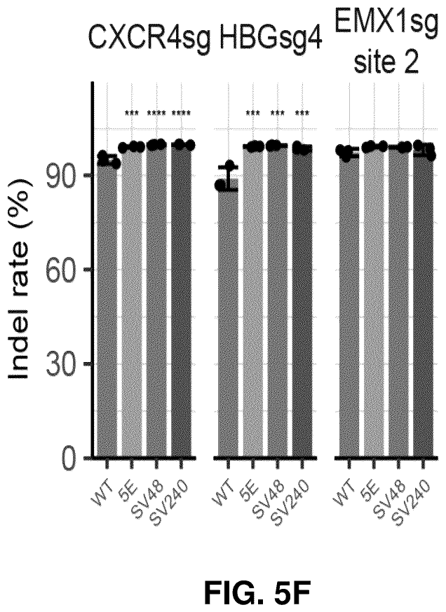

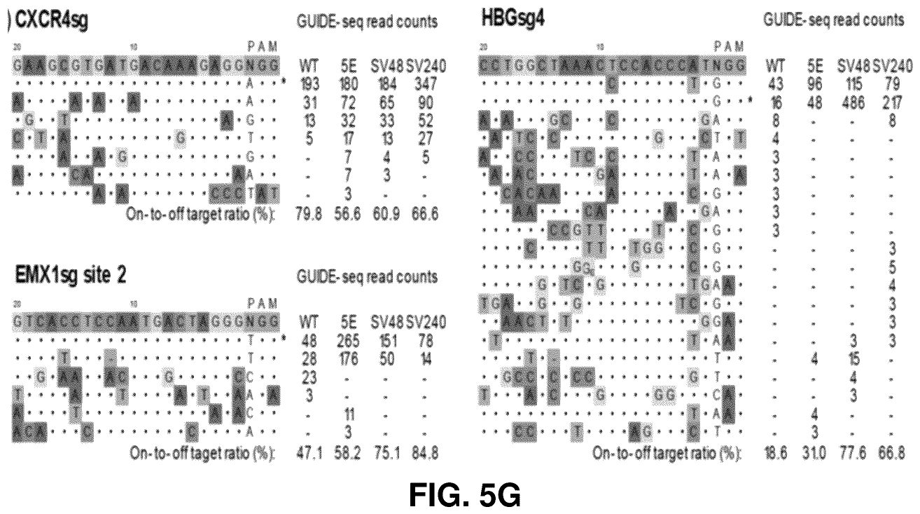

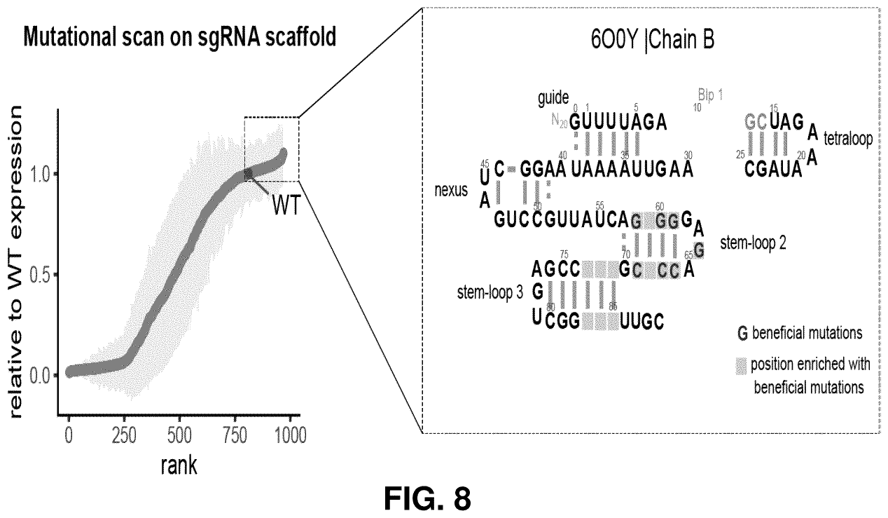

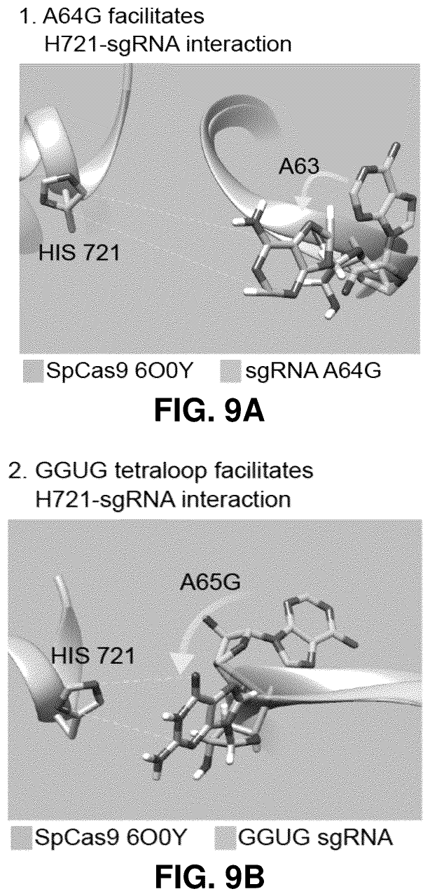

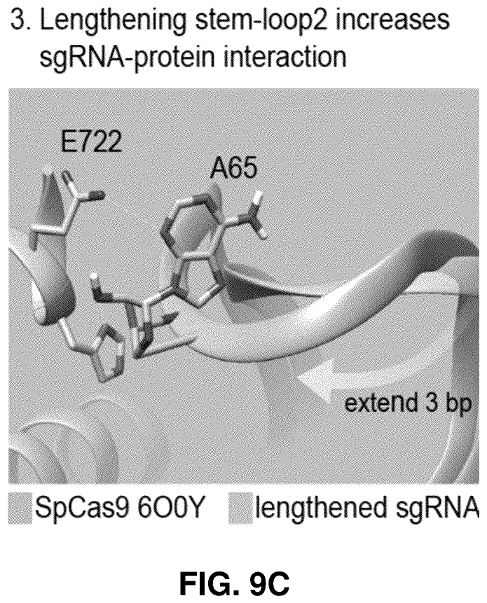

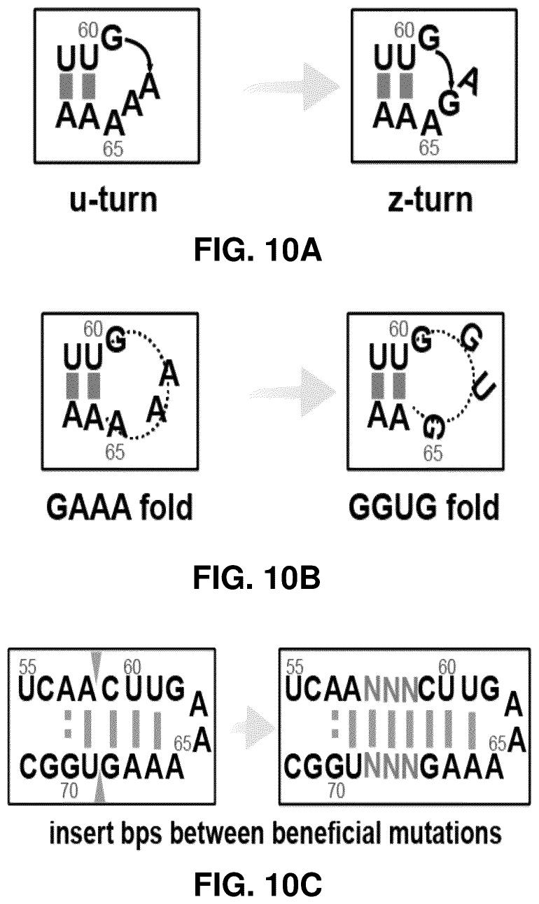

In parallel to testing scaffold variant 5E, the functional impact of introducing other modifications to the upper stem-loop 2 region of the sgRNA scaffold on modulating the activity of SpCas9 editors was also explored. Pooled screens were performed with a library of 312 scaffolds containing:
1. alternative upper stem-loop 2 sequences;
2. different lengths (1- to 6-nucleotide) of extension introduced to the upper stem-loop 2;
3. known beneficial base-pair mutations; and
4. the combinations of (1, 2 and 3) above (FIGS. 5A, 8, 9A-9C, 10A-10C; Table 4).

It was realized that some of the above modifications would strengthen the scaffold's interaction with SpCas9. The stem-loop 2 extension was designed using the RNAdesigner webserver (webpage rnasoft.ca/cgi-) and the recommended stable sequences were selected based on minimum free energy calculated by Vienna fold at temperature of 37 degrees Celsius and 50% GC content at stem regions. The library of scaffold variant-bearing sgRNAs tandemly linked to a sgRNA-targeted reporter sequence was delivered into human cells and expressed SpCas9 or its derived base editor AncBE4max to initiate editing (FIG. 5B). The editing efficiency of each sgRNA-bearing scaffold variants was quantified by Nova-seq. A base editor was used in addition to a nuclease in these screens because we sought to isolate variants that could act by strengthening the SpCas9-sgRNA scaffold interaction for broader applicability but not those affecting the nuclease's activity. The screens identified SV48 and SV240 as the best-performing scaffold variants (FIGS. 5C, 5D; Table 4). Individual validation experiments were performed and it was confirmed that both SV48 and SV240 increased SpCas9's editing, compared to using the wild-type scaffold (FIGS. 5E, 5F, 5G).

TABLE 4
sgRNA stem-loop 2 hairpin Variant
Sequences and stability data

| SEQ | Scaf- | | | CBE |
|---|---|---|---|---|
| ID | fold | | Cas | (AncBE4- |
| NO: | name | RNA Sequence | (SpCas9) | max) |
| 1 | SV1 | ACUUGGAAACAAGU | 49.5894 | 32.7056 |
| 2 | SV2 | ACUUCGAGAGAAGU | NA | NA |
| 3 | SV3 | ACUUGGGUGCAAGU | 32.3309 | 4.84055 |
| 4 | SV4 | ACUGCGAAAGCAGU | 44.6137 | 10.7882 |
| 5 | SV5 | ACUGGGAGACCAGU | 42.1766 | 26.7763 |
| 6 | SV6 | ACUGCGGUGGCAGU | 40.5085 | 17.7288 |
| 7 | SV7 | ACGUGGAAACACGU | 40.5477 | 15.5519 |
| 8 | SV8 | ACGUGGAGACACGU | 37.6624 | 13.4609 |
| 9 | SV9 | ACGUCGGUGGACGU | 37.6613 | 17.8771 |
| 10 | SV10 | ACGGGGAAACCCGU | 39.9358 | 23.6642 |
| 11 | SV11 | ACGGGGAGACCCGU | 43.3286 | 26.8854 |
| 12 | SV12 | ACGGCGGUGGCCGU | 38.0046 | 10.9862 |
| 13 | SV13 | GCUUCGAAAGAAGC | 39.6885 | 19.0912 |
| 14 | SV14 | GCUUGGAGACAAGC | 36.7852 | 11.2893 |
| 15 | SV15 | GCUUCGGUGGAAGC | 39.8686 | 15.6533 |
| 16 | SV16 | GCUGCGAAAGCAGC | 39.3419 | 14.6216 |
| 17 | SV17 | GCUGCGAGAGCAGC | 40.1531 | 13.6453 |
| 18 | SV18 | GCUGCGGUGGCAGC | 37.8424 | 16.9359 |
| 19 | SV19 | GCGUCGAAAGACGC | 34.4638 | 7.52458 |
| 20 | SV20 | GCGUGGAGACACGC | 36.4145 | 19.2414 |
| 21 | SV21 | GCGUGGGUGCACGC | 39.3501 | 18.4661 |
| 22 | SV22 | GCGGGGAAACCCGC | 37.7535 | 13.3676 |
| 23 | SV23 | GCGGGGAGACCCGC | 39.108 | 9.47179 |
| 24 | SV24 | GCGGCGGUGGCCGC | 38.4486 | 12.9953 |
| 25 | SV25 | ACUUGAAAAAGU | NA | NA |
| 26 | SV26 | ACUUGAGAAAGU | NA | NA |
| 27 | SV27 | ACUUGGUGAAGU | NA | NA |
| 28 | SV28 | ACUGGAAACAGU | NA | NA |
| 29 | SV29 | ACUGGAGACAGU | NA | NA |
| 30 | SV30 | ACUGGGUGCAGU | NA | NA |
| 31 | SV31 | ACGUGAAAACGU | NA | NA |
| 32 | SV32 | ACGUGAGAACGU | NA | NA |
| 33 | SV33 | ACGUGGUGACGU | NA | NA |
| 34 | SV34 | ACGGGAAACCGU | NA | NA |
| 35 | SV35 | ACGGGAGACCGU | NA | NA |
| 36 | SV36 | ACGGGGUGCCGU | NA | NA |
| 37 | SV37 | GCUUGAAAAAGC | NA | NA |
| 38 | SV38 | GCUUGAGAAAGC | NA | NA |
| 39 | SV39 | GCUUGGUGAAGC | NA | NA |
| 40 | SV40 | GCUGGAAACAGC | NA | NA |
| 41 | SV41 | GCUGGAGACAGC | NA | NA |
| 42 | SV42 | GCUGGGUGCAGC | NA | NA |
| 43 | SV43 | GCGUGAAAACGC | 40.3708 | 13.9277 |
| 44 | SV44 | GCGUGAGAACGC | NA | NA |
| 45 | SV45 | GCGUGGUGACGC | 42.2185 | 18.9082 |
| 46 | SV46 | GCGGGAAACCGC | 39.8585 | 19.089 |
| 47 | SV47 | GCGGGAGACCGC | 41.4769 | 13.0716 |
| 48 | SV48 | GCGGGGUGCCGC | 59.7332 | 40.5882 |
| 49 | SV49 | ACUUGCGAAAGCAAGU | 40.399 | 16.431 |
| 50 | SV50 | ACUUGGGAGACCAAGU | 44.6077 | 17.3638 |
| 51 | SV51 | ACUUCCGGUGGGAAGU | 40.5989 | 18.1672 |
| 52 | SV52 | ACUGGCGAAAGCCAGU | 44.7757 | 22.0291 |
| 53 | SV53 | ACUGGGGAGACCCAGU | 38.5695 | 19.7962 |
| 54 | SV54 | ACUGGUGGUGACCAGU | 41.6867 | 15.829 |
| 55 | SV55 | ACGUGGGAAACCACGU | 42.0179 | 12.4828 |
| 56 | SV56 | ACGUAGGAGACUACGU | 39.6507 | 20.5658 |
| 57 | SV57 | ACGUGGGGUGCCACGU | 53.9726 | 28.1939 |
| 58 | SV58 | ACGGGGGAAACCCCGU | 43.49 | 13.4536 |
| 59 | SV59 | ACGGCGGAGACGCCGU | 38.8042 | 15.4815 |
| 60 | SV60 | ACGGGCGGUGGCCCGU | 38.4465 | 16.7264 |
| 61 | SV61 | GCUUCCGAAAGGAAGC | NA | NA |
| 62 | SV62 | GCUUAGGAGACUAAGC | 36.7121 | 10.9816 |
| 63 | SV63 | GCUUGGGGUGCCAAGC | 33.6685 | 15.3781 |
| 64 | SV64 | GCUGACGAAAGUCAGC | 33.8795 | 15.5914 |
| 65 | SV65 | GCUGAGGAGACUCAGC | 44.2583 | 14.0868 |
| 66 | SV66 | GCUGUCGGUGGACAGC | 38.028 | 12.7452 |
| 67 | SV67 | GCGUACGAAAGUACGC | 37.2811 | 18.1429 |
| 68 | SV68 | GCGUCCGAGAGGACGC | 39.262 | 14.5462 |
| 69 | SV69 | GCGUUCGGUGGAACGC | 34.7264 | 19.3491 |
| 70 | SV70 | GCGGGGGAAACCCCGC | 38.0688 | 19.6849 |
| 71 | SV71 | GCGGGGGAGACCCCGC | 39.1187 | 15.7914 |
| 72 | SV72 | GCGGGGGGUGCCCCGC | 37.2736 | 18.8833 |
| 73 | SV73 | ACUUCUCGAAAGAGAAGU | 44.8376 | 15.2293 |
| 74 | SV74 | ACUUAUGGAGACAUAAGU | 42.8679 | 26.5347 |
| 75 | SV75 | ACUUGCUGGUGAGCAAGU | 41.7708 | 13.5491 |
| 76 | SV76 | ACUGCGCGAAAGCGCAGU | 39.8574 | 13.2687 |
| 77 | SV77 | ACUGCCCGAGAGGGCAGU | 51.8484 | 9.40555 |
| 78 | SV78 | ACUGCGCGGUGGCGCAGU | 44.963 | 13.6313 |
| 79 | SV79 | ACGUGGGGAAACCCACGU | 43.5188 | 11.7749 |
| 80 | SV80 | ACGUCGCGAGAGCGACGU | 44.1925 | 12.0581 |
| 81 | SV81 | ACGUAGCGGUGGCUACGU | 42.7994 | 12.4901 |
| 82 | SV82 | ACGGAGGGAAACCUCCGU | 43.9198 | 16.4668 |
| 83 | SV83 | ACGGAUGGAGACAUCCGU | 43.12 | 17.1174 |
| 84 | SV84 | ACGGCGCGGUGGCGCCGU | 43.3895 | 12.4181 |
| 85 | SV85 | GCUUCCAGAAAUGGAAGC | 42.3492 | 9.61448 |
| 86 | SV86 | GCUUGGGGAGACCCAAGC | 40.2289 | 18.0065 |
| 87 | SV87 | GCUUCCCGGUGGGGAAGC | 41.1228 | 17.7589 |
| 88 | SV88 | GCUGGGAGAAAUCCCAGC | 38.4742 | 13.092 |
| 89 | SV89 | GCUGGGGGAGACCCCAGC | 44.531 | 14.1989 |
| 90 | SV90 | GCUGGCCGGUGGGCCAGC | 40.2191 | 9.43101 |
| 91 | SV91 | GCGUUCCGAAAGGAACGC | 34.8467 | 16.6648 |
| 92 | SV92 | GCGUCUGGAGACAGACGC | 39.6189 | 12.5266 |
| 93 | SV93 | GCGUUCGGGUGCGAACGC | 38.8611 | 11.9594 |
| 94 | SV94 | GCGGCCGGAAACGGCCGC | 40.543 | 13.0221 |
| 95 | SV95 | GCGGAGGGAGACCUCCGC | 42.2539 | 15.3505 |
| 96 | SV96 | GCGGGCCGGUGGGCCCGC | 37.996 | 11.1116 |
| 97 | SV97 | ACUUCCCCGAAAGGGGAAGU | 51.9734 | 18.084 |
| 98 | SV98 | ACUUCCCCGAGAGGGGAAGU | 48.953 | 12.0051 |
| 99 | SV99 | ACUUGCCGGGUGCGGCAAGU | 44.9113 | 15.1196 |
| 100 | SV100 | ACUGCCACGAAAGUGGCAGU | 38.934 | 13.0384 |
| 101 | SV101 | ACUGGACGGAGACGUCCAGU | 42.3766 | 16.9902 |
| 102 | SV102 | ACUGCAGCGGUGGCUGCAGU | 42.9998 | 11.3545 |
| 103 | SV103 | ACGUCUGGGAAACCAGACGU | 45.8705 | 14.9822 |
| 104 | SV104 | ACGUCCGGGAGACCGGACGU | 43.599 | 16.2878 |
| 105 | SV105 | ACGUGGGAGGUGUCCCACGU | 40.1523 | 10.2151 |
| 106 | SV106 | ACGGGCUGGAAACAGCCCGU | 39.7952 | 8.60095 |
| 107 | SV107 | ACGGGGGGGAGACCCCCCGU | 44.1759 | 18.899 |
| 108 | SV108 | ACGGCUCGGGUGCGAGCCGU | 39.0422 | 14.2124 |
| 109 | SV109 | GCUUCCACGAAAGUGGAAGC | NA | NA |
| 110 | SV110 | GCUUACUCGAGAGAGUAAGC | 46.344 | 15.9114 |
| 111 | SV111 | GCUUUCCCGGUGGGGAAAGC | 42.1303 | 19.0994 |
| 112 | SV112 | GCUGACGCGAAAGCGUCAGC | NA | NA |
| 113 | SV113 | GCUGACGGGAGACCGUCAGC | 41.3786 | 13.8789 |
| 114 | SV114 | GCUGAUCCGGUGGGAUCAGC | 42.1519 | 14.2487 |
| 115 | SV115 | GCGUGCACGAAAGUGCACGC | NA | NA |
| 116 | SV116 | GCGUCUGCGAGAGCAGACGC | 40.9886 | 11.8927 |
| 117 | SV117 | GCGUGCGGGGUGCCGCACGC | 38.3631 | 12.8487 |
| 118 | SV118 | GCGGUUCCGAAAGGAACCGC | 40.2703 | 11.5098 |
| 119 | SV119 | GCGGCCAUGAGAAUGGCCGC | 38.0926 | 14.1965 |
| 120 | SV120 | GCGGUGGCGGUGGCCACCGC | 41.3951 | 10.9526 |
| 121 | SV121 | ACUUAAAGCGAAAGCUUUAAGU | 50.6754 | 18.1121 |
| 122 | SV122 | ACUUCGUCCGAGAGGACGAAGU | 44.4578 | 12.5287 |
| 123 | SV123 | ACUUAGAGCGGUGGCUCUAAGU | 43.4127 | 15.2623 |
| 124 | SV124 | ACUGCGCUCGAAAGAGCGCAGU | 55.2198 | 17.9006 |
| 125 | SV125 | ACUGGGUUGGAGACAACCCAGU | 46.7622 | 15.8637 |
| 126 | SV126 | ACUGCUGCGGGUGCGCAGCAGU | 41.2301 | 12.4429 |
| 127 | SV127 | ACGUCUCCGGAAACGGAGACGU | 39.4873 | 11.1983 |
| 128 | SV128 | ACGUGGGCAGAGAUGCCCACGU | 42.4882 | 16.7549 |
| 129 | SV129 | ACGUAGGGGGGUGCCCCUACGU | 41.4748 | 17.6034 |
| 130 | SV130 | ACGGAUCGCGAAAGCGAUCCGU | 36.9048 | 25.174 |
| 131 | SV131 | ACGGCACCCGAGAGGGUGCCGU | 45.66 | 13.0942 |
| 132 | SV132 | ACGGCGCCGGGUGCGGCGCCGU | 43.2409 | 13.3254 |
| 133 | SV133 | GCUUCACGCGAAAGCGUGAAGC | 53.6806 | 9.06075 |
| 134 | SV134 | GCUUACGGGGAGACCCGUAAGC | 43.4749 | 20.954 |
| 135 | SV135 | GCUUGAGGGGGUGCCCUCAAGC | 37.1177 | 13.3015 |
| 136 | SV136 | GCUGCGAGCGAAAGCUCGCAGC | NA | NA |
| 137 | SV137 | GCUGCGUACGAGAGUACGCAGC | 47.5814 | 21.4333 |
| 138 | SV138 | GCUGGCGUCGGUGGACGCCAGC | 39.5172 | 11.7058 |
| 139 | SV139 | GCGUGAGGGGAAACCCUCACGC | 44.3447 | 21.1201 |
| 140 | SV140 | GCGUCUUCCGAGAGGAAGACGC | 33.2296 | 16.069 |
| 141 | SV141 | GCGUCCCGGGGUGCCGGGACGC | 37.6234 | 12.0039 |
| 142 | SV142 | GCGGAUGGGGAAACCCAUCCGC | 43.7316 | 17.3696 |
| 143 | SV143 | GCGGAGGCCGAGAGGCCUCCGC | NA | NA |
| 144 | SV144 | GCGGGCGUCGGUGGACGCCCGC | 38.3023 | 8.56554 |
| 145 | SV145 | ACUUGCCCUCGAAAGAGGGCAA | 48.4996 | 12.6768 |
| | | GU | | |
| 146 | SV146 | ACUUCAGGACGAGAGUCCUGAA | 46.1747 | 16.6985 |
| | | GU | | |
| 147 | SV147 | ACUUUCCGGGGGUGCCCGGAAA | 42.026 | 17.007 |
| | | GU | | |
| 148 | SV148 | ACUGGAAUGGGAAACCAUUCCA | 50.5118 | 17.5336 |
| | | GU | | |
| 149 | SV149 | ACUGUACCGGGAGACCGGUACA | 44.1092 | 18.884 |
| | | GU | | |
| 150 | SV150 | ACUGCCUCCUGGUGAGGAGGCA | 45.3221 | 26.508 |
| | | GU | | |
| 151 | SV151 | ACGUGAGGCAGAAAUGCCUCAC | 45.1886 | 14.1661 |
| | | GU | | |
| 152 | SV152 | ACGUCUAGGGGAGACCCUAGAC | 50.4046 | 21.2538 |
| | | GU | | |
| 153 | SV153 | ACGUUCCGAGGGUGCUCGGAAC | 41.518 | 15.4363 |
| | | GU | | |
| 154 | SV154 | ACGGCGCAACGAAAGUUGCGCC | NA | NA |
| | | GU | | |
| 155 | SV155 | ACGGGCGCGCGAGAGCGCGCCC | NA | NA |
| | | GU | | |
| 156 | SV156 | ACGGACGCCAGGUGUGGCGUCC | 44.8384 | 16.2907 |
| | | GU | | |
| 157 | SV157 | GCUUCGAUCCGAAAGGAUCGAA | 45.2345 | 8.38017 |
| | | GC | | |
| 158 | SV158 | GCUUCGUCUGGAGACAGACGAA | 41.6278 | 22.4745 |
| | | GC | | |
| 159 | SV159 | GCUUGGCCUCGGUGGAGGCCAA | 42.3118 | 19.0677 |
| | | GC | | |
| 160 | SV160 | GCUGAUACCCGAAAGGGUAUCA | NA | NA |
| | | GC | | |
| 161 | SV161 | GCUGAGGAAGGAGACUUCCUCA | 41.8277 | 17.6803 |
| | | GC | | |
| 162 | SV162 | GCUGAGAGCUGGUGAGCUCUCA | 37.8037 | 14.5629 |
| | | GC | | |
| 163 | SV163 | GCGUGCACGCGAAAGCGUGCAC | NA | NA |
| | | GC | | |
| 164 | SV164 | GCGUAACUCGGAGACGAGUUAC | 41.0202 | 17.9534 |
| | | GC | | |
| 165 | SV165 | GCGUGCACGCGGUGGCGUGCAC | 41.5157 | 14.4118 |
| | | GC | | |
| 166 | SV166 | GCGGGUAGAGGAAACUCUACCC | 36.2662 | 11.3511 |
| | | GC | | |
| 167 | SV167 | GCGGCGGUCGGAGACGACCGCC | 43.456 | 11.7744 |
| | | GC | | |
| 168 | SV168 | GCGGCCCGCAGGUGUGCGGGCC | 41.9845 | 9.34056 |
| | | GC | | |
| 169 | SV169 | AGCUUGAAAAAGCU | 34.7383 | 19.6455 |
| 170 | SV170 | AGCUUGAGAAAGCU | 33.7989 | 15.2643 |
| 171 | SV171 | AGCUUGGUGAAGCU | 40.485 | 19.436 |
| 172 | SV172 | ACCUGGAAACAGGU | 41.6328 | 15.0029 |
| 173 | SV173 | AGCUGGAGACAGCU | 41.084 | 18.4555 |
| 174 | SV174 | AGCUGGGUGCAGCU | 40.8463 | 18.5321 |
| 175 | SV175 | AGCGUGAAAACGCU | 39.2339 | 16.6646 |
| 176 | SV176 | AUCGUGAGAACGAU | 34.6075 | 17.2083 |
| 177 | SV177 | AGCGUGGUGACGCU | 41.7753 | 18.5244 |
| 178 | SV178 | ACCGGGAAACCGGU | 40.7348 | 17.2507 |
| 179 | SV179 | ACCGGGAGACCGGU | 37.7021 | 16.3538 |
| 180 | SV180 | ACCGGGGUGCCGGU | 36.4102 | 12.4942 |
| 181 | SV181 | GGCUUGAAAAAGCC | 33.7457 | 14.013 |
| 182 | SV182 | GCCUUGAGAAAGGC | 37.3974 | 15.4329 |
| 183 | SV183 | GCCUUGGUGAAGGC | 39.7107 | 14.5002 |
| 184 | SV184 | GGCUGGAAACAGCC | 38.1195 | 15.9788 |
| 185 | SV185 | GCCUGGAGACAGGC | 40.1297 | 15.9452 |
| 186 | SV186 | GGCUGGGUGCAGCC | 34.6266 | 20.8922 |
| 187 | SV187 | GCCGUGAAAACGGC | 37.8756 | 13.5383 |
| 188 | SV188 | GGCGUGAGAACGCC | 38.8635 | 13.1611 |
| 189 | SV189 | GCCGUGGUGACGGC | 40.7638 | 14.1204 |
| 190 | SV190 | GGCGGGAAACCGCC | 38.1894 | 12.9759 |
| 191 | SV191 | GCCGGGAGACCGGC | 39.5812 | 15.2842 |
| 192 | SV192 | GGCGGGGUGCCGCC | 37.2246 | 16.3279 |
| 193 | SV193 | AGCCUUGAAAAAGGCU | 39.539 | 21.0942 |
| 194 | SV194 | AGGCUUGAGAAAGCCU | 40.0585 | 21.283 |
| 195 | SV195 | ACGCUUGGUGAAGCGU | 43.5253 | 16.976 |
| 196 | SV196 | AGCCUGGAAACAGGCU | 40.666 | 15.0575 |
| 197 | SV197 | AGGCUGGAGACAGCCU | 40.5708 | 17.1277 |
| 198 | SV198 | ACCCUGGGUGCAGGGU | 45.6264 | 18.0649 |
| 199 | SV199 | AGCCGUGAAAACGGCU | 40.8116 | 16.7569 |
| 200 | SV200 | AGGCGUGAGAACGCCU | 39.7469 | 14.5104 |
| 201 | SV201 | AUCCGUGGUGACGGAU | 25.2669 | 11.6701 |
| 202 | SV202 | AUCCGGGAAACCGGAU | 39.395 | 16.0226 |
| 203 | SV203 | ACGCGGGAGACCGCGU | 38.8615 | 18.2921 |
| 204 | SV204 | ACCCGGGGUGCCGGGU | 40.5702 | 15.8484 |
| 205 | SV205 | GCGCUUGAAAAAGCGC | 37.1989 | 23.1687 |
| 206 | SV206 | GGGCUUGAGAAAGCCC | 36.0053 | 15.3323 |
| 207 | SV207 | GACCUUGGUGAAGGUC | 37.7284 | 15.4118 |
| 208 | SV208 | GCCCUGGAAACAGGGC | 38.6275 | 16.6592 |
| 209 | SV209 | GCCCUGGAGACAGGGC | 42.078 | 17.8708 |
| 210 | SV210 | GGGCUGGGUGCAGCCC | 33.1035 | 23.2373 |
| 211 | SV211 | GCGCGUGAAAACGCGC | 34.807 | 12.9907 |
| 212 | SV212 | GGCCGUGAGAACGGCC | 35.5634 | 16.8754 |
| 213 | SV213 | GGGCGUGGUGACGCCC | 40.0549 | 19.734 |
| 214 | SV214 | GGCCGGGAAACCGGCC | 35.143 | 16.0594 |
| 215 | SV215 | GCCCGGGAGACCGGGC | 39.0465 | 17.0341 |
| 216 | SV216 | GCUCGGGGUGCCGAGC | 35.2006 | 14.2841 |
| 217 | SV217 | AUCCCUUGAAAAAGGGAU | 42.481 | 14.8408 |
| 218 | SV218 | ACAGCUUGAGAAAGCUGU | 39.3852 | 11.2659 |
| 219 | SV219 | ACGGCUUGGUGAAGCCGU | 41.8659 | 18.0459 |
| 220 | SV220 | AGGUCUGGAAACAGACCU | 40.8949 | 25.0434 |
| 221 | SV221 | AGGGCUGGAGACAGCCCU | 41.2461 | 20.77 |
| 222 | SV222 | AAGGCUGGGUGCAGCCUU | 37.2592 | 9.47095 |
| 223 | SV223 | ACCCCGUGAAAACGGGGU | 46.596 | 14.9061 |
| 224 | SV224 | ACCGCGUGAGAACGCGGU | 42.3755 | 18.2633 |
| 225 | SV225 | AGCGCGUGGUGACGCGCU | 39.0234 | 21.2612 |
| 226 | SV226 | AUCCCGGGAAACCGGGAU | 42.1271 | 10.3494 |
| 227 | SV227 | AGGGCGGGAGACCGCCCU | 42.6924 | 18.1338 |
| 228 | SV228 | ACCCCGGGGUGCCGGGGU | 44.5755 | 16.6218 |
| 229 | SV229 | GAGCCUUGAAAAAGGCUC | 36.4913 | 18.7869 |
| 230 | SV230 | GCGCCUUGAGAAAGGCGC | 41.749 | 10.8113 |
| 231 | SV231 | GGGACUUGGUGAAGUCCC | 36.7894 | 10.8089 |
| 232 | SV232 | GCCGCUGGAAACAGCGGC | 41.8004 | 15.9124 |
| 233 | SV233 | GCCGCUGGAGACAGCGGC | 42.0562 | 23.0238 |
| 234 | SV234 | GCCACUGGGUGCAGUGGC | 42.8345 | 16.6384 |
| 235 | SV235 | GGGGCGUGAAAACGCCCC | 41.6314 | 6.38002 |
| 236 | SV236 | GCACCGUGAGAACGGUGC | 37.0848 | 9.6205 |
| 237 | SV237 | GGGGCGUGGUGACGCCCC | 39.231 | 11.9259 |
| 238 | SV238 | GACCCGGGAAACCGGGUC | 38.888 | 11.8771 |
| 239 | SV239 | GGACCGGGAGACCGGUCC | 37.5336 | 12.28 |
| 240 | SV240 | GGGCCGGGGUGCCGGCCC | 69.8753 | 34.0293 |
| 241 | SV241 | AGAACCUUGAAAAAGGUUCU | 44.705 | 19.3725 |
| 242 | SV242 | ACAGUCUUGAGAAAGACUGU | 42.6809 | 12.3654 |
| 243 | SV243 | AAGCGCUUGGUGAAGCGCUU | 39.7101 | 10.0205 |
| 244 | SV244 | AUCACCUGGAAACAGGUGAU | 44.3625 | 16.1544 |
| 245 | SV245 | AGGGCCUGGAGACAGGCCCU | 44.5134 | 13.7234 |
| 246 | SV246 | AUACCCUGGGUGCAGGGUAU | 39.3316 | 13.3935 |
| 247 | SV247 | AGGCUCGUGAAAACGAGCCU | 41.9295 | 16.6874 |
| 248 | SV248 | ACCUACGUGAGAACGUAGGU | 45.8958 | 18.351 |
| 249 | SV249 | ACCCACGUGGUGACGUGGGU | 44.7837 | 21.078 |
| 250 | SV250 | AGAAGCGGGAAACCGCUUCU | 41.8755 | 24.0218 |
| 251 | SV251 | AGGGCCGGGAGACCGGCCCU | 41.267 | 14.8471 |
| 252 | SV252 | ACGCCCGGGGUGCCGGGCGU | 40.7334 | 13.0263 |
| 253 | SV253 | GCCUGCUUGAAAAAGCAGGC | 43.4941 | 18.5913 |
| 254 | SV254 | GGAUCCUUGAGAAAGGAUCC | 40.6533 | 9.65878 |
| 255 | SV255 | GUCGGCUUGGUGAAGCCGAC | 38.0092 | 11.4055 |
| 256 | SV256 | GCGGUCUGGAAACAGACCGC | 43.4637 | 10.1724 |
| 257 | SV257 | GCGACCUGGAGACAGGUCGC | 39.3784 | 19.0988 |
| 258 | SV258 | GCGACCUGGGUGCAGGUCGC | 39.056 | 9.12231 |
| 259 | SV259 | GGGGCCGUGAAAACGGCCCC | 35.7368 | 12.2333 |
| 260 | SV260 | GUAGGCGUGAGAACGCCUAC | 40.284 | 15.2874 |
| 261 | SV261 | GAGCCCGUGGUGACGGGCUC | 36.218 | 11.8885 |
| 262 | SV262 | GCCCCCGGGAAACCGGGGGC | 39.1081 | 23.5332 |
| 263 | SV263 | GGCGACGGGAGACCGUCGCC | 40.2238 | 12.307 |
| 264 | SV264 | GGGCACGGGGUGCCGUGCCC | 39.3088 | 13.7702 |
| 265 | SV265 | ACCGGCCUUGAAAAAGGCCGGU | 45.3565 | 30.6561 |
| 266 | SV266 | AUGGGGCUUGAGAAAGCCCCAU | 44.3245 | 16.3634 |
| 267 | SV267 | AUCCUACUUGGUGAAGUAGGAU | 44.4643 | 14.6818 |
| 268 | SV268 | AGCGGGCUGGAAACAGCCCGCU | 43.7875 | 16.5243 |
| 269 | SV269 | AGCACCCUGGAGACAGGGUGCU | 45.3505 | 14.6222 |
| 270 | SV270 | AGACCUCUGGGUGCAGAGGUCU | 38.1513 | 16.1451 |
| 271 | SV271 | ACACGUCGUGAAAACGACGUGU | 44.7008 | 18.4093 |
| 272 | SV272 | ACGGGGCGUGAGAACGCCCCGU | 41.8533 | 13.0866 |
| 273 | SV273 | AGGUGGCGUGGUGACGCCACCU | 45.3855 | 16.1346 |
| 274 | SV274 | AUCCCGCGGGAAACCGCGGGAU | 45.4011 | 13.4945 |
| 275 | SV275 | AGGGUCCGGGAGACCGGACCCU | 47.6096 | 11.5137 |
| 276 | SV276 | AGUAGGCGGGGUGCCGCCUACU | 40.3499 | 16.7219 |
| 277 | SV277 | GGACGCCUUGAAAAAGGCGUCC | 40.893 | 10.4773 |
| 278 | SV278 | GCGACGCUUGAGAAAGCGUCGC | 40.1513 | 17.6801 |
| 279 | SV279 | GUCGGCCUUGGUGAAGGCCGAC | 39.9035 | 8.74804 |
| 280 | SV280 | GUCCUCCUGGAAACAGGAGGAC | 41.5071 | 11.486 |
| 281 | SV281 | GUGCCCCUGGAGACAGGGGCAC | 41.6266 | 16.9063 |
| 282 | SV282 | GUCUGCCUGGGUGCAGGCAGAC | 39.3705 | 15.9213 |
| 283 | SV283 | GGCUACCGUGAAAACGGUAGCC | 39.3277 | 9.47273 |
| 284 | SV284 | GCGGGGCGUGAGAACGCCCCGC | 40.8025 | 18.4344 |
| 285 | SV285 | GCUCACCGUGGUGACGGUGAGC | 43.044 | 11.579 |
| 286 | SV286 | GCUAGCCGGGAAACCGGCUAGC | 45.0949 | 17.4832 |
| 287 | SV287 | GGCAGGCGGGAGACCGCCUGCC | 37.0721 | 13.8001 |
| 288 | SV288 | GGCCAGCGGGGUGCCGCUGGCC | 39.6025 | 12.1705 |
| 289 | SV289 | ACCACUGCUUGAAAAAGCAGUG | 49.8863 | 23.284 |
| | | GU | | |
| 290 | SV290 | ACUGUGGCUUGAGAAAGCCACA | 45.1487 | 24.9466 |
| | | GU | | |
| 291 | SV291 | AGCGCCGCUUGGUGAAGCGGCG | 40.8116 | 19.4371 |
| | | CU | | |
| 292 | SV292 | AGCCAGGCUGGAAACAGCCUGG | 47.0098 | 19.9196 |
| | | CU | | |
| 293 | SV293 | AGAGCCCCUGGAGACAGGGGCU | 46.2363 | 16.1629 |
| | | CU | | |
| 294 | SV294 | AGCACCCCUGGGUGCAGGGGUG | 42.9929 | 18.1392 |
| | | CU | | |
| 295 | SV295 | AACCCGGCGUGAAAACGCCGGG | NA | NA |
| | | UU | | |
| 296 | SV296 | ACGCUCCCGUGAGAACGGGAGC | 45.242 | 12.7944 |
| | | GU | | |
| 297 | SV297 | ACAAGGCCGUGGUGACGGCCUU | 47.6471 | 18.1639 |
| | | GU | | |
| 298 | SV298 | ACGGCCACGGGAAACCGUGGCC | 44.3448 | 11.3816 |
| | | GU | | |
| 299 | SV299 | AUGCGGACGGGAGACCGUCCGC | 42.021 | 12.2931 |
| | | AU | | |
| 300 | SV300 | AGACAGCCGGGGUGCCGGCUGU | 38.3438 | 18.1917 |
| | | CU | | |
| 301 | SV301 | GACAGAGCUUGAAAAAGCUCUG | 43.5076 | 21.5274 |
| | | UC | | |
| 302 | SV302 | GUCGCAGCUUGAGAAAGCUGCG | 42.3213 | 15.0403 |
| | | AC | | |
| 303 | SV303 | GUGGCCGCUUGGUGAAGCGGCC | 37.3747 | 15.0314 |
| | | AC | | |
| 304 | SV304 | GGUCCUCCUGGAAACAGGAGGA | 42.1202 | 21.5732 |
| | | CC | | |
| 305 | SV305 | GUCCCCGCUGGAGACAGCGGGG | 42.3051 | 12.2969 |
| | | AC | | |
| 306 | SV306 | GGUGGGCCUGGGUGCAGGCCCA | 40.1055 | 12.1839 |
| | | CC | | |
| 307 | SV307 | GUGGCUCCGUGAAAACGGAGCC | 40.8355 | 19.0856 |
| | | AC | | |
| 308 | SV308 | GCUCCGACGUGAGAACGUCGGA | 33.5717 | 11.985 |
| | | GC | | |
| 309 | SV309 | GUCUCCCCGUGGUGACGGGGAG | 39.0906 | 15.2552 |
| | | AC | | |
| 310 | SV310 | GCGAGCCCGGGAAACCGGGCUC | 41.3889 | 7.90354 |
| | | GC | | |
| 311 | SV311 | GGACCCCCGGGAGACCGGGGGU | 35.9166 | 14.4173 |
| | | CC | | |
| 312 | SV312 | GCCCUCCCGGGGUGCCGGGAGG | 36.8775 | 15.4625 |
| | | GC | | |

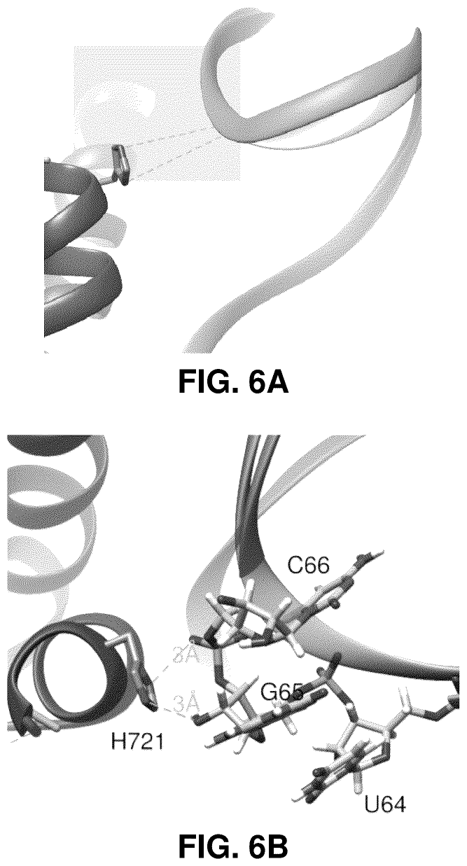

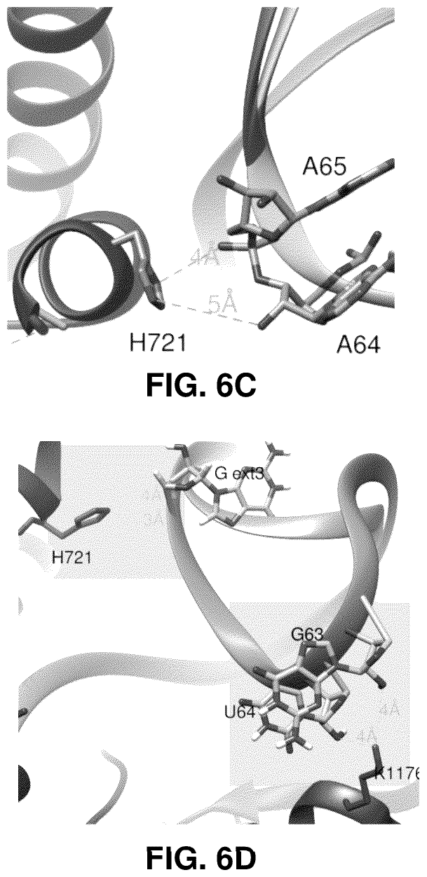

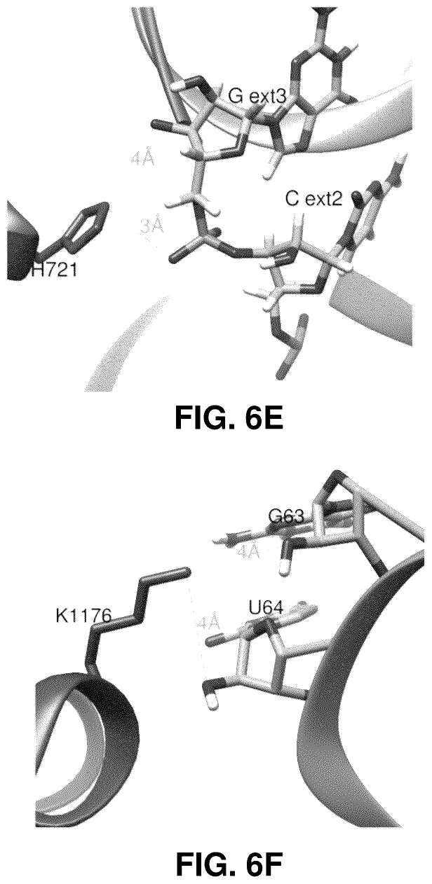

Using SV48 and SV240 generated more base edits at the five endogenous loci tested than using a wild-type scaffold (FIG. 5E). In particular, at the CXCR4 loci, SV240 increased base edits from 21.3% to 47.7% (FIG. 5E). Using SV48 and SV240 also boosted the editing activity of SpCas9 nuclease to up to 99.7% at CXCR4 and up to 99.5% at the γ-Globin Gene (HBG) promoter region (FIG. 5F). Editing using SV48 and SV240 also achieved generally high on-to-off targeting activities (i.e., >60% over all 3 loci tested) (FIG. 5G), albeit that the increased on-to-off targeting ratios observed could be locus-specific. For the therapeutically relevant HBG promoter region targeted by HBGsg4, the on-to-off target ratio increased from 18.6% to 77.6% and 66.8% when wild-type scaffold was substituted with SV48 and SV240, respectively (FIG. 5G). Both SV48 and SV240 carry a GGUG tetraloop sequence replacement at upper stem-loop 2 (FIG. 5D). Molecular modelling indicates that a GGUG tetraloop, along with the other substitutions in the stem-loop 2 regions of SV48 lead to a different loop conformation. The backbone of G65 and C66 is brought closer to His721 (at distances of 3 Å) of SpCas9 and forms two points of contacts for stronger interactions (FIGS. 6A, 6B). With wild-type scaffold, A64 and A65 at the tetraloop of stem-loop 2 interact with His721 of SpCas9 at distances of 4-5 Å (FIG. 6C). The SV240 scaffold is also modelled to strengthen existing interactions with His721 of SpCas9 and create new interactions with K1176 of the PI domain in SpCas9 (FIGS. 6D-6F). In line with the observation of the loop-extended 5E scaffold, these models indicate that strengthening the scaffold's interaction with SpCas9 via His721 and the PI domain of SpCas9 represents a viable approach to engineer Cas9 activity, and demonstrate that engineering the stem-loop 2 of the scaffold is useful for optimizing the SpCas9 genome editor's activity.

### DISCUSSION

Guide RNA engineering strategies should improve CRISPR's on-target activity while minimizing off-target edits. Intriguingly, it was found that the previously reported sgRNA scaffold variants increase off-target editing more than on-target activity. sgRNA scaffold variants that augment on-target CRISPR editing while achieving high on-to-off targeting specificity have been engineered. Although the exact mechanism on how extending the upper stem-loop 2 alone in these new scaffolds may give such an advantage remains to be understood, molecular modelling hints that it is related to the increase in the scaffold's interaction with His721 and the PI domain of SpCas9. These interactions are distant from where the extended tetraloop in the previously engineered E+F scaffold interacts with SpCas9 (Nishimasu, et al. Cell 2014, 156, (5), 935-49), suggesting that the described scaffolds modulate SpCas9's editing activity via a different mechanism. Strengthened sgRNA:SpCas9 binding via His721 and PI domain interactions with the scaffold may further favor sgRNA loading over competitor intracellular RNA binding (Mekler, et al., Nucleic Acids Res 2016, 44, (6), 2837-45), thus stabilizing Cas9-sgRNA complex formation and enhancing editing activity. At the same time, it remains to be revealed whether it may also render the neighboring RuvC domain less energetically favorable to form a reorganized loop to stabilize target DNA substrate with mismatches (Bravo, et al., Nature 2022, 603, (7900), 343-347) or act through other mechanisms to minimize off-target editing. The data presented above also revealed that the same stem-loop 2-engineered scaffolds could be useful for enhancing the activities of base editors derived from SpCas9. Some scaffolds may adopt different sgRNA design rules. Indeed, the engineering of sgRNA scaffolds is still in its infancy, particularly for those effectors, including prime editor and Cas12f (Nelson, et al., Nat Biotechnol 2022, 40, (3), 402-410; Kim, et al., Nat Biotechnol 2022, 40, (1), 94-102; and Xu, et al., Mol Cell 2021, 81, (20), 4333-4345 e4), that were shown to require more extensive modifications.

In summary, the data have uncovered an engineering route to create new stem-loop 2-modified sgRNA scaffolds for increasing the editing activity of both SpCas9 nuclease and base editor. This work demonstrates the feasibility of engineering sgRNA scaffold variants for SpCas9 to achieve both high efficiency and specificity, highlighting applications for applying high-throughput sgRNA scaffold engineering approaches to enhance the CRISPR-Cas systems for genome editing applications.

Those skilled in the art will recognize or be able to ascertain using no more than routine experimentation, many equivalents to the specific embodiments of the method and compositions described herein. Such equivalents are intended to be encompassed by the following claims.

We claim:

1. A variant single guide RNA (sgRNA) comprising substitution and/or addition of one or more nucleic acid residues that strengthens the interaction of the sgRNA with a Cas enzyme,
wherein the strengthened interaction imparts increased on-target editing and/or increased on-off target specificity relative to a wild type sgRNA that lacks the substitution and/or addition of one or more nucleic acid residues.

2. The variant sgRNA of claim 1, wherein the substitution and/or addition of one or more nucleic acid residues that strengthens the interaction of the sgRNA with a Cas enzyme comprises substitution and/or addition of one or more nucleic acid residues within the hairpin region of the stem-loop 2 of the sgRNA.

3. The variant sgRNA of claim 1, wherein the Cas enzyme is a Cas9 enzyme.

4. The variant sgRNA of claim 3, wherein the Cas9 enzyme is derived from Streptococcus pyogenes (spCas9).

5. The variant sgRNA of claim 4, wherein the substitution and/or addition of one or more nucleic acid residues strengthens the sgRNAs interaction with residue His721 and/or the PI domain of SpCas9.

6. The variant sgRNA of claim 2, comprising the nucleic acid sequence:
(SEQ ID NO: 355)
GUUUUAGAGCUAGAAAUAGCAAGUUAAAAUAAGGCUAGUCCGUUAUCA-
X-GGCACCGAGUCGGUGCU,
wherein “—X—” represents a hairpin region of stem-loop 2 comprising between 12 and 24 nucleic acid residues, inclusive.

7. The variant sgRNA of claim 2, wherein the hairpin region of stem-loop 2 comprises the nucleic acid sequence of any one of SEQ ID NOS: 1-312.

8. The variant sgRNA of claim 2, wherein the hairpin region of stem-loop 2 comprises the nucleic acid sequence GCGGGGUGCCGC (SEQ ID NO:48), or a nucleic acid sequence having at least about 74% identity to SEQ ID NO:48, or a nucleic acid sequence having at least 82%, or at least 91% sequence identity to GCGGGGUGCCGC (SEQ ID NO:48).

9. The variant sgRNA of claim 2, wherein the hairpin region of stem-loop 2 comprises the nucleic acid sequence GGGCCGGGGUGCCGGCCC (SEQ ID NO:240), or a nucleic acid sequence having at least about 75% identity to SEQ ID NO:240, or a nucleic acid sequence having at least 77%, at least 82%, at least 88%, or at least 94% sequence identity to GGGCCGGGGUGCCGGCCC (SEQ ID NO:240).

10. The variant sgRNA of claim 1, comprising a nucleic acid sequence of GUUUUAGAGCUAGAAAUAGCAAGUUAAAAUAAGGCUAGUCCGUUAUCAGCGGG GUGCCGCGGCACCGAGUCGGUGCU (SEQ ID NO:352), or a nucleic acid sequence having at least 75% identity to SEQ ID NO:352, or a nucleic acid sequence having at least 80%, at least 85%, at least 90%, or at least 95% sequence identity to SEQ ID NO:352.

11. The variant sgRNA of claim 1, comprising a nucleic acid sequence of GUUUUAGAGCUAGAAAUAGCAAGUUAAAAUAAGGCUAGUCCGUUAUCAGGGCC GGGGUGCCGGCCCGGCACCGAGUCGGUGCU (SEQ ID NO:353), or a nucleic acid sequence having at least 75% identity to SEQ ID NO:353, or a nucleic acid sequence having at least 80%, at least 85%, at least 90%, or at least 95% sequence identity to SEQ ID NO:353.

12. A ribonucleoprotein complex comprising:
(a) a Cas9 enzyme; and
(b) the variant sgRNA of claim 1,
wherein the variant sgRNA comprises a hairpin region of stem-loop 2 comprising the nucleic acid sequence of any one of SEQ ID NOs:1-312,
wherein the ribonucleoprotein complex has increased on-target editing and/or increased on-off target specificity relative to the corresponding complex between a Cas9 enzyme and wild type sgRNA.

13. The ribonucleoprotein complex of claim 12, wherein the Cas9 enzyme is derived from Streptococcus pyogenes (spCas9).

14. The ribonucleoprotein complex of claim 12, wherein the variant sgRNA comprises the nucleic acid sequence:
(SEQ ID NO: 355)
GUUUUAGAGCUAGAAAUAGCAAGUUAAAAUAAGGCUAGUCCGUUAUCA-
X-GGCACCGAGUCGGUGCU,
wherein “—X—” represents a hairpin region of stem-loop 2 comprising the nucleic acid sequence of any one of SEQ ID NOs: 1-312.

15. The ribonucleoprotein complex of claim 14 comprising the sgRNA having a nucleic acid sequence of:
GUUUUAGAGCUAGAAAUAGCAAGUUAAAAUAAGGCUAGUCCGUUAUCAGCGGG GUGCCGCGGCACCGAGUCGGUGCU (SEQ ID NO:352), or a nucleic acid sequence having at least 75% identity to SEQ ID NO:352; or
GUUUUAGAGCUAGAAAUAGCAAGUUAAAAUAAGGCUAGUCCGUUAUCAGGGCC GGGGUGCCGGCCCGGCACCGAGUCGGUGCU (SEQ ID NO:353), or a nucleic acid sequence having at least 75% identity to SEQ ID NO:353.

16. A vector encoding or expressing the variant single guide RNA (sgRNA) of claim 1,
optionally wherein the substitution and/or addition of one or more nucleic acid residues that strengthens the interaction of the sgRNA with a Cas enzyme comprises substitution and/or addition of one or more nucleic acid residues within the hairpin region of the stem-loop 2 of the sgRNA of claim 1.

17. A cell comprising
(i) the sgRNA vector of claim 16; or
(ii) a ribonucleoprotein complex, comprising:
(a) a Cas9 enzyme; and
(b) a variant sgRNA,
wherein the variant sgRNA comprises a hairpin region of stem-loop 2 comprising the nucleic acid sequence of any one of SEQ ID NOs:1-312, and
wherein the ribonucleoprotein complex has increased on-target editing and/or increased on-off target specificity relative to the corresponding complex between a Cas9 enzyme and wild type sgRNA.

18. A method for CRISPR editing of one or more target genes in a cell, the method comprising administering into and/or expressing within the cell the ribonucleoprotein complex of claim 12,
wherein the ribonucleoprotein complex is configured to target the one or more target genes.

19. The method of claim 18, wherein the administering is in vivo.

20. A kit comprising
(i) a variant single guide RNA (sgRNA), comprising substitution and/or addition of one or more nucleic acid residues that strengthens the interaction of the sgRNA with a Cas enzyme, and
wherein the strengthened interaction imparts increased on-target editing and/or increased on-off target specificity relative to a wild type sgRNA that lacks the substitution and/or addition of one or more nucleic acid residues,
optionally wherein the substitution and/or addition of one or more nucleic acid residues that strengthens the interaction of the sgRNA with a Cas enzyme comprises substitution and/or addition of one or more nucleic acid residues within the hairpin region of the stem-loop 2 of the sgRNA; and optionally
(ii) a Cas9 enzyme, or vector encoding or expressing the Cas9 enzyme; and/or
(iii) instructions for performing the method of claim 18.


SEQUENCE LISTING (364 sequences)

SEQ ID NO: 1 - RNA, 14 nt, synthetic construct
  source 1..14: mol_type = other RNA
acttggaaacaagt

SEQ ID NO: 2 - RNA, 14 nt, synthetic construct
  source 1..14: mol_type = other RNA
acttcgagagaagt

SEQ ID NO: 3 - RNA, 14 nt, synthetic construct
  source 1..14: mol_type = other RNA
acttgggtgcaagt

SEQ ID NO: 4 - RNA, 14 nt, synthetic construct
  source 1..14: mol_type = other RNA
actgcgaaagcagt

SEQ ID NO: 5 - RNA, 14 nt, synthetic construct
  source 1..14: mol_type = other RNA
actgggagaccagt

SEQ ID NO: 6 - RNA, 14 nt, synthetic construct
  source 1..14: mol_type = other RNA
actgcggtggcagt

SEQ ID NO: 7 - RNA, 14 nt, synthetic construct
  source 1..14: mol_type = other RNA
acgtggaaacacgt

SEQ ID NO: 8 - RNA, 14 nt, synthetic construct
  source 1..14: mol_type = other RNA
acgtggagacacgt

SEQ ID NO: 9 - RNA, 14 nt, synthetic construct
  source 1..14: mol_type = other RNA
acgtcggtggacgt

SEQ ID NO: 10 - RNA, 14 nt, synthetic construct
  source 1..14: mol_type = other RNA
acggggaaacccgt

SEQ ID NO: 11 - RNA, 14 nt, synthetic construct
  source 1..14: mol_type = other RNA
acggggagacccgt

SEQ ID NO: 12 - RNA, 14 nt, synthetic construct
  source 1..14: mol_type = other RNA
acggcggtggccgt

SEQ ID NO: 13 - RNA, 14 nt, synthetic construct
  source 1..14: mol_type = other RNA
gcttcgaaagaagc

SEQ ID NO: 14 - RNA, 14 nt, synthetic construct
  source 1..14: mol_type = other RNA
gcttggagacaagc

SEQ ID NO: 15 - RNA, 14 nt, synthetic construct
  source 1..14: mol_type = other RNA
gcttcggtggaagc

SEQ ID NO: 16 - RNA, 14 nt, synthetic construct
  source 1..14: mol_type = other RNA
gctgcgaaagcagc

SEQ ID NO: 17 - RNA, 14 nt, synthetic construct
  source 1..14: mol_type = other RNA
gctgcgagagcagc

SEQ ID NO: 18 - RNA, 14 nt, synthetic construct
  source 1..14: mol_type = other RNA
gctgcggtggcagc

SEQ ID NO: 19 - RNA, 14 nt, synthetic construct
  source 1..14: mol_type = other RNA
gcgtcgaaagacgc

SEQ ID NO: 20 - RNA, 14 nt, synthetic construct
  source 1..14: mol_type = other RNA
gcgtggagacacgc

SEQ ID NO: 21 - RNA, 14 nt, synthetic construct
  source 1..14: mol_type = other RNA
gcgtgggtgcacgc

SEQ ID NO: 22 - RNA, 14 nt, synthetic construct
  source 1..14: mol_type = other RNA
gcggggaaacccgc

SEQ ID NO: 23 - RNA, 14 nt, synthetic construct
  source 1..14: mol_type = other RNA
gcggggagacccgc

SEQ ID NO: 24 - RNA, 14 nt, synthetic construct
  source 1..14: mol_type = other RNA
gcggcggtggccgc

SEQ ID NO: 25 - RNA, 12 nt, synthetic construct
  source 1..12: mol_type = other RNA
acttgaaaaagt

SEQ ID NO: 26 - RNA, 12 nt, synthetic construct
  source 1..12: mol_type = other RNA
acttgagaaagt

SEQ ID NO: 27 - RNA, 12 nt, synthetic construct
  source 1..12: mol_type = other RNA
acttggtgaagt

SEQ ID NO: 28 - RNA, 12 nt, synthetic construct
  source 1..12: mol_type = other RNA
actggaaacagt

SEQ ID NO: 29 - RNA, 12 nt, synthetic construct
  source 1..12: mol_type = other RNA
actggagacagt

SEQ ID NO: 30 - RNA, 12 nt, synthetic construct
  source 1..12: mol_type = other RNA
actgggtgcagt

SEQ ID NO: 31 - RNA, 12 nt, synthetic construct
  source 1..12: mol_type = other RNA
acgtgaaaacgt

SEQ ID NO: 32 - RNA, 12 nt, synthetic construct
  source 1..12: mol_type = other RNA
acgtgagaacgt

SEQ ID NO: 33 - RNA, 12 nt, synthetic construct
  source 1..12: mol_type = other RNA
acgtggtgacgt

SEQ ID NO: 34 - RNA, 12 nt, synthetic construct
  source 1..12: mol_type = other RNA
acgggaaaccgt

SEQ ID NO: 35 - RNA, 12 nt, synthetic construct
  source 1..12: mol_type = other RNA
acgggagaccgt

SEQ ID NO: 36 - RNA, 12 nt, synthetic construct
  source 1..12: mol_type = other RNA
acggggtgccgt

SEQ ID NO: 37 - RNA, 12 nt, synthetic construct
  source 1..12: mol_type = other RNA
gcttgaaaaagc

SEQ ID NO: 38 - RNA, 12 nt, synthetic construct
  source 1..12: mol_type = other RNA
gcttgagaaagc

SEQ ID NO: 39 - RNA, 12 nt, synthetic construct
  source 1..12: mol_type = other RNA
gcttggtgaagc

SEQ ID NO: 40 - RNA, 12 nt, synthetic construct
  source 1..12: mol_type = other RNA
gctggaaacagc

SEQ ID NO: 41 - RNA, 12 nt, synthetic construct
  source 1..12: mol_type = other RNA
gctggagacagc

SEQ ID NO: 42 - RNA, 12 nt, synthetic construct
  source 1..12: mol_type = other RNA
gctgggtgcagc

SEQ ID NO: 43 - RNA, 12 nt, synthetic construct
  source 1..12: mol_type = other RNA
gcgtgaaaacgc

SEQ ID NO: 44 - RNA, 12 nt, synthetic construct
  source 1..12: mol_type = other RNA
gcgtgagaacgc

SEQ ID NO: 45 - RNA, 12 nt, synthetic construct
  source 1..12: mol_type = other RNA
gcgtggtgacgc

SEQ ID NO: 46 - RNA, 12 nt, synthetic construct
  source 1..12: mol_type = other RNA
gcgggaaaccgc

SEQ ID NO: 47 - RNA, 12 nt, synthetic construct
  source 1..12: mol_type = other RNA
gcgggagaccgc

SEQ ID NO: 48 - RNA, 12 nt, synthetic construct
  source 1..12: mol_type = other RNA
gcggggtgccgc

SEQ ID NO: 49 - RNA, 16 nt, synthetic construct
  source 1..16: mol_type = other RNA
acttgcgaaagcaagt

SEQ ID NO: 50 - RNA, 16 nt, synthetic construct
  source 1..16: mol_type = other RNA
acttgggagaccaagt

SEQ ID NO: 51 - RNA, 16 nt, synthetic construct
  source 1..16: mol_type = other RNA
acttccggtgggaagt

SEQ ID NO: 52 - RNA, 16 nt, synthetic construct
  source 1..16: mol_type = other RNA
actggcgaaagccagt

SEQ ID NO: 53 - RNA, 16 nt, synthetic construct
  source 1..16: mol_type = other RNA
actggggagacccagt

SEQ ID NO: 54 - RNA, 16 nt, synthetic construct
  source 1..16: mol_type = other RNA
actggtggtgaccagt

SEQ ID NO: 55 - RNA, 16 nt, synthetic construct
  source 1..16: mol_type = other RNA
acgtgggaaaccacgt

SEQ ID NO: 56 - RNA, 16 nt, synthetic construct
  source 1..16: mol_type = other RNA
acgtaggagactacgt

SEQ ID NO: 57 - RNA, 16 nt, synthetic construct
  source 1..16: mol_type = other RNA
acgtggggtgccacgt

SEQ ID NO: 58 - RNA, 16 nt, synthetic construct
  source 1..16: mol_type = other RNA
acgggggaaaccccgt

SEQ ID NO: 59 - RNA, 16 nt, synthetic construct
  source 1..16: mol_type = other RNA
acggcggagacgccgt

SEQ ID NO: 60 - RNA, 16 nt, synthetic construct
  source 1..16: mol_type = other RNA
acgggcggtggcccgt

SEQ ID NO: 61 - RNA, 16 nt, synthetic construct
  source 1..16: mol_type = other RNA
gcttccgaaaggaagc

SEQ ID NO: 62 - RNA, 16 nt, synthetic construct
  source 1..16: mol_type = other RNA
gcttaggagactaagc

SEQ ID NO: 63 - RNA, 16 nt, synthetic construct
  source 1..16: mol_type = other RNA
gcttggggtgccaagc

SEQ ID NO: 64 - RNA, 16 nt, synthetic construct
  source 1..16: mol_type = other RNA
gctgacgaaagtcagc

SEQ ID NO: 65 - RNA, 16 nt, synthetic construct
  source 1..16: mol_type = other RNA
gctgaggagactcagc

SEQ ID NO: 66 - RNA, 16 nt, synthetic construct
  source 1..16: mol_type = other RNA
gctgtcggtggacagc

SEQ ID NO: 67 - RNA, 16 nt, synthetic construct
  source 1..16: mol_type = other RNA
gcgtacgaaagtacgc

SEQ ID NO: 68 - RNA, 16 nt, synthetic construct
  source 1..16: mol_type = other RNA
gcgtccgagaggacgc

SEQ ID NO: 69 - RNA, 16 nt, synthetic construct
  source 1..16: mol_type = other RNA
gcgttcggtggaacgc

SEQ ID NO: 70 - RNA, 16 nt, synthetic construct
  source 1..16: mol_type = other RNA
gcgggggaaaccccgc

SEQ ID NO: 71 - RNA, 16 nt, synthetic construct
  source 1..16: mol_type = other RNA
gcgggggagaccccgc

SEQ ID NO: 72 - RNA, 16 nt, synthetic construct
  source 1..16: mol_type = other RNA
gcggggggtgccccgc

SEQ ID NO: 73 - RNA, 18 nt, synthetic construct
  source 1..18: mol_type = other RNA
acttctcgaaagagaagt

SEQ ID NO: 74 - RNA, 18 nt, synthetic construct
  source 1..18: mol_type = other RNA
acttatggagacataagt

SEQ ID NO: 75 - RNA, 18 nt, synthetic construct
  source 1..18: mol_type = other RNA
acttgctggtgagcaagt

SEQ ID NO: 76 - RNA, 18 nt, synthetic construct
  source 1..18: mol_type = other RNA
actgcgcgaaagcgcagt

SEQ ID NO: 77 - RNA, 18 nt, synthetic construct
  source 1..18: mol_type = other RNA
actgcccgagagggcagt

SEQ ID NO: 78 - RNA, 18 nt, synthetic construct
  source 1..18: mol_type = other RNA
actgcgcggtggcgcagt

SEQ ID NO: 79 - RNA, 18 nt, synthetic construct
  source 1..18: mol_type = other RNA
acgtggggaaacccacgt

SEQ ID NO: 80 - RNA, 18 nt, synthetic construct
  source 1..18: mol_type = other RNA
acgtcgcgagagcgacgt

SEQ ID NO: 81 - RNA, 18 nt, synthetic construct
  source 1..18: mol_type = other RNA
acgtagcggtggctacgt

SEQ ID NO: 82 - RNA, 18 nt, synthetic construct
  source 1..18: mol_type = other RNA
acggagggaaacctccgt

SEQ ID NO: 83 - RNA, 18 nt, synthetic construct
  source 1..18: mol_type = other RNA
acggatggagacatccgt

SEQ ID NO: 84 - RNA, 18 nt, synthetic construct
  source 1..18: mol_type = other RNA
acggcgcggtggcgccgt

SEQ ID NO: 85 - RNA, 18 nt, synthetic construct
  source 1..18: mol_type = other RNA
gcttccagaaatggaagc

SEQ ID NO: 86 - RNA, 18 nt, synthetic construct
  source 1..18: mol_type = other RNA
gcttggggagacccaagc

SEQ ID NO: 87 - RNA, 18 nt, synthetic construct
  source 1..18: mol_type = other RNA
gcttcccggtggggaagc

SEQ ID NO: 88 - RNA, 18 nt, synthetic construct
  source 1..18: mol_type = other RNA
gctgggagaaatcccagc

SEQ ID NO: 89 - RNA, 18 nt, synthetic construct
  source 1..18: mol_type = other RNA
gctgggggagaccccagc

SEQ ID NO: 90 - RNA, 18 nt, synthetic construct
  source 1..18: mol_type = other RNA
gctggccggtgggccagc

SEQ ID NO: 91 - RNA, 18 nt, synthetic construct
  source 1..18: mol_type = other RNA
gcgttccgaaaggaacgc

SEQ ID NO: 92 - RNA, 18 nt, synthetic construct
  source 1..18: mol_type = other RNA
gcgtctggagacagacgc

SEQ ID NO: 93 - RNA, 18 nt, synthetic construct
  source 1..18: mol_type = other RNA
gcgttcgggtgcgaacgc

SEQ ID NO: 94 - RNA, 18 nt, synthetic construct
  source 1..18: mol_type = other RNA
gcggccggaaacggccgc

SEQ ID NO: 95 - RNA, 18 nt, synthetic construct
  source 1..18: mol_type = other RNA
gcggagggagacctccgc

SEQ ID NO: 96 - RNA, 18 nt, synthetic construct
  source 1..18: mol_type = other RNA
gcgggccggtgggcccgc

SEQ ID NO: 97 - RNA, 20 nt, synthetic construct
  source 1..20: mol_type = other RNA
acttccccgaaaggggaagt

SEQ ID NO: 98 - RNA, 20 nt, synthetic construct
  source 1..20: mol_type = other RNA
acttccccgagaggggaagt

SEQ ID NO: 99 - RNA, 20 nt, synthetic construct
  source 1..20: mol_type = other RNA
acttgccgggtgcggcaagt

SEQ ID NO: 100 - RNA, 20 nt, synthetic construct
  source 1..20: mol_type = other RNA
actgccacgaaagtggcagt

SEQ ID NO: 101 - RNA, 20 nt, synthetic construct
  source 1..20: mol_type = other RNA
actggacggagacgtccagt

SEQ ID NO: 102 - RNA, 20 nt, synthetic construct
  source 1..20: mol_type = other RNA
actgcagcggtggctgcagt

SEQ ID NO: 103 - RNA, 20 nt, synthetic construct
  source 1..20: mol_type = other RNA
acgtctgggaaaccagacgt

SEQ ID NO: 104 - RNA, 20 nt, synthetic construct
  source 1..20: mol_type = other RNA
acgtccgggagaccggacgt

SEQ ID NO: 105 - RNA, 20 nt, synthetic construct
  source 1..20: mol_type = other RNA
acgtgggaggtgtcccacgt

SEQ ID NO: 106 - RNA, 20 nt, synthetic construct
  source 1..20: mol_type = other RNA
acgggctggaaacagcccgt

SEQ ID NO: 107 - RNA, 20 nt, synthetic construct
  source 1..20: mol_type = other RNA
acgggggggagaccccccgt

SEQ ID NO: 108 - RNA, 20 nt, synthetic construct
  source 1..20: mol_type = other RNA
acggctcgggtgcgagccgt

SEQ ID NO: 109 - RNA, 20 nt, synthetic construct
  source 1..20: mol_type = other RNA
gcttccacgaaagtggaagc

SEQ ID NO: 110 - RNA, 20 nt, synthetic construct
  source 1..20: mol_type = other RNA
gcttactcgagagagtaagc

SEQ ID NO: 111 - RNA, 20 nt, synthetic construct
  source 1..20: mol_type = other RNA
gctttcccggtggggaaagc

SEQ ID NO: 112 - RNA, 20 nt, synthetic construct
  source 1..20: mol_type = other RNA
gctgacgcgaaagcgtcagc

SEQ ID NO: 113 - RNA, 20 nt, synthetic construct
  source 1..20: mol_type = other RNA
gctgacgggagaccgtcagc

SEQ ID NO: 114 - RNA, 20 nt, synthetic construct
  source 1..20: mol_type = other RNA
gctgatccggtgggatcagc

SEQ ID NO: 115 - RNA, 20 nt, synthetic construct
  source 1..20: mol_type = other RNA
gcgtgcacgaaagtgcacgc

SEQ ID NO: 116 - RNA, 20 nt, synthetic construct
  source 1..20: mol_type = other RNA
gcgtctgcgagagcagacgc

SEQ ID NO: 117 - RNA, 20 nt, synthetic construct
  source 1..20: mol_type = other RNA
gcgtgcggggtgccgcacgc

SEQ ID NO: 118 - RNA, 20 nt, synthetic construct
  source 1..20: mol_type = other RNA
gcggttccgaaaggaaccgc

SEQ ID NO: 119 - RNA, 20 nt, synthetic construct
  source 1..20: mol_type = other RNA
gcggccatgagaatggccgc

SEQ ID NO: 120 - RNA, 20 nt, synthetic construct
  source 1..20: mol_type = other RNA
gcggtggcggtggccaccgc

SEQ ID NO: 121 - RNA, 22 nt, synthetic construct
  source 1..22: mol_type = other RNA
acttaaagcgaaagctttaagt

SEQ ID NO: 122 - RNA, 22 nt, synthetic construct
  source 1..22: mol_type = other RNA
acttcgtccgagaggacgaagt

SEQ ID NO: 123 - RNA, 22 nt, synthetic construct
  source 1..22: mol_type = other RNA
acttagagcggtggctctaagt

SEQ ID NO: 124 - RNA, 22 nt, synthetic construct
  source 1..22: mol_type = other RNA
actgcgctcgaaagagcgcagt

SEQ ID NO: 125 - RNA, 22 nt, synthetic construct
  source 1..22: mol_type = other RNA
actgggttggagacaacccagt

SEQ ID NO: 126 - RNA, 22 nt, synthetic construct
  source 1..22: mol_type = other RNA
actgctgcgggtgcgcagcagt

SEQ ID NO: 127 - RNA, 22 nt, synthetic construct
  source 1..22: mol_type = other RNA
acgtctccggaaacggagacgt

SEQ ID NO: 128 - RNA, 22 nt, synthetic construct
  source 1..22: mol_type = other RNA
acgtgggcagagatgcccacgt

SEQ ID NO: 129 - RNA, 22 nt, synthetic construct
  source 1..22: mol_type = other RNA
acgtaggggggtgcccctacgt

SEQ ID NO: 130 - RNA, 22 nt, synthetic construct
  source 1..22: mol_type = other RNA
acggatcgcgaaagcgatccgt

SEQ ID NO: 131 - RNA, 22 nt, synthetic construct
  source 1..22: mol_type = other RNA
acggcacccgagagggtgccgt

SEQ ID NO: 132 - RNA, 22 nt, synthetic construct
  source 1..22: mol_type = other RNA
acggcgccgggtgcggcgccgt

SEQ ID NO: 133 - RNA, 22 nt, synthetic construct
  source 1..22: mol_type = other RNA
gcttcacgcgaaagcgtgaagc

SEQ ID NO: 134 - RNA, 22 nt, synthetic construct
  source 1..22: mol_type = other RNA
gcttacggggagacccgtaagc

SEQ ID NO: 135 - RNA, 22 nt, synthetic construct
  source 1..22: mol_type = other RNA
gcttgagggggtgccctcaagc

SEQ ID NO: 136 - RNA, 22 nt, synthetic construct
  source 1..22: mol_type = other RNA
gctgcgagcgaaagctcgcagc

SEQ ID NO: 137 - RNA, 22 nt, synthetic construct
  source 1..22: mol_type = other RNA
gctgcgtacgagagtacgcagc

SEQ ID NO: 138 - RNA, 22 nt, synthetic construct
  source 1..22: mol_type = other RNA
gctggcgtcggtggacgccagc

SEQ ID NO: 139 - RNA, 22 nt, synthetic construct
  source 1..22: mol_type = other RNA
gcgtgaggggaaaccctcacgc

SEQ ID NO: 140 - RNA, 22 nt, synthetic construct
  source 1..22: mol_type = other RNA
gcgtcttccgagaggaagacgc

SEQ ID NO: 141 - RNA, 22 nt, synthetic construct
  source 1..22: mol_type = other RNA
gcgtcccggggtgccgggacgc

SEQ ID NO: 142 - RNA, 22 nt, synthetic construct
  source 1..22: mol_type = other RNA
gcggatggggaaacccatccgc

SEQ ID NO: 143 - RNA, 22 nt, synthetic construct
  source 1..22: mol_type = other RNA
gcggaggccgagaggcctccgc

SEQ ID NO: 144 - RNA, 22 nt, synthetic construct
  source 1..22: mol_type = other RNA
gcgggcgtcggtggacgcccgc

SEQ ID NO: 145 - RNA, 24 nt, synthetic construct
  source 1..24: mol_type = other RNA
acttgccctcgaaagagggcaagt

SEQ ID NO: 146 - RNA, 24 nt, synthetic construct
  source 1..24: mol_type = other RNA
acttcaggacgagagtcctgaagt

SEQ ID NO: 147 - RNA, 24 nt, synthetic construct
  source 1..24: mol_type = other RNA
actttccgggggtgcccggaaagt

SEQ ID NO: 148 - RNA, 24 nt, synthetic construct
  source 1..24: mol_type = other RNA
actggaatgggaaaccattccagt

SEQ ID NO: 149 - RNA, 24 nt, synthetic construct
  source 1..24: mol_type = other RNA
actgtaccgggagaccggtacagt

SEQ ID NO: 150 - RNA, 24 nt, synthetic construct
  source 1..24: mol_type = other RNA
actgcctcctggtgaggaggcagt

SEQ ID NO: 151 - RNA, 24 nt, synthetic construct
  source 1..24: mol_type = other RNA
acgtgaggcagaaatgcctcacgt

SEQ ID NO: 152 - RNA, 24 nt, synthetic construct
  source 1..24: mol_type = other RNA
acgtctaggggagaccctagacgt

SEQ ID NO: 153 - RNA, 24 nt, synthetic construct
  source 1..24: mol_type = other RNA
acgttccgagggtgctcggaacgt

SEQ ID NO: 154 - RNA, 24 nt, synthetic construct
  source 1..24: mol_type = other RNA
acggcgcaacgaaagttgcgccgt

SEQ ID NO: 155 - RNA, 24 nt, synthetic construct
  source 1..24: mol_type = other RNA
acgggcgcgcgagagcgcgcccgt

SEQ ID NO: 156 - RNA, 24 nt, synthetic construct
  source 1..24: mol_type = other RNA
acggacgccaggtgtggcgtccgt

SEQ ID NO: 157 - RNA, 24 nt, synthetic construct
  source 1..24: mol_type = other RNA
gcttcgatccgaaaggatcgaagc

SEQ ID NO: 158 - RNA, 24 nt, synthetic construct
  source 1..24: mol_type = other RNA
gcttcgtctggagacagacgaagc

SEQ ID NO: 159 - RNA, 24 nt, synthetic construct
  source 1..24: mol_type = other RNA
gcttggcctcggtggaggccaagc

SEQ ID NO: 160 - RNA, 24 nt, synthetic construct
  source 1..24: mol_type = other RNA
gctgatacccgaaagggtatcagc

SEQ ID NO: 161 - RNA, 24 nt, synthetic construct
  source 1..24: mol_type = other RNA
gctgaggaaggagacttcctcagc

SEQ ID NO: 162 - RNA, 24 nt, synthetic construct
  source 1..24: mol_type = other RNA
gctgagagctggtgagctctcagc

SEQ ID NO: 163 - RNA, 24 nt, synthetic construct
  source 1..24: mol_type = other RNA
gcgtgcacgcgaaagcgtgcacgc

SEQ ID NO: 164 - RNA, 24 nt, synthetic construct
  source 1..24: mol_type = other RNA
gcgtaactcggagacgagttacgc

SEQ ID NO: 165 - RNA, 24 nt, synthetic construct
  source 1..24: mol_type = other RNA
gcgtgcacgcggtggcgtgcacgc

SEQ ID NO: 166 - RNA, 24 nt, synthetic construct
  source 1..24: mol_type = other RNA
gcgggtagaggaaactctacccgc

SEQ ID NO: 167 - RNA, 24 nt, synthetic construct
  source 1..24: mol_type = other RNA
gcggcggtcggagacgaccgccgc

SEQ ID NO: 168 - RNA, 24 nt, synthetic construct
  source 1..24: mol_type = other RNA
gcggcccgcaggtgtgcgggccgc

SEQ ID NO: 169 - RNA, 14 nt, synthetic construct
  source 1..14: mol_type = other RNA
agcttgaaaaagct

SEQ ID NO: 170 - RNA, 14 nt, synthetic construct
  source 1..14: mol_type = other RNA
agcttgagaaagct

SEQ ID NO: 171 - RNA, 14 nt, synthetic construct
  source 1..14: mol_type = other RNA
agcttggtgaagct

SEQ ID NO: 172 - RNA, 14 nt, synthetic construct
  source 1..14: mol_type = other RNA
acctggaaacaggt

SEQ ID NO: 173 - RNA, 14 nt, synthetic construct
  source 1..14: mol_type = other RNA
agctggagacagct

SEQ ID NO: 174 - RNA, 14 nt, synthetic construct
  source 1..14: mol_type = other RNA
agctgggtgcagct

SEQ ID NO: 175 - RNA, 14 nt, synthetic construct
  source 1..14: mol_type = other RNA
agcgtgaaaacgct

SEQ ID NO: 176 - RNA, 14 nt, synthetic construct
  source 1..14: mol_type = other RNA
atcgtgagaacgat

SEQ ID NO: 177 - RNA, 14 nt, synthetic construct
  source 1..14: mol_type = other RNA
agcgtggtgacgct

SEQ ID NO: 178 - RNA, 14 nt, synthetic construct
  source 1..14: mol_type = other RNA
accgggaaaccggt

SEQ ID NO: 179 - RNA, 14 nt, synthetic construct
  source 1..14: mol_type = other RNA
accgggagaccggt

SEQ ID NO: 180 - RNA, 14 nt, synthetic construct
  source 1..14: mol_type = other RNA
accggggtgccggt

SEQ ID NO: 181 - RNA, 14 nt, synthetic construct
  source 1..14: mol_type = other RNA
ggcttgaaaaagcc

SEQ ID NO: 182 - RNA, 14 nt, synthetic construct
  source 1..14: mol_type = other RNA
gccttgagaaaggc

SEQ ID NO: 183 - RNA, 14 nt, synthetic construct
  source 1..14: mol_type = other RNA
gccttggtgaaggc

SEQ ID NO: 184 - RNA, 14 nt, synthetic construct
  source 1..14: mol_type = other RNA
ggctggaaacagcc

SEQ ID NO: 185 - RNA, 14 nt, synthetic construct
  source 1..14: mol_type = other RNA
gcctggagacaggc

SEQ ID NO: 186 - RNA, 14 nt, synthetic construct
  source 1..14: mol_type = other RNA
ggctgggtgcagcc

SEQ ID NO: 187 - RNA, 14 nt, synthetic construct
  source 1..14: mol_type = other RNA
gccgtgaaaacggc

SEQ ID NO: 188 - RNA, 14 nt, synthetic construct
  source 1..14: mol_type = other RNA
ggcgtgagaacgcc

SEQ ID NO: 189 - RNA, 14 nt, synthetic construct
  source 1..14: mol_type = other RNA
gccgtggtgacggc

SEQ ID NO: 190 - RNA, 14 nt, synthetic construct
  source 1..14: mol_type = other RNA
ggcgggaaaccgcc

SEQ ID NO: 191 - RNA, 14 nt, synthetic construct
  source 1..14: mol_type = other RNA
gccgggagaccggc

SEQ ID NO: 192 - RNA, 14 nt, synthetic construct
  source 1..14: mol_type = other RNA
ggcggggtgccgcc

SEQ ID NO: 193 - RNA, 16 nt, synthetic construct
  source 1..16: mol_type = other RNA
agccttgaaaaaggct

SEQ ID NO: 194 - RNA, 16 nt, synthetic construct
  source 1..16: mol_type = other RNA
aggcttgagaaagcct

SEQ ID NO: 195 - RNA, 16 nt, synthetic construct
  source 1..16: mol_type = other RNA
acgcttggtgaagcgt

SEQ ID NO: 196 - RNA, 16 nt, synthetic construct
  source 1..16: mol_type = other RNA
agcctggaaacaggct

SEQ ID NO: 197 - RNA, 16 nt, synthetic construct
  source 1..16: mol_type = other RNA
aggctggagacagcct

SEQ ID NO: 198 - RNA, 16 nt, synthetic construct
  source 1..16: mol_type = other RNA
accctgggtgcagggt

SEQ ID NO: 199 - RNA, 16 nt, synthetic construct
  source 1..16: mol_type = other RNA
agccgtgaaaacggct

SEQ ID NO: 200 - RNA, 16 nt, synthetic construct
  source 1..16: mol_type = other RNA
aggcgtgagaacgcct

SEQ ID NO: 201 - RNA, 16 nt, synthetic construct
  source 1..16: mol_type = other RNA
atccgtggtgacggat

SEQ ID NO: 202 - RNA, 16 nt, synthetic construct
  source 1..16: mol_type = other RNA
atccgggaaaccggat

SEQ ID NO: 203 - RNA, 16 nt, synthetic construct
  source 1..16: mol_type = other RNA
acgcgggagaccgcgt

SEQ ID NO: 204 - RNA, 16 nt, synthetic construct
  source 1..16: mol_type = other RNA
acccggggtgccgggt

SEQ ID NO: 205 - RNA, 16 nt, synthetic construct
  source 1..16: mol_type = other RNA
gcgcttgaaaaagcgc

SEQ ID NO: 206 - RNA, 16 nt, synthetic construct
  source 1..16: mol_type = other RNA
gggcttgagaaagccc

SEQ ID NO: 207 - RNA, 16 nt, synthetic construct
  source 1..16: mol_type = other RNA
gaccttggtgaaggtc

SEQ ID NO: 208 - RNA, 16 nt, synthetic construct
  source 1..16: mol_type = other RNA
gccctggaaacagggc

SEQ ID NO: 209 - RNA, 16 nt, synthetic construct
  source 1..16: mol_type = other RNA
gccctggagacagggc

SEQ ID NO: 210 - RNA, 16 nt, synthetic construct
  source 1..16: mol_type = other RNA
gggctgggtgcagccc

SEQ ID NO: 211 - RNA, 16 nt, synthetic construct
  source 1..16: mol_type = other RNA
gcgcgtgaaaacgcgc

SEQ ID NO: 212 - RNA, 16 nt, synthetic construct
  source 1..16: mol_type = other RNA
ggccgtgagaacggcc

SEQ ID NO: 213 - RNA, 16 nt, synthetic construct
  source 1..16: mol_type = other RNA
gggcgtggtgacgccc

SEQ ID NO: 214 - RNA, 16 nt, synthetic construct
  source 1..16: mol_type = other RNA
ggccgggaaaccggcc

SEQ ID NO: 215 - RNA, 16 nt, synthetic construct
  source 1..16: mol_type = other RNA
gcccgggagaccgggc

SEQ ID NO: 216 - RNA, 16 nt, synthetic construct
  source 1..16: mol_type = other RNA
gctcggggtgccgagc

SEQ ID NO: 217 - RNA, 18 nt, synthetic construct
  source 1..18: mol_type = other RNA
atcccttgaaaaagggat

SEQ ID NO: 218 - RNA, 18 nt, synthetic construct
  source 1..18: mol_type = other RNA
acagcttgagaaagctgt

SEQ ID NO: 219 - RNA, 18 nt, synthetic construct
  source 1..18: mol_type = other RNA
acggcttggtgaagccgt

SEQ ID NO: 220 - RNA, 18 nt, synthetic construct
  source 1..18: mol_type = other RNA
aggtctggaaacagacct

SEQ ID NO: 221 - RNA, 18 nt, synthetic construct
  source 1..18: mol_type = other RNA
agggctggagacagccct

SEQ ID NO: 222 - RNA, 18 nt, synthetic construct
  source 1..18: mol_type = other RNA
aaggctgggtgcagcctt

SEQ ID NO: 223 - RNA, 18 nt, synthetic construct
  source 1..18: mol_type = other RNA
accccgtgaaaacggggt

SEQ ID NO: 224 - RNA, 18 nt, synthetic construct
  source 1..18: mol_type = other RNA
accgcgtgagaacgcggt

SEQ ID NO: 225 - RNA, 18 nt, synthetic construct
  source 1..18: mol_type = other RNA
agcgcgtggtgacgcgct

SEQ ID NO: 226 - RNA, 18 nt, synthetic construct
  source 1..18: mol_type = other RNA
atcccgggaaaccgggat

SEQ ID NO: 227 - RNA, 18 nt, synthetic construct
  source 1..18: mol_type = other RNA
agggcgggagaccgccct

SEQ ID NO: 228 - RNA, 18 nt, synthetic construct
  source 1..18: mol_type = other RNA
accccggggtgccggggt

SEQ ID NO: 229 - RNA, 18 nt, synthetic construct
  source 1..18: mol_type = other RNA
gagccttgaaaaaggctc

SEQ ID NO: 230 - RNA, 18 nt, synthetic construct
  source 1..18: mol_type = other RNA
gcgccttgagaaaggcgc

SEQ ID NO: 231 - RNA, 18 nt, synthetic construct
  source 1..18: mol_type = other RNA
gggacttggtgaagtccc

SEQ ID NO: 232 - RNA, 18 nt, synthetic construct
  source 1..18: mol_type = other RNA
gccgctggaaacagcggc

SEQ ID NO: 233 - RNA, 18 nt, synthetic construct
  source 1..18: mol_type = other RNA
gccgctggagacagcggc

SEQ ID NO: 234 - RNA, 18 nt, synthetic construct
  source 1..18: mol_type = other RNA
gccactgggtgcagtggc

SEQ ID NO: 235 - RNA, 18 nt, synthetic construct
  source 1..18: mol_type = other RNA
ggggcgtgaaaacgcccc

SEQ ID NO: 236 - RNA, 18 nt, synthetic construct
  source 1..18: mol_type = other RNA
gcaccgtgagaacggtgc

SEQ ID NO: 237 - RNA, 18 nt, synthetic construct
  source 1..18: mol_type = other RNA
ggggcgtggtgacgcccc

SEQ ID NO: 238 - RNA, 18 nt, synthetic construct
  source 1..18: mol_type = other RNA
gacccgggaaaccgggtc

SEQ ID NO: 239 - RNA, 18 nt, synthetic construct
  source 1..18: mol_type = other RNA
ggaccgggagaccggtcc

SEQ ID NO: 240 - RNA, 18 nt, synthetic construct
  source 1..18: mol_type = other RNA
gggccggggtgccggccc

SEQ ID NO: 241 - RNA, 20 nt, synthetic construct
  source 1..20: mol_type = other RNA
agaaccttgaaaaaggttct

SEQ ID NO: 242 - RNA, 20 nt, synthetic construct
  source 1..20: mol_type = other RNA
acagtcttgagaaagactgt

SEQ ID NO: 243 - RNA, 20 nt, synthetic construct
  source 1..20: mol_type = other RNA
aagcgcttggtgaagcgctt

SEQ ID NO: 244 - RNA, 20 nt, synthetic construct
  source 1..20: mol_type = other RNA
atcacctggaaacaggtgat

SEQ ID NO: 245 - RNA, 20 nt, synthetic construct
  source 1..20: mol_type = other RNA
agggcctggagacaggccct

SEQ ID NO: 246 - RNA, 20 nt, synthetic construct
  source 1..20: mol_type = other RNA
ataccctgggtgcagggtat

SEQ ID NO: 247 - RNA, 20 nt, synthetic construct
  source 1..20: mol_type = other RNA
aggctcgtgaaaacgagcct

SEQ ID NO: 248 - RNA, 20 nt, synthetic construct
  source 1..20: mol_type = other RNA
acctacgtgagaacgtaggt

SEQ ID NO: 249 - RNA, 20 nt, synthetic construct
  source 1..20: mol_type = other RNA
acccacgtggtgacgtgggt

SEQ ID NO: 250 - RNA, 20 nt, synthetic construct
  source 1..20: mol_type = other RNA
agaagcgggaaaccgcttct

SEQ ID NO: 251 - RNA, 20 nt, synthetic construct
  source 1..20: mol_type = other RNA
agggccgggagaccggccct

SEQ ID NO: 252 - RNA, 20 nt, synthetic construct
  source 1..20: mol_type = other RNA
acgcccggggtgccgggcgt

SEQ ID NO: 253 - RNA, 20 nt, synthetic construct
  source 1..20: mol_type = other RNA
gcctgcttgaaaaagcaggc

SEQ ID NO: 254 - RNA, 20 nt, synthetic construct
  source 1..20: mol_type = other RNA
ggatccttgagaaaggatcc

SEQ ID NO: 255 - RNA, 20 nt, synthetic construct
  source 1..20: mol_type = other RNA
gtcggcttggtgaagccgac

SEQ ID NO: 256 - RNA, 20 nt, synthetic construct
  source 1..20: mol_type = other RNA
gcggtctggaaacagaccgc

SEQ ID NO: 257 - RNA, 20 nt, synthetic construct
  source 1..20: mol_type = other RNA
gcgacctggagacaggtcgc

SEQ ID NO: 258 - RNA, 20 nt, synthetic construct
  source 1..20: mol_type = other RNA
gcgacctgggtgcaggtcgc

SEQ ID NO: 259 - RNA, 20 nt, synthetic construct
  source 1..20: mol_type = other RNA
ggggccgtgaaaacggcccc

SEQ ID NO: 260 - RNA, 20 nt, synthetic construct
  source 1..20: mol_type = other RNA
gtaggcgtgagaacgcctac

SEQ ID NO: 261 - RNA, 20 nt, synthetic construct
  source 1..20: mol_type = other RNA
gagcccgtggtgacgggctc

SEQ ID NO: 262 - RNA, 20 nt, synthetic construct
  source 1..20: mol_type = other RNA
gcccccgggaaaccgggggc

SEQ ID NO: 263 - RNA, 20 nt, synthetic construct
  source 1..20: mol_type = other RNA
ggcgacgggagaccgtcgcc

SEQ ID NO: 264 - RNA, 20 nt, synthetic construct
  source 1..20: mol_type = other RNA
gggcacggggtgccgtgccc

SEQ ID NO: 265 - RNA, 22 nt, synthetic construct
  source 1..22: mol_type = other RNA
accggccttgaaaaaggccggt

SEQ ID NO: 266 - RNA, 22 nt, synthetic construct
  source 1..22: mol_type = other RNA
atggggcttgagaaagccccat

SEQ ID NO: 267 - RNA, 22 nt, synthetic construct
  source 1..22: mol_type = other RNA
atcctacttggtgaagtaggat

SEQ ID NO: 268 - RNA, 22 nt, synthetic construct
  source 1..22: mol_type = other RNA
agcgggctggaaacagcccgct

SEQ ID NO: 269 - RNA, 22 nt, synthetic construct
  source 1..22: mol_type = other RNA
agcaccctggagacagggtgct

SEQ ID NO: 270 - DNA, 22 bp, synthetic construct
  source 1..22: mol_type = other DNA
agacctctgggtgcagaggtct

SEQ ID NO: 271 - RNA, 22 nt, synthetic construct
  source 1..22: mol_type = other RNA
acacgtcgtgaaaacgacgtgt

SEQ ID NO: 272 - RNA, 22 nt, synthetic construct
  source 1..22: mol_type = other RNA
acggggcgtgagaacgccccgt

SEQ ID NO: 273 - RNA, 22 nt, synthetic construct
  source 1..22: mol_type = other RNA
aggtggcgtggtgacgccacct

SEQ ID NO: 274 - RNA, 22 nt, synthetic construct
  source 1..22: mol_type = other RNA
atcccgcgggaaaccgcgggat

SEQ ID NO: 275 - RNA, 22 nt, synthetic construct
  source 1..22: mol_type = other RNA
agggtccgggagaccggaccct

SEQ ID NO: 276 - RNA, 22 nt, synthetic construct
  source 1..22: mol_type = other RNA
agtaggcggggtgccgcctact

SEQ ID NO: 277 - RNA, 22 nt, synthetic construct
  source 1..22: mol_type = other RNA
ggacgccttgaaaaaggcgtcc

SEQ ID NO: 278 - RNA, 22 nt, synthetic construct
  source 1..22: mol_type = other RNA
gcgacgcttgagaaagcgtcgc

SEQ ID NO: 279 - RNA, 22 nt, synthetic construct
  source 1..22: mol_type = other RNA
gtcggccttggtgaaggccgac

SEQ ID NO: 280 - RNA, 22 nt, synthetic construct
  source 1..22: mol_type = other RNA
gtcctcctggaaacaggaggac

SEQ ID NO: 281 - RNA, 22 nt, synthetic construct
  source 1..22: mol_type = other RNA
gtgcccctggagacaggggcac

SEQ ID NO: 282 - RNA, 22 nt, synthetic construct
  source 1..22: mol_type = other RNA
gtctgcctgggtgcaggcagac

SEQ ID NO: 283 - RNA, 22 nt, synthetic construct
  source 1..22: mol_type = other RNA
ggctaccgtgaaaacggtagcc

SEQ ID NO: 284 - RNA, 22 nt, synthetic construct
  source 1..22: mol_type = other RNA
gcggggcgtgagaacgccccgc

SEQ ID NO: 285 - RNA, 22 nt, synthetic construct
  source 1..22: mol_type = other RNA
gctcaccgtggtgacggtgagc

SEQ ID NO: 286 - RNA, 22 nt, synthetic construct
  source 1..22: mol_type = other RNA
gctagccgggaaaccggctagc

SEQ ID NO: 287 - RNA, 22 nt, synthetic construct
  source 1..22: mol_type = other RNA
ggcaggcgggagaccgcctgcc

SEQ ID NO: 288 - RNA, 22 nt, synthetic construct
  source 1..22: mol_type = other RNA
ggccagcggggtgccgctggcc

SEQ ID NO: 289 - RNA, 24 nt, synthetic construct
  source 1..24: mol_type = other RNA
accactgcttgaaaaagcagtggt

SEQ ID NO: 290 - RNA, 24 nt, synthetic construct
  source 1..24: mol_type = other RNA
actgtggcttgagaaagccacagt

SEQ ID NO: 291 - RNA, 24 nt, synthetic construct
  source 1..24: mol_type = other RNA
agcgccgcttggtgaagcggcgct

SEQ ID NO: 292 - RNA, 24 nt, synthetic construct
  source 1..24: mol_type = other RNA
agccaggctggaaacagcctggct

SEQ ID NO: 293 - RNA, 24 nt, synthetic construct
  source 1..24: mol_type = other RNA
agagcccctggagacaggggctct

SEQ ID NO: 294 - RNA, 24 nt, synthetic construct
  source 1..24: mol_type = other RNA
agcacccctgggtgcaggggtgct

SEQ ID NO: 295 - RNA, 24 nt, synthetic construct
  source 1..24: mol_type = other RNA
aacccggcgtgaaaacgccgggtt

SEQ ID NO: 296 - RNA, 24 nt, synthetic construct
  source 1..24: mol_type = other RNA
acgctcccgtgagaacgggagcgt

SEQ ID NO: 297 - RNA, 24 nt, synthetic construct
  source 1..24: mol_type = other RNA
acaaggccgtggtgacggccttgt

SEQ ID NO: 298 - RNA, 24 nt, synthetic construct
  source 1..24: mol_type = other RNA
acggccacgggaaaccgtggccgt

SEQ ID NO: 299 - RNA, 24 nt, synthetic construct
  source 1..24: mol_type = other RNA
atgcggacgggagaccgtccgcat

SEQ ID NO: 300 - RNA, 24 nt, synthetic construct
  source 1..24: mol_type = other RNA
agacagccggggtgccggctgtct

SEQ ID NO: 301 - RNA, 24 nt, synthetic construct
  source 1..24: mol_type = other RNA
gacagagcttgaaaaagctctgtc

SEQ ID NO: 302 - RNA, 24 nt, synthetic construct
  source 1..24: mol_type = other RNA
gtcgcagcttgagaaagctgcgac

SEQ ID NO: 303 - RNA, 24 nt, synthetic construct
  source 1..24: mol_type = other RNA
gtggccgcttggtgaagcggccac

SEQ ID NO: 304 - RNA, 24 nt, synthetic construct
  source 1..24: mol_type = other RNA
ggtcctcctggaaacaggaggacc

SEQ ID NO: 305 - RNA, 24 nt, synthetic construct
  source 1..24: mol_type = other RNA
gtccccgctggagacagcggggac

SEQ ID NO: 306 - RNA, 24 nt, synthetic construct
  source 1..24: mol_type = other RNA
ggtgggcctgggtgcaggcccacc

SEQ ID NO: 307 - RNA, 24 nt, synthetic construct
  source 1..24: mol_type = other RNA
gtggctccgtgaaaacggagccac

SEQ ID NO: 308 - RNA, 24 nt, synthetic construct
  source 1..24: mol_type = other RNA
gctccgacgtgagaacgtcggagc

SEQ ID NO: 309 - RNA, 24 nt, synthetic construct
  source 1..24: mol_type = other RNA
gtctccccgtggtgacggggagac

SEQ ID NO: 310 - RNA, 24 nt, synthetic construct
  source 1..24: mol_type = other RNA
gcgagcccgggaaaccgggctcgc

SEQ ID NO: 311 - RNA, 24 nt, synthetic construct
  source 1..24: mol_type = other RNA
ggacccccgggagaccgggggtcc

SEQ ID NO: 312 - RNA, 24 nt, synthetic construct
  source 1..24: mol_type = other RNA
gccctcccggggtgccgggagggc

SEQ ID NO: 313 - DNA, 186 bp, synthetic construct
  source 1..186: mol_type = other DNA
accggtcgtctccttttttatcgtttccgcttaacggcgaaacggtacgacagcgtgtgcggacaaggcaaggcttgaccgacaattggaagactcctatccgtcaacggagaccagatctggatgttccggagctccggtaccaaattgcatgaagccaaggctcacgatcggtgatggggatcc

SEQ ID NO: 314 - DNA, 51 bp, synthetic construct
  source 1..51: mol_type = other DNA
gttgcggatccaaaaaagcaccgactcggtgccactttcttaagttgataa

SEQ ID NO: 315 - DNA, 53 bp, synthetic construct
  source 1..53: mol_type = other DNA
cgttgcggatccaaaaaagcaccgactcggtgccactctttcgagttgataac

SEQ ID NO: 316 - DNA, 39 bp, synthetic construct
  source 1..39: mol_type = other DNA
ctgcactcgagtgcagcgaagacctgtttaagagctatg

SEQ ID NO: 317 - DNA, 40 bp, synthetic construct
  source 1..40: mol_type = other DNA
ctgcactcgagtgcagcgaagacctgttttagagctagaa

SEQ ID NO: 318 - DNA, 40 bp, synthetic construct
  source 1..40: mol_type = other DNA
gttgcggatccaaaaaagcaccgactcggtgccactttgc

SEQ ID NO: 319 - DNA, 30 bp, synthetic construct
  source 1..30: mol_type = other DNA
cgatttcttggctttatatatcttgtggaa

SEQ ID NO: 320 - DNA, 59 bp, synthetic construct
  source 1..59: mol_type = other DNA
agacctgtttaagagctatgctggaaacagcatagcaagtttaaataaggctagtccgt

SEQ ID NO: 321 - DNA, 59 bp, synthetic construct
  source 1..59: mol_type = other DNA
aaaaaagcaccgactcggtgccactttttcaagttgataacggactagccttatttaaa

SEQ ID NO: 322 - RNA, 54 nt, synthetic construct
  source 1..54: mol_type = other RNA
gacctgttttagagctagaaatagcaagttaaaataaggctagtccgttatcaa

SEQ ID NO: 323 - DNA, 54 bp, synthetic construct
  source 1..54: mol_type = other DNA
ccgactcggtgccactttgctgtttccagcaaagttgataacggactagcctta

SEQ ID NO: 324 - DNA, 45 bp, synthetic construct
  source 1..45: mol_type = other DNA
gggcacagataataacctgcaggagatctagagggcctatttccc

SEQ ID NO: 325 - DNA, 57 bp, synthetic construct
  source 1..57: mol_type = other DNA
cgcggatccaaaaaaggagacgaccggtcgtctccggtgtttcgtcctttccacaag

SEQ ID NO: 326 - DNA, 66 bp, synthetic construct
  source 1..66: mol_type = other DNA
caccgagctccggaacatccagatcgttttagagctagaaatagcaagttaaaataaggctagtcc

SEQ ID NO: 327 - DNA, 66 bp, synthetic construct
  source 1..66: mol_type = other DNA
taacggactagccttattttaacttgctatttctagctctaaaacgatctggatgttccggagctc

SEQ ID NO: 328 - DNA, 65 bp, synthetic construct
  source 1..65: mol_type = other DNA
gttatcaagagacgagcgtctctggcaccgagtcggtgcgagaccagattacctggatgttccgg

SEQ ID NO: 329 - DNA, 65 bp, synthetic construct
  source 1..65: mol_type = other DNA
aaaaccggaacatccaggtaatctggtctcgcaccgactcggtgccagagacgctcgtctcttga

SEQ ID NO: 330 - DNA, 20 bp, synthetic construct
  source 1..20: mol_type = other DNA
gaagcgtgatgacaaagagg

SEQ ID NO: 331 - DNA, 20 bp, synthetic construct
  source 1..20: mol_type = other DNA
ggagtgagggaaacggcccc

SEQ ID NO: 332 - DNA, 20 bp, synthetic construct
  source 1..20: mol_type = other DNA
atcgtttccgcttaacggcg

SEQ ID NO: 333 - RNA, 20 nt, synthetic construct
  source 1..20: mol_type = other RNA
aaacggtacgacagcgtgtg

SEQ ID NO: 334 - DNA, 20 bp, synthetic construct
  source 1..20: mol_type = other DNA
gtcacctccaatgactaggg

SEQ ID NO: 335 - DNA, 20 bp, synthetic construct
  source 1..20: mol_type = other DNA
gagtccgagcagaagaagaa

SEQ ID NO: 336 - DNA, 20 bp, synthetic construct
  source 1..20: mol_type = other DNA
ggaatcccttctgcagcacc

SEQ ID NO: 337 - DNA, 20 bp, synthetic construct
  source 1..20: mol_type = other DNA
ggcggctgcacaaccagtgg

SEQ ID NO: 338 - DNA, 20 bp, synthetic construct
  source 1..20: mol_type = other DNA
gcttgagaccgccagaagct

SEQ ID NO: 339 - DNA, 20 bp, synthetic construct
  source 1..20: mol_type = other DNA
cctggctaaactccacccat

SEQ ID NO: 340 - DNA, 20 bp, synthetic construct
  source 1..20: mol_type = other DNA
tcgagatgtgatgaaggaga

SEQ ID NO: 341 - DNA, 20 bp, synthetic construct
  source 1..20: mol_type = other DNA
ggccaggatggttcttaggt

SEQ ID NO: 342 - DNA, 20 bp, synthetic construct
  source 1..20: mol_type = other DNA
agctccggaacatccagatc

SEQ ID NO: 343 - DNA, 20 bp, synthetic construct
  source 1..20: mol_type = other DNA
cacccagaccatgaagatca

SEQ ID NO: 344 - DNA, 20 bp, synthetic construct
  source 1..20: mol_type = other DNA
cacccaaaccatgaagatca

SEQ ID NO: 345 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 346 - RNA, 87 nt, synthetic construct
  source 1..87: mol_type = other RNA
gtttaagagctatgctggaaacagcatagcaagtttaaataaggctagtccgttatcaacttgaaaaagtggcaccgagtcggtgct

SEQ ID NO: 347 - RNA, 87 nt, synthetic construct
  source 1..87: mol_type = other RNA
gtttaagagctatgctggaaacagcatagcaagtttaaataaggctagtccgttatcaactcgaaagagtggcaccgagtcggtgct

SEQ ID NO: 348 - RNA, 77 nt, synthetic construct
  source 1..77: mol_type = other RNA
gttttagagctagaaatagcaagttaaaataaggctagtccgttatcaactcgaaagagtggcaccgagtcggtgct

SEQ ID NO: 349 - RNA, 87 nt, synthetic construct
  source 1..87: mol_type = other RNA
gtttaagagctatgctggaaacagcatagcaagtttaaataaggctagtccgttatcaacttaagaaagtggcaccgagtcggtgct

SEQ ID NO: 350 - RNA, 77 nt, synthetic construct
  source 1..77: mol_type = other RNA
gttttagagctagaaatagcaagttaaaataaggctagtccgttatcaacttaagaaagtggcaccgagtcggtgct

SEQ ID NO: 351 - RNA, 87 nt, synthetic construct
  source 1..87: mol_type = other RNA
gttttagagctagaaatagcaagttaaaataaggctagtccgttatcaactttgctggaaacagcaaagtggcaccgagtcggtgct

SEQ ID NO: 352 - RNA, 77 nt, synthetic construct
  source 1..77: mol_type = other RNA
gttttagagctagaaatagcaagttaaaataaggctagtccgttatcagcggggtgccgcggcaccgagtcggtgct

SEQ ID NO: 353 - RNA, 83 nt, synthetic construct
  source 1..83: mol_type = other RNA
gttttagagctagaaatagcaagttaaaataaggctagtccgttatcagggccggggtgccggcccggcaccgagtcggtgct

SEQ ID NO: 354 - RNA, 89 nt, synthetic construct
  source 1..89: mol_type = other RNA
  misc_feature 49..73: note = up to 12 "n" residues can be deleted
gttttagagctagaaatagcaagttaaaataaggctagtccgttatcannnnnnnnnnnnnnnnnnnnnnnnggcaccgagtcggtgct

SEQ ID NO: 355 - RNA, 77 nt, synthetic construct
  source 1..77: mol_type = other RNA
gttttagagctagaaatagcaagttaaaataaggctagtccgttatcannnnnnnnnnnnggcaccgagtcggtgct

SEQ ID NO: 356 - RNA, 77 nt, synthetic construct; identical to SEQ ID NO: 355
  source 1..77: mol_type = other RNA

SEQ ID NO: 357 - RNA, 48 nt, synthetic construct
  source 1..48: mol_type = other RNA
gttttagagctagaaatagcaagttaaaataaggctagtccgttatca

SEQ ID NO: 358 - RNA, 16 nt, synthetic construct
  source 1..16: mol_type = other RNA
gcaccgagtcggtgct

SEQ ID NO: 359 - RNA, 18 nt, synthetic construct
  source 1..18: mol_type = other RNA
tatcaacttgaaaaagtg

SEQ ID NO: 360 - RNA, 12 nt, synthetic construct
  source 1..12: mol_type = other RNA
acttgaaaaagt

SEQ ID NO: 361 - DNA, 28 bp, synthetic construct
  source 1..28: mol_type = other DNA
gttgcggatccaaaaaagcaccgactcg

SEQ ID NO: 362 - RNA, 14 nt, synthetic construct
  source 1..14: mol_type = other RNA
aacttgaaaaagtg

SEQ ID NO: 363 - RNA, 14 nt, synthetic construct
  source 1..14: mol_type = other RNA
agcggggtgccgcg

SEQ ID NO: 364 - RNA, 20 nt, synthetic construct
  source 1..20: mol_type = other RNA
agggccggggtgccggcccg